SEQUENCE LISTING (28 sequences)

SEQ ID NO: 1 - protein, 1249 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQCANRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPG

SEQ ID NO: 2 - protein, 1249 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPG

SEQ ID NO: 3 - protein, 27 aa, synthetic construct
GYIPEAPRDGQAYVRKDGEWVLLSTFL

SEQ ID NO: 4 - protein, 1246 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGSASSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPG

SEQ ID NO: 5 - protein, 1236 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLG

SEQ ID NO: 6 - protein, 1247 aa, Severe acute respiratory syndrome coronavirus 2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPG

SEQ ID NO: 7 - protein, 1254 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 8 - protein, 1254 aa, synthetic construct
MFVFLVLLPLVSIQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLGVHKNNKSCMESEFGVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTHTNSHGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTLGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQGSGYIPEAPRDGQAYVRKDGEWVLLSTFLGRSLEVLFQGPGGGGGSEPEA

SEQ ID NO: 9 - protein, 1241 aa, synthetic construct
MFVFLVLLPLVSSQCVNLITRTQAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIGIRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRFITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPG

SEQ ID NO: 10 - protein, 1248 aa, synthetic construct
MFVFLVLLPLVSSQCVNITRTQAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIGIRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSKPCNGVEGFNCYFPLQSYGFQPTYGVGYQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRFITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 11 - protein, 1255 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 12 - protein, 1255 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDTSTEIYQVGSKPCNGVEGFNCYFPLQSYGFQPTNGVGYQPHRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 13 - protein, 1255 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFCEVFNATRFASVYAWNRKRISNCVAYYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPHRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 14 - protein, 1252 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVRRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDTSTEIYQVGSKPCNGVEGFNCYFPLQSYGFQPTNGVGYQPHRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 15 - protein, 1252 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVRRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFCEVFNATRFASVYAWNRKRISNCVAYYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLKESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 16 - protein, 1252 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITMFQTLLALHRSYLTPCYSSSCWTAGAAAYYVGYLQPRTFLLKYNENGTITDAVDCALDPLSETKCTLKSFTVEKGIYQTSNFRVQPTESIVRFPNITNLCPFCEVFNATRFASVYAWNRKRISNCVAYYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFCIDIADTTDAVRDPQTLEILDITPCSFCCVSVITPGTNTSNQVAVLYQGVNCTEVPVAIHADQLTPTWRVYSTGSNVFQTRAGCLIGAEHVNNSYECDIPIGAGICASYSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQIAYRFNGIGVTQNVLYENQKLIANQFNSAIGKIQDSLSSTPSALGKLQDVVNQNAQALNTLVKQLSSNFGAISSVLNDILSRLDPPEAEVQIDMFITGRLQSLQTYVTQQLIRAAEIRASANLAATKMSECVLGQSKRVDFCGKGYHLMSFPQSAPHGVVFLHVTYVPAQEKNFTTAPAICHDGKAHFPRECVFVSNCTHWFVTQRNFYEPQIITTDNTFVSGNCDVVIGIVNNTVYDPLQPELDSFKEELDKYFKNHTSPDVDLGDISGINASVVNIQKEIDRLNDVAKNLNESLIDLQELGKYEQGSGYIPEAPRDGQAYVRKDGEWVLLSTFLGRSLEVLFQGPGGGGSEPEA

SEQ ID NO: 17 - protein, 1251 aa, synthetic construct
MFVFLVLLPLVSSQCVNLITRTQAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIGREPEDLPQGFSALEPLVDLPIGINITMFQTLLALHRSYLTPCYSSSCWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVAYYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFCIDIADTTDAVRDPQTLEILDITPCSFCCVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRFITGRLQSLQTYVTQQLIRAAEIRASANLAATKMSECVLGQSKRVDFCGKGYHLMSFPQSAPHGVVFLHVTYVPAQEKNFTTAPAICHDGKAHFPRECVFVSNCTHWFVTQRNFYEPQIITTDNTFVSGNCDVVIGIVNNTVYDPLQPELDSFKEELDKYFKNHTSPDVDLGDISGINASVVNIQKEIDRLNDVAKNLNESLIDLQELGKYEQGSGYIPEAPRDGQAYVRKDGEWVLLSTFLGRSLEVLFQGPGGGGSEPEA

SEQ ID NO: 18 - protein, 1251 aa, synthetic construct
MFVFLVLLPLVSSQCVNLITRTQAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIGREPEDLPQGFSALEPLVDLPIGINITMFQTLLALHRSYLTPCYSSSCWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRFITGRLQSLQTYVTQQLIRAAEIRASANLAATKMSECVLGQSKRVDFCGKGYHLMSFPQSAPHGVVFLHVTYVPAQEKNFTTAPAICHDGKAHFPRECVFVSNCTHWFVTQRNFYEPQIITTDNTFVSGNCDVVIGIVNNTVYDPLQPELDSFKEELDKYFKNHTSPDVDLGDISGINASVVNIQKEIDRLNDVAKNLNESLIDLQELGKYEQGSGYIPEAPRDGQAYVRKDGEWVLLSTFLGRSLEVLFQGPGGGGSEPEA

SEQ ID NO: 19 - protein, 1255 aa, synthetic construct
MFVFLVLLPLVSSQCVNFITRTQLPSAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDVYNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGVENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGGGGSEPEA

SEQ ID NO: 20 - protein, 1271 aa, synthetic construct
MFVFLVLLPLVSSQCVNLITRTQAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIGREPEDLPQGFSALEPLVDLPIGINITMFQTLLALHRSYLTPCYSSSCWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVAYYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFCIDIADTTDAVRDPQTLEILDITPCSFCCVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRFITGRLQSLQTYVTQQLIRAAEIRASANLAATKMSECVLGQSKRVDFCGKGYHLMSFPQSAPHGVVFLHVTYVPAQEKNFTTAPAICHDGKAHFPRECVFVSNCTHWFVTQRNFYEPQIITTDNTFVSGNCDVVIGIVNNTVYDPLQPELDSFKEELDKYFKNHTSPDVDLGDISGINASVVNIQKEIDRLNDVAKNLNESLIDLQELGKYEQGSGYIPEAPRDGQAYVRKDGEWVLLSTFLGRSLEVLFQGPGGGGSGGGSEPEAGGGSEPEAGGGSEPEA

SEQ ID NO: 21 - DNA, 2092 bp, Severe acute respiratory syndrome coronavirus 2
  source 1..2092: mol_type = genomic DNA
atgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagcta

SEQ ID NO: 22 - DNA, 1757 bp, Severe acute respiratory syndrome coronavirus 2
  source 1..1757: mol_type = genomic DNA
gtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 23 - DNA, 1944 bp, synthetic construct
  source 1..1944: mol_type = other DNA
atgttcgtgttcctcgtgctgctgcccctggtgtcatcgcagtgcgtgaacctgaccacaagaactcagctgccgccagcttacaccaactccttcactcgcggagtgtactaccccgataaggtgttccgctcctccgtgctccactcgacccaagacctcttcctcccgttcttctcgaacgtgacctggttccacgccatccacgtgtccgggactaacggcaccaagcggttcgacaacccggtgcttcctttcaacgacggcgtctacttcgcgtccaccgagaaaagcaacattatccgcggctggatcttcggcactaccctggattcgaaaactcagtccctcctcatcgtgaacaacgctaccaatgtggtcatcaaagtctgcgagttccagttctgcaacgacccctttctcggggtgtactaccacaaaaacaacaagtcctggatggaatccgagttcagggtctactcaagcgcgaacaattgcaccttcgaatacgtgtcccagccgttcctcatggaccttgaagggaagcagggaaactttaagaatctcagggagttcgtgttcaagaacatcgacggctacttcaagatctactcgaagcacaccccgatcaaccttgtgcgggatctgccccaaggattttcagcgctcgaaccgctggttgatctccctatcgggatcaacatcacaagatttcagaccctgctcgcgctgcatcgctcctacctgacacccggagacagctcgtccggttggactgccggagctgcagcctactacgtgggctaccttcaacctcgcaccttcctgctgaagtataacgaaaacggaactattactgatgccgtggactgtgcgctggaccccctctccgaaaccaagtgtaccttgaagtccttcaccgtggagaagggaatctatcagacgagcaacttcagggtccagcctactgaatccatcgtgcggttccccaacatcactaatctctgtccgtttggcgaagtgttcaacgccaccagattcgcgtccgtgtacgcctggaaccgcaagaggatctccaactgcgtcgccgactacagcgtgctgtataactcagcgtccttctcgacctttaagtgctacggagtctcgcctaccaagttgaacgacctctgcttcaccaacgtgtacgcagactcgtttgtgatccggggagatgaagtccgacagatcgcccctggccaaaccgggaagattgccgactacaactacaagctgccagatgacttcaccggctgtgtgattgcgtggaactccaacaacctggactcgaaagtcggcgggaattacaactacctgtatcggctgttccggaagtccaacctgaagcctttcgaacgggacatctccactgaaatctaccaggccggttctaccccgtgcaacggtgtcgaggggttcaactgctactttcccctgcaatcatacgggttccagccgactaacggagtgggctaccagccctaccgggtggtggtgctgagcttcgagctcctgcatgctcctgccaccgtctgtggcccgaagaagtccaccaacctggtcaagaacaaatgtgtcaacttcaactttaacggcctgaccggaaccggagtgttgacagaaagcaacaagaagttccttccctttcaacaattcggtcgcgacattgccgacaccaccgatgcagtgcgggatccccaaaccctggaaattctggatatcaccccttgctccttcggtggagtcagcgtgatcactcctgggaccaatacctcgaaccaggtcgccgtactgtatcaggacgtgaattgcaccgaagtgccggtggccatccacgccgatcagcttacccccacttggagagtgtactcgaccggaagcaacgtgttccagactagagccggc

SEQ ID NO: 24 - DNA, 1806 bp, synthetic construct
  source 1..1806: mol_type = other DNA
tgcttgatcggcgctgaacacgtgaacaatagttacgagtgcgacatccccatcggtgccggaatatgcgcatcgtaccagacccagaccaattcccctggatctgcctcatccgtggcgtcccagtcgatcatcgcctacactatgtcactgggagccgaaaacagcgtggcatactccaacaattccatcgccatcccgactaacttcaccatttccgtgactaccgagattctgccagtgtctatgactaagacctccgtggactgcaccatgtatatctgcggagattccaccgaatgcagcaacctactgctgcaatacggttccttctgcacgcaactcaaccgggcactgaccggaattgccgtggagcaggataagaacacccaggaggtctttgcgcaagtcaagcagatctacaagactccgccgattaaggacttcggaggtttcaacttctcccaaatcctgcccgatccctccaaaccttcgaagcgctccccaattgaggatctgctgttcaacaaggtcaccctggctgatgccggcttcatcaagcaatacggcgattgcttgggggacattgcagctcgggacctcatttgcgcccaaaagttcaacggtctgaccgtcctgcccccattgttaaccgatgagatgatcgcccagtatactagcgcactgcttgccggcaccatcactagcgggtggaccttcggagcaggaccggctctccaaatcccgttcccgatgcaaatggcgtacaggttcaacggcatcggcgtgactcagaacgtgctgtacgagaaccagaagctgatcgcaaaccagttcaattcagccattggaaagattcaggacagcctttcctcaacgccttcagccctgggaaagctgcaggacgtcgtgaaccagaacgcccaggctctgaacaccctcgtgaagcagctgtccagcaactttggcgccattagctccgtgctgaacgatatcctgtcacggctggacccaccagaggccgaggtccagattgaccggctgattactggacgcctgcagagcctgcagacctacgtgactcagcagctgattagagccgccgagattagagccagcgccaatctggccgccacgaagatgtccgaatgcgtgttggggcagtcgaagcgcgtggacttctgtggaaagggataccacttgatgtctttcccacaatccgccccacatggcgtggtgttcctgcacgtgacctacgtgccggcgcaggaaaagaactttactactgcgccggcgatatgccacgacggaaaggcccacttccctcgggaaggagtgttcgtgagcaacggcactcactggttcgtgacccagcgcaacttctacgagccccagatcatcaccaccgacaacaccttcgtgtccggaaactgcgacgtggtcattggtattgtgaacaacaccgtgtacgaccccctccaacccgagctggactccttcaaggaagaattggacaagtacttcaagaaccacacgtcaccggacgtggatctcggagacatctccggcattaacgcctcggtggtcaacattcagaaggagatcgaccggctgaatgaggtcgcaaagaacctgaacgagagcctgatcgacctccaagaacttgggaaatacgagcagggctccggttacatcccggaagctcctcgcgacggacaggcctacgtgcggaaggacggagaatgggtgctcctgtccactttcttgggacgcagcctcgaagtgctgttccaaggacctggctaa

SEQ ID NO: 25 - DNA, 1939 bp, synthetic construct
  source 1..1939: mol_type = other DNA
atgttcgtgttcctcgtgctgttacccctcgtgtcgattcagtgcgtgaatctcaccacccgcacacagcttccgcctgcgtacactaacagcttcacccggggagtgtattaccctgacaaagtgttcaggagctccgtcctgcactcaacgcaggacctgtttctgccgttcttctccaacgtgacctggttccacgtgatctccggcaccaacggaactaagcggttcgacaatcccgtgttgcccttcaacgatggagtgtacttcgcctcgattgaaaagtcgaatatcatccgaggatggattttcggaaccaccctggacagcaagacccagtccctccttatcgtcaacaacgcgactaatgtcgtgatcaaagtctgcgagttccagttctgcaacgacccttttctgggggtgcacaagaacaacaagtcgtgcatggagtccgaatttggcgtgtactcctccgccaacaactgtactttcgaatacgtgtcccagcctttcctgatggacctcgagggaaagcagggaaacttcaagaatctccgggagtttgtgttcaaaaacatcgacggttacttcaagatctactccaagcacacccccattaacctcgtgcgcgatctgccgcaaggcttctcagccctggaacccttggtggacctccccattggcattaacatcacacggttccagactctgcttgcgcttcaccgctcctacttgaccccgggaggatcgtcatcggggtggactgctggagccgctgcgtactacgtcggctacctccagccccggacttttctgctcaagtataacgagaacggcaccatcactgacgccgtggattgtgccctggacccactgtccgaaactaagtgcactctgaagtccttcaccgtggagaaaggaatctaccagacctccaacttccgggtgcagcctaccgagtcaatcgtgcggttccctaacatcaccaacctgtgtccgttcggggaggtctttaacgccactcgcttcgcctccgtgtacgcgtggaatagaaagcggattagcaactgcgtggccgactactcagtgctttacaactccgcctccttctccaccttcaagtgctacggagtgtccccgactaagctgaacgacctctgtttcaccaacgtctacgccgactccttcgtgatccggggcgatgaagtccgccagatcgcccctggccaaaccgggaagatcgccgactacaactacaagctgccggacgactttacgggatgcgtgatcgcatggaactcaaacaacctggactcaaaggtcggagggaactataactaccggtataggctgttccgcaagagcaacctcaagcccttcgagcgcgacataagcacggaaatctaccaagccggatccaccccctgtaacggcgtgaagggctttaactgctacttcccgctgcaatcctacggtttccagccgacctacggcgtgggataccagccgtatagggtggtggtgctctccttcgaactgctgcatgcaccggccaccgtgtgtggccctaagaagagtaccaacctcgtgaagaacaaatgcgtgaacttcaactttaacggactgaccggaaccggcgtgctgaccgagtcgaacaagaagttcttgccattccaacaattcggtagagacatcgatgacacgaccgacgccgtcagagatccgcagactctggagatcctcgacatcaccccgtgctccttcggcggagtgtcggtcattacccccggaaccaacacgtccaaccaagtggctgtgctgtaccagggcgtgaattgcaccgaggtgcccgtagccattcacgccgatcagctgaccccaacctggagggtttacagcaccggttcgaatgtgttccaaactcgcgcgggatgcctga

SEQ ID NO: 26 - DNA, 1799 bp, synthetic construct
  source 1..1799: mol_type = other DNA
ttggcgccgagcacgtgaacaactcctacgaatgcgacattcctatcggtgccggaatttgcgcctcctaccaaacccacaccaactcgcatggttccgcaagctcggtggcctcccaaagcatcatcgcctacactatgtccctgggagccgagaactcggtggcgtactccaacaactcaatcgccatccccactaacttcaccattagcgtgaccaccgaaattttgccggtgtcgatgaccaagacatccgtcgactgcaccatgtacatctgcggcgatagcactgagtgctcgaaccttctgctgcaatacggctccttctgtacccaactgaaccgggcgctaaccggaattgccgtcgagcaggacaaaaacacccaggaagtgttcgcccaagtcaagcagatctacaaaactccgcctattaaggacttcgggggattcaactttagccagattctgcccgatccctctaagccgtctaagaggtcccccattgaggatctgctgttcaacaaggtcaccctggccgatgctgggttcatcaagcagtacggagactgcctgggagacatcgcagctcgcgacctgatctgcgcccaaaagttcaacggactcaccgtcctgcccccactgctgactgacgaaatgatcgctcagtacaccagcgctctgctggctggcactattacctccgggtggacactgggagctggtcctgcacttcaaatccccttccctatgcaaatggcgtatcggttcaacggcatcggtgtcactcagaacgtcctctacgaaaaccagaagctgatcgcgaatcagttcaactccgccatcggaaagatccaggactcgctcagctccactccgtcggctctggggaagctgcagaatgtcgtgaaccagaatgcacaggccttgaacactctcgtgaagcagctcagctcgaacttcggcgcaatttcctccgtgcttaatgatatcttgtcccggcttgatccgcctgaggcagaggtgcagatcgataggctgatcaccggacggctccagagcctccagacctacgtgactcagcagctgattcgcgccgcggaaatcagagcctcagccaacctggccgccactaagatgagcgaatgcgtcctgggacagtccaaaagagtggacttctgcggaaagggctaccatctcatgtcctttccacaatccgcgccgcatggagtggtgttcctgcacgtgacctacgtgccggcccaggaaaagaacttcaccactgcgcctgccatctgtcatgatgggaaggcccacttcccgagagagggggtgttcgtgtcgaatggcacccactggttcgtcacccaacggaatttctacgaaccgcagatcataaccaccgacaacaccttcgtgtccgggaactgtgacgtggtcattggcattgtgaacaacactgtgtacgaccccctgcaaccagagctggactcattcaaggaggagctggataagtatttcaagaaccacacctcgccggacgtggatctcggagacatcagcggcatcaacgcctctttcgtcaacattcagaaggaaatcgaccggctcaacgaagtggccaagaacctgaacgaatccctgattgatctgcaagagctgggcaaatacgaacagggttccggctacatccctgaagcaccgcgcgatggacaggcctacgtgcgcaaggatggcgaatgggtgctgttgagcacgtttctggggagaagcttggaggtgctgtttcaagggcccggttaa

SEQ ID NO: 27 - protein, 4 aa, Severe acute respiratory syndrome coronavirus 2
RRAR

SEQ ID NO: 28 - protein, 4 aa, synthetic construct
GSAS

(19) United States
(12) Patent Application Publication
(10) Pub. No.: US 2025/0368689 A1
(43) Pub. Date: Dec. 4, 2025

(54) CROSS-REACTIVE CORONAVIRUS SPIKE PROTEIN AND METHODS OF USE THEREOF

(71) Applicants: EXCELLGENE SA, Monthey (CH); The University of Sydney, The University of Sydney NSW (AU); Centenary Institute of Cancer Medicine and Cell Biology, Camperdown, NSW (AU)

(72) Inventors: Paco PINO, Troistorrents (CH); Florian M. WURM, Montreux (CH); Joeri KINT, Lausanne (CH); Divor KISELJAK, Renens (CH); James TRICCAS, Ashbury (AU); Claudio COUNOUPAS, Erskineville (AU); Maria J. WURM, Montreux (CH)

(21) Appl. No.: 18/863,271

(22) PCT Filed: May 5, 2023
(86) PCT No.: PCT/IB2023/054719

§ 371 Date: Nov. 5, 2024

Related U.S. Application Data
(60) Provisional application No. 63/339,275, filed on May 6, 2022.

(51) Int. Cl. C07K 14/005; A61K 39/215; A61P 37/04

(52) U.S. Cl. CPC C07K 14/005; A61K 39/215; A61P 37/04; C12N 2770/20022; C12N 2770/20034

(57) ABSTRACT

Embodiments provided here include engineered viral proteins, such as but not limited to coronavirus spike proteins, e.g., SARS-CoV-2 spike proteins. These engineered viral proteins comprise modifications or mutations that facilitate secretion and efficient production. Exemplary engineered coronavirus spike proteins of the disclosure can combine mutations in regions of the spike proteins observed in various viruses of concern that have circulated in humans in one singular protein sequence. Additional embodiments provide nucleic acid molecules encoding the coronavirus spike proteins of the disclosure, pharmaceutical compositions and host cells comprising the proteins and/or nucleic acid molecules described here, methods and uses thereof for the prophylactic treatment of infection or disease associated with a coronavirus infection, and methods of producing such coronavirus spike proteins, as well as provide neutralizing antibody titers representing SARS-CoV-2 variants of concern, and high throughput, large scale bioreactor operation methods for production of human vaccines based on purified Spike proteins.

### CROSS-REFERENCE TO RELATED APPLICATIONS

This application is a 35 U.S.C. § 371 national stage application of International Patent Application No. PCT/IB2023/054719, filed May 5, 2023, which claims the benefit of U.S. Provisional Patent Application No. 63/339,275, filed May 6, 2022 and entitled “Cross-Reactive Coronavirus Spike Protein and Methods of Use Thereof,” all of which are hereby incorporated by reference in their entirety.

### SEQUENCE LISTING

The instant application contains a Sequence Listing which has been submitted electronically in XML format and is hereby incorporated by reference in its entirety. Said XML copy, created on Jul. 31, 2025, is named 178225-010202_US_SL.xml and is 60,472 bytes in size.

### FIELD OF DISCLOSURE

The present disclosure is generally related to the fields of virology, immunology, and cell biology. For example, the disclosure relates generally to engineered coronavirus spike proteins as well as variants thereof, vectors, and host cells containing such engineered coronavirus spike proteins, and methods of making and using such coronavirus spike proteins in the treatment and prevention of coronavirus infections, coronavirus disease 19 (COVID-19) or COVID-19-associated diseases, disorders, and conditions.

### BACKGROUND

Severe acute respiratory syndrome coronavirus 2 (SARS-CoV-2) is the virus that causes coronavirus disease 2019 (COVID-19). SARS-CoV-2 is an enveloped, single-stranded positive-sense RNA virus of the Coronaviridae family and particularly the genus of Betacoronaviruses. The genome of the Coronaviridae encodes at least the following the structural proteins: spike (S), envelope (E), membrane (M), and nucleocapsid protein (N). The spike protein is a transmembrane, homotrimeric, Class I fusion glycoprotein that enables the virus to penetrate host cells and cause infection by viral attachment, fusion, and entry of the virus into host cells. The full-length coronavirus spike protein generally comprises two portions, the S1 portion located at the N-terminal end and an S2 portion located at the C-terminal end of the protein. FIG. 1 provides an illustration showing the S1 portion comprising an N-Terminal Domain (NTD) and a Receptor-Binding Domain (RBD) and the S2 portion comprising a Fusion Peptide (FP) sequence, a Heptad Repeat 1 (HR1), a Heptad Repeat 2 (HR2), Transmembrane Anchor (TA)/Transmembrane Domain (TD), and Intracellular Tail (IT).

During SARS-CoV-2 infection, the coronavirus spike protein receptor-binding domain (RBD) binds to a host angiotensin-converting enzyme-2 (ACE-2) receptor in order to enter cells of the host. Cellular proteases target viral proteins such as coronavirus spike proteins, for cleavage. This, in turn induces a conformational change in the Spike protein that allows for membrane fusion and entry of the virus into the host cell. Since the receptors are genetically and structurally conserved among mammalian species, multiple animal coronaviruses are able to bind to the human ACE-2 receptor. The spike protein of SARS-CoV-2 is rapidly evolving, as demonstrated by the emergence of numerous variants of concern. The rapid evolution of the spike protein has motivated the development of vaccines with broad protection against existing as well as new, emerging variants. Moreover, in view of the rapid evolution and genetic recombination, even fully vaccinated people or people who have previously been infected can be reinfected and suffer from disease potentially spreading the virus further. There is therefore a need for protection against the ever evolving and mutating coronavirus, e.g., SARS-CoV-2 virus, and thus the need for generation of vaccines that protect against severe disease not only against one particular SARS-CoV-2 variant, but more than one.

### SUMMARY

As described here, the present disclosure and embodiments thereof feature an engineered coronavirus spike protein derived from sequences of betacoronaviruses, compositions, expression vectors, host cells, and efficient methods for manufacturing the coronavirus spike protein or encoding the coronavirus spike protein, methods for producing the engineered coronavirus spike protein, and methods for prophylactically treating a coronavirus infection, e.g., COVID-19 or coronavirus infection-associated diseases, disorders, and conditions, in a subject in need thereof with compositions comprising the engineered coronavirus spike protein described here or nucleic acid molecule encoding the same. As described in the various embodiments here, a desired vaccine or composition to elicit an immune response to protect against severe disease not only against one particular SARS-CoV-2 variant, but more than one can be composed of, for example, one trimeric spike molecule, exposing antigenic sites for several known past and potentially future virus variants. Other embodiments demonstrate that the recombinant engineered coronavirus spike proteins described here provide high levels of neutralizing antibodies against different virus variants of concern and are capable of inducing high-level neutralizing antibody levels in booster injections of subjects that were previously vaccinated.

Some aspects of the disclosure provide, a protein, comprising:
an engineered coronavirus spike protein comprising an amino acid sequence of at least 90% (e.g., 91%, 92%, 93%, 94%, 95%, 96%, 97%, 98%, 99%, 100%) identity to:

(SEQ ID NO: 1)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QCANRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG,

wherein the engineered coronavirus spike protein comprises at least one (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) mutation relative to the sequence, wherein the mutation is selected from the group consisting of: T19I; deletion of L24; deletion of P25-P26; deletion of V143; deletion of Y144-Y145; Y145N; deletion of E156; deletion of F157; R158G; deletion of N211; L212I; V213G; insertion of R214; R237M; G252C; D253Y; G257C; G339D; D364Y; V367F; S371L; S373P; S375F; T376A; D405N; R408S; N440K; G446S; I468T; A475V; S477N; E484A; Q493R; G496S; Q498R; Y505H; Y508H; T547K; G566C; R567I; A570D; G593C; G594C; deletion of: Q675, T676, Q677, T678, and N679; N679K; N764K; D796Y; N856K; M902I; Q954H; N969K; L981F; R995M; L996F; G1093C; G1099C; E1188D; and any combinations thereof.

Other aspects provide a protein, comprising:
an engineered coronavirus spike protein comprising an amino acid sequence of at least 90% (e.g., 91%, 92%, 93%, 94%, 95%, 96%, 97%, 98%, 99%, 100%) identity to:

(SEQ ID NO: 1)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QCANRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG,

wherein the engineered coronavirus spike protein comprises at least one mutation (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) relative to the sequence, wherein the mutation is selected from the group consisting of: 0-1 mutation of a signal peptide sequence (1-13 aa); 3-16 mutations of an N-terminal domain (13-305 aa); 2-19 mutations of a receptor binding domain (319-541aa); 2-10 mutations of a receptor binding motif (437-508 aa); 0-1 mutation of a fusion peptide sequence (788-806 aa); 0-3 mutations of a heptad repeat 1 (912-984 aa); 0-1 mutation of a heptad repeat 2 (1163-1213 aa); and any combinations thereof.

Further aspects provide a protein, comprising:
an engineered coronavirus spike protein comprising an amino acid sequence of at least 90% (e.g., 91%, 92%, 93%, 94%, 95%, 96/o, 97%, 98%, 99%, 100%) identity to:

(SEQ ID NO: 1)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QCANRFNGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG,

wherein the engineered coronavirus spike protein comprises a plurality of mutations (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) relative to the sequence, wherein the mutation is selected from the group consisting of:
S13I; A67V; deletion of H69 and V70; T95I; deletion of Y144 and Y145; W152C; R158G; D253G; L452R; E484K; N501Y; A570D; D614G; Q677H; P681H; F888L; D950N; V1176F; or
T19I; deletion of L24, P25, and P26; A67V; deletion of H69 and V70; T95I; G142D; deletion of V143; deletion of: Y144 and Y145; deletion of N211; L212I; V213G; insertion of R214; L452R; T478K; T547K; D614G; H655Y; N679K; P681H; N764K; D796Y; N856K; Q954H; N969K; L981F; or
T19I; deletion of L24, P25, and P26; A67V; deletion of H69 and V70; T95I; G142D; deletion of V143; deletion of: Y144 and Y145; deletion of N211; L212I; V213G; insertion of R214; K417N; E484K; N501Y; T547K; D614G; H655Y; N679K; P681H; N764K; D796Y; N856K; Q954H; N969K; L981F; or
T19R; deletion of E156; deletion of F157; R158G; G339D; S371L; S373P; S375F; K417N; N440K; G446S; S477N; T478K; E484A; Q493R; G496S; Q498R; N501Y; Y505H; D614G; P681R; D950N; or
T19R; deletion of E156; deletion of F157; R158G; V367F; L452R; I468T; A475V; T478K; Y508H; D614G; P681R; D950N; or
T19R; deletion of E156; deletion of F157; R158G; G339D; D364Y; L452R; T478K; Y508H; D614G; P681R; D950N; or
A67V; deletion of H69 and V70; T95I; G142D; deletion of V143; deletion of: Y144 and Y145; deletion of N211; L212I; insertion of R214; G339D; V367F; L452R; I468T; A475V; T478K; Y508H; T547K; D614G; H655Y; N679K; P681H; N764K; D796Y; N856K; Q954H; N969K; L981F; or
A67V; deletion of H69 and V70; T95I; G142D; deletion of V143; deletion of: Y144 and Y145; deletion of N211; L212I; insertion of R214; G339C; D364Y; L452R; T478K; T547K; D614G; H655Y; N679K; P681H; N764K; D796Y; N856K; Q954H; N969K; L981F; or
R237M; G252C; D253Y; G257C; G339C; D364Y; G566C; R567I; G593C; G594C; deletion of: Q675, T676, Q677, T678, N679; M902I; R995M; L996F; G1093C; G1099C; E1188D; or
T19I; deletion of L24; deletion of: P25 and P26; A67V; deletion of: H69 and V70; T95I; G142D; deletion of V143; deletion of: Y144 and Y145; deletion of N211; L212I; V213G; insertion of R214; R237M; G252C; D253Y; G257C; G339D; D364Y; S371F; S373P; S375F; T376A; D405N; R408S; K417N; N440K; G446S; S477N; T478K; E484A; Q493R; G4% S; Q498R; Y505H; T547K; G566C; R567I; G593C; G594C; D614G; H655Y; N679K; P681H; N764K; D796Y; N856K; Q954H; N969K; L981F; L996F; G1093C; G1099C; E1188D; or
T19I; deletion of L24; deletion of: P25 and P26; A67V; deletion of: H69 and V70; T95I; G142D; deletion of V143; deletion of: Y144 and Y145; deletion of N211; L212I; V213G; insertion of R214; R237M; G252C; D253Y; G257C; L452R; T478K; E484A; Q493R; G4% S; Q498R; N501Y; Y505H; T547K; D614G; H655Y; N679K; P681H; N764K; D796Y; N856K; Q954H; N969K; L981F; L996F; G1093C; G1099C; E1188D; or
L18F; T19I; P26S; A67V; deletion of: H69 and V70; T95I; G142D; Y145N; D253G; G339D; S371L; S373P; S375F; K417N; N440K; G446S; S477N; T478K; E484A; Q493R; G496S; Q498R; N501Y; Y505H; D614G; H655Y; N679K; P681H; A701V; N764K; D796Y; D950N; Q954H; N969K.

Further aspects provide a protein, comprising: an engineered coronavirus spike protein comprising an amino acid sequence of at least 90% identity to:

(SEQ ID NO: 2)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QMAYRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG
(FIG. 3E),

where the protein comprises more than one engineered coronavirus spike protein, where each engineered coronavirus spike protein comprises a virus variant-spike monomer capable of forming a homo-trimeric structure or a hetero-trimeric structure.

In some aspects, provided here is a nucleic acid molecule comprising a nucleotide sequence encoding an amino acid sequence of a protein comprising an engineered coronavirus spike protein described here.

Additional aspects provide a pharmaceutical composition comprising: a protein comprising an engineered coronavirus spike protein or a nucleic acid molecule encoding the engineered coronavirus spike protein, where the pharmaceutical composition comprises a pharmaceutically acceptable vehicle.

Further aspects provide a method, comprising:
prophylactically treating a coronavirus infection or disease associated with a coronavirus infection in a subject, wherein the treating comprises,
administering to the subject an effective amount of the protein comprising an engineered coronavirus spike protein, a nucleic acid molecule encoding the engineered coronavirus spike protein, or a pharmaceutical composition comprising a vehicle (e.g., carrier, excipient, diluent, adjuvant) and the engineered coronavirus spike protein or the nucleic acid molecule encoding the engineered coronavirus spike protein.

In some aspects, provided here is a use of a product, including the protein comprising an engineered coronavirus spike protein, a nucleic acid molecule encoding the engineered coronavirus spike protein, or a pharmaceutical composition comprising a vehicle (e.g., carrier, excipient, diluent, adjuvant) and the engineered coronavirus spike protein or the nucleic acid molecule encoding the engineered coronavirus spike protein, for prophylactically treating a coronavirus infection or disease associated with a coronavirus infection.

Additional aspects provide a method for producing a recombinant engineered coronavirus spike protein, comprising:
a. introducing into a host cell, an expression vector comprising a nucleic acid molecule encoding the engineered coronavirus spike protein of FIG. 2A-FIG. 2F and FIG. 3A-FIG. 3R;
b. culturing the host cell under conditions which allow for expression of the recombinant engineered coronavirus spike protein; and
c. isolating the recombinant engineered coronavirus spike protein from the cultured host cell, thereby producing the recombinant engineered coronavirus spike protein.

Further aspects provide a method for producing a recombinant engineered coronavirus spike protein, comprising:
a. introducing into a eukaryotic host cell, a first nucleic acid sequence encoding an engineered coronavirus spike protein mediated by the provisioning of an additional nucleic acid sequence encoding a transposase;
b. culturing the eukaryotic host cell under conditions which allow expression of the first nucleic acid sequence encoding an engineered coronavirus spike protein;
c. selecting the eukaryotic host cell with the nucleic acid molecule expressing an engineered coronavirus spike protein, wherein the selected cells are clonally-derived cells expressing recombinant engineered coronavirus spike protein; and
d. isolating the recombinant engineered coronavirus spike protein from the clonally-derived cells, thereby producing the recombinant engineered coronavirus spike protein.

In some aspects, provided here is an expression vector, comprising: a nucleic acid molecule containing a nucleotide sequence encoding a recombinant engineered coronavirus spike protein, wherein the nucleic acid molecule is positioned in a multiple cloning site; an intron upstream of the nucleic acid molecule; a cytomegalovirus (CMV) promoter upstream of an intron; a 5′ Inverted Terminal Repeat (5′ ITR) upstream of the CMV promoter, a poly-adenosine tail signal sequence downstream of the nucleic acid molecule; a replication origin sequence downstream of the nucleic acid molecule; a selectable marker sequence downstream of the replication origin sequence; and a 3′ Inverted Terminal Repeat (3′ ITR) downstream of the selectable marker sequence.

Further aspects provide a recombinant engineered coronavirus spike protein, comprising a polypeptide sequence having about 90% identity to FIG. 3E.

In additional aspects, provided here is a composition, comprising a recombinant engineered coronavirus spike protein produced by any of the aforementioned methods, and a pharmaceutically acceptable vehicle (e.g., carrier, diluent, excipient, adjuvant).

Some aspects provide a method for producing a recombinant engineered coronavirus spike protein, comprising: culturing a host cell containing and expressing a first nucleic acid sequence encoding an engineered coronavirus spike protein of FIG. 2A-FIG. 2F and FIG. 3A-FIG. 3R, wherein the culturing step occurs at a first period of time at a first temperature and at a second period of time at a second temperature, and optionally at a third period of time at a third temperature. In some embodiments, a second nucleic acid sequence encoding the transposase DNA is part of a co-transfected vector which does not integrate into the genome of the cells, but transiently provides the transposases that mediate integration of the Spike protein expression cassette into the genome. Such embodiments using the two nucleic acid sequences or vectors are for generating recombinant engineered coronavirus spike protein cell populations before the production phase.

### BRIEF DESCRIPTION OF FIGURES

The novel features of the embodiments are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an illustration of a coronavirus structure with mutations from several SARS-CoV-2 variants and coronavirus spike protein structure comprising S1 and S2 with protein domains.

FIGS. 2A-2F show a table of SARS-CoV-2 variants and spike protein mutations including receptor binding domain mutations relative to a modified sequence of the disclosure (FIG. 3E).

FIGS. 3A-3F show a table containing spike protein sequences for SARS-CoV-2 variants containing additional modifications, including, for example, a furin cleavage site mutation (GSAS (SEQ ID NO: 28)) (i.e., a non-functional furin cleavage site; bold text), a T4 foldon sequence (GYIPEAPRDGQAYVRKDGEWVLLSTFL (SEQ ID NO: 3); underlined text) that replaces a transmembrane domain and an intracellular tail domain. FIGS. 3A-3R disclose SEQ ID NOS 4-7, 2, and 8-20, respectively, in order of appearance.

FIGS. 4A-4M show a sequence alignment of (1) the modified sequence (FIG. 3E); (2) the Delta spike protein (FIG. 3C); (3) the Omicron BA1 spike protein (FIG. 3D); (4) FKS01 (FIG. 3F); (5) FKS02 (FIG. 3G); (6) FKS03 (FIG. 3H); (7) FKS04 (FIG. 3I); (8) FKS05 (FIG. 3J); (9) FKS06 (FIG. 3K); (10) FKS07 (FIG. 3L); (11) FKS08 (FIG. 3M); (12) FKS09 (FIG. 3N); (13) FKS10 (FIG. 3O); (14) FKS11 (FIG. 3P); (15) FKS12 (FIG. 3Q); (16) FKS13 (FIG. 3R). The sequence alignment of FIGS. 4A-4M discloses SEQ ID NOS 2 and 6-20, respectively, in order of appearance.

FIG. 5 shows a phylogenetic tree based on the amino acid sequences of spike proteins of the most relevant SARS-CoV2 variants and those chimeric spike proteins constructed as FrankenSpikes (FKS) antigens. All sequences contain the stabilizing “HexaPro” mutations (F817P, A892P, A899P, A942P, K986P, V987P) and a mutated furin cleavage site (RRAR (SEQ ID NO: 27) 682-685 GSAS (SEQ ID NO: 28)).

FIGS. 6A-6F show a table containing nucleic acid sequence encoding a modified or engineered coronavirus spike protein of the disclosure. FIGS. 6A-6F disclose SEQ ID NOS 21-26, respectively, in order of appearance.

FIG. 7 shows a plasmid map of the 5268 base pair pXLG6 vector used for expressing DNA/gene of interest, typically inserted at the MCS or downstream of the EF-1-alpha intron element (998 . . . 1941 bp). The plasmid map also includes the ITR piggyBac terminal repeat sequences (321 . . . 13 bp/4050 . . . 4299 bp) and mammalian resistance marker for puromycin (Puro-r; 3834 . . . 3235 bp), as well as the bacterial ampicillin resistance marker (Amp-r, 5251 . . . 4391 bp).

FIG. 8 shows a plasmid map of the pXLG5 vector used as the “mobilizing” or “helper” expression vector in co-transfections, where the gene coding for the Piggy Bac transposase enzyme (mPBase) indicates the position of the transposase gene (905 . . . 2686 bp) which is driven by the cytomegalovirus (CMV) promoter (209 . . . 863 bp). This vector also contains a Zeocin resistance marker, including its own promoter (Zeo; 3870 . . . 4244 bp).

FIG. 9 shows the results of a fed-batch culture evaluation of recombinant CHO pools expressing a Beta homotrimer (CVD7), an Alpha/Beta heterotrimer (CVD6/7) and expressing the FKS05 homotrimer. The titers are given for day 7, day 10 and day 12 during the fed-batch culture.

FIG. 10A shows a chromatogram generated by size exclusion chromatography of proteins detected in the supernatants of harvested CHO cells. The arrow indicates the elution position of FKS05.

FIG. 10B shows an enhanced section of the SEC chromatogram where the peak of the FKS05 product indicates a purity of >96%.

FIG. 10C shows three lanes of 100 ng/well of an SDS polyacrylamide gel with a purified derived engineered spike protein, Delta variant. The broad banding is indicative of glycosylation of the proteins in the preparation.

FIGS. 11A-11F show neutralizing antibody titers from mice that were vaccinated intramuscularly on day 1 and day 21 with Sepivac SWE™ adjuvant alone (PBS) or with 5 microgams of spike proteins (FKS01, FKS02, FKS03, FKS04, FKS05, FKS07, FKS12 or with the ancestral spike protein (Wuhan) or with a spike protein from the BA1 virus). One week after the last dose, sera from these immunized mice were tested for neutralizing activity in a pseudovirus infection assay where the pseudoviruses were either expressing Alpha (A), Beta (B), Delta (C), BA.1 (D), BA.2 (E), or BA.5 (F) spikes.

FIG. 12 shows neutralizing, boost or booster antibody titers, tested with a corresponding pseudovirus infection assay. The boost antibody titers were derived from Wuhan (ancestral) spike vaccinated mice that 20 weeks after the last vaccination, had received one additional boost vaccination with a BA.1 spike protein or with the FKS05 spike. FIG. 12 demonstrates strong support that booster vaccinations with a cross reactive FKS antigen provides an effective and prolonged neutralizing titer in animals.

FIG. 13 shows the capacity for antigen production at different scales of operation to produce vaccine doses based on CHO-produced spike proteins from one bioreactor run on a scale of: 1 liter (L), 10 L, 40 L and 200 L. The data are based on Delta-virus-derived spike proteins at a yield of 2.5 g/L, a recovery of 40% to obtain pure antigen preparations, and the assumption of 20 sg of antigen in an adjuvanted vaccine for humans.

### DETAILED DESCRIPTION

Detailed embodiments of the present disclosure are disclosed herein; however, it is to be understood that the disclosed embodiments are merely illustrative of the disclosure that can be embodied in various forms. In addition, each of the examples given in connection with the various embodiments of the disclosure is intended to be illustrative, and not restrictive.

Detailed embodiments of the present disclosure are disclosed here; however, it is to be understood that the disclosed embodiments are merely illustrative of the disclosure that can be embodied in various forms. In addition, each of the examples given in connection with the various embodiments of the disclosure is intended to be illustrative, and not restrictive. The embodiments of the disclosure described here are based on the finding that specific virus variants maintain the overall structure and function (e.g., the trimeric assembly, plus recognition of receptor surfaces on cells for interaction and invasion of host cells), while varying the virus surface in order to avoid immune detection. For example, the methods of producing an engineered coronavirus spike protein (e.g., mammals, human, dogs, cats, horses, cattle) can also apply to producing any other desired engineered protein, including chimeric protein structures. A person of skill in the art would understand based on the teachings here and what is known in the art, how to prepare an expression vector which encodes the desired engineered protein for introduction into an appropriate host cell or host cell population, culturing the host cell under conditions which allow for expression of the desired engineered protein, and isolation of the desired engineered protein, for further use in applications, including but not limited to therapeutic, research, and diagnostic, such as vaccinations against different viral infections.

The present disclosure provides a supply of engineered coronavirus spike protein, including variants, such as, for example, molecular amino acid variants of the coronavirus spike protein, that contain mutations, generated by genetic engineering of mammalian host cells, resulting in an abundant and reproducible supply of the engineered coronavirus spike protein described here. Embodiments of the disclosure can provide to a person of ordinary skill in the art sufficient insight to follow the methods to generate or produce high-level protein expression from high-yielding cells, such as but not limited to, Chinese hamster ovary (CHO) cells, to obtain engineered coronavirus spike protein of, for example, the SARS-CoV-2 virus. Some embodiments are directed to nucleic acid molecules encoding the engineered coronavirus spike proteins described here, pharmaceutical compositions comprising such nucleic acid molecules or the engineered coronavirus spike proteins of the disclosure, and host cells comprising such nucleic acid molecules encoding the engineered coronavirus spike proteins described here. In embodiments of the disclosure, a subject can be prophylactically treated for a coronavirus infection or disease associated with a coronavirus infection, where the subject can include any animal, where an animal can be classified as a mammal, including humans, domestic and farm animals (e.g., horses, cattle), and zoo, sports, or pet animals, such as dogs, cats, and the like. In some embodiments, the subject is a human.

### Coronavirus Spike Protein

In one embodiment, a modified coronavirus spike protein amino acid sequence comprising 1,249 amino acids corresponding to a SARS-CoV-2 spike protein with modifications is provided here. The S1 portion comprises a signal peptide domain, an N-terminal domain (NTD), a receptor binding domain (RBD), and a protease cleavage site mutation rendering the cleavage site non-functional. The S2 portion comprises a fusion peptide sequence (FP), a heptad repeat 1 (HR1), and a heptad repeat 2 (HR2). The modified coronavirus spike protein amino acid sequence does not comprise a transmembrane domain (TD) nor an intracellular tail. Instead, the modified coronavirus spike protein amino acid sequence comprises a T4 foldon motif also known as a T4 fibritin trimerization foldon motif. Some embodiments of the disclosure provide a protein based on an engineered coronavirus spike protein that comprises, consists essentially of, or consists of the modified coronavirus spike protein described here. In some embodiments, a protein disclosed here comprises an engineered coronavirus spike protein having an amino acid sequence of at least 90% (e.g., 91%, 92%, 93%, 94%, 95%, 96%, 97%, 98%, 99%, 100%) identity to a modified sequence (e.g., modified coronavirus spike protein sequence) of:

(SEQ ID NO: 1)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QCANRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG.

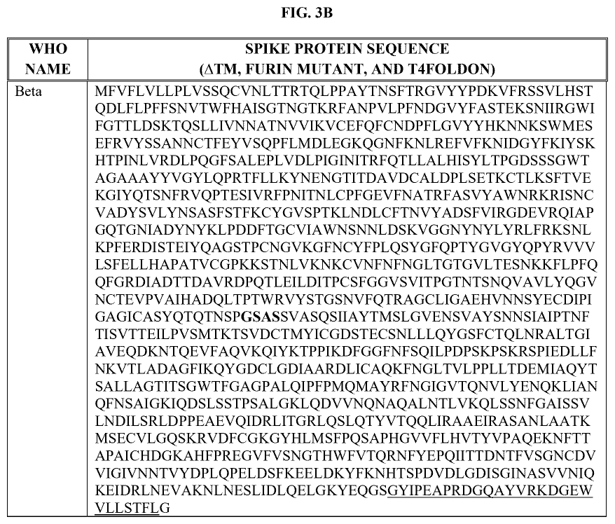

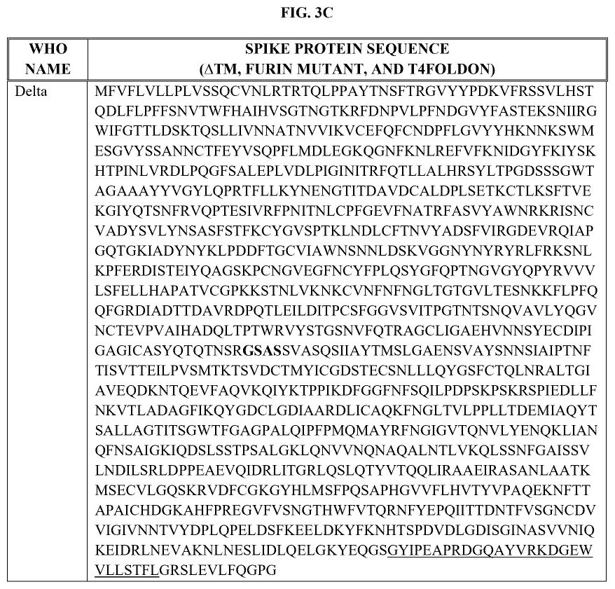

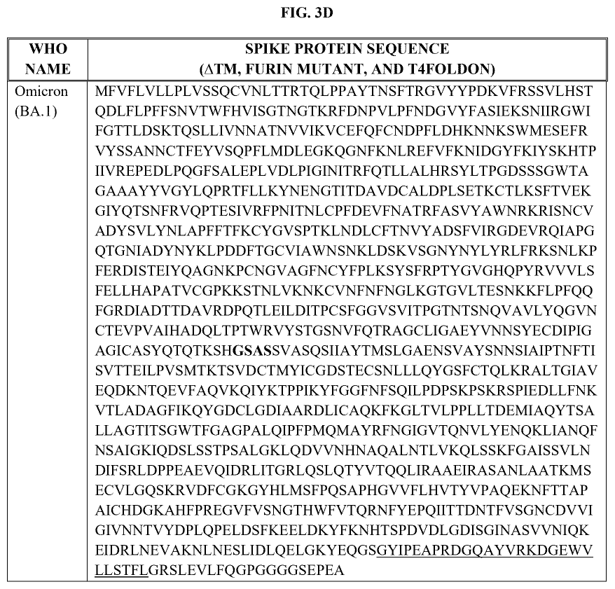

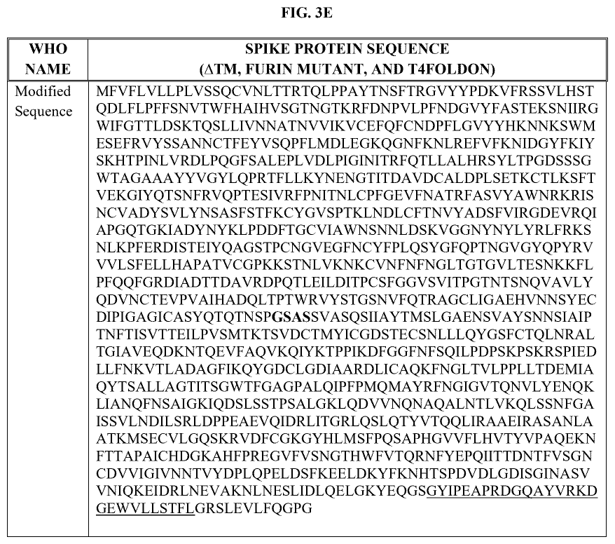

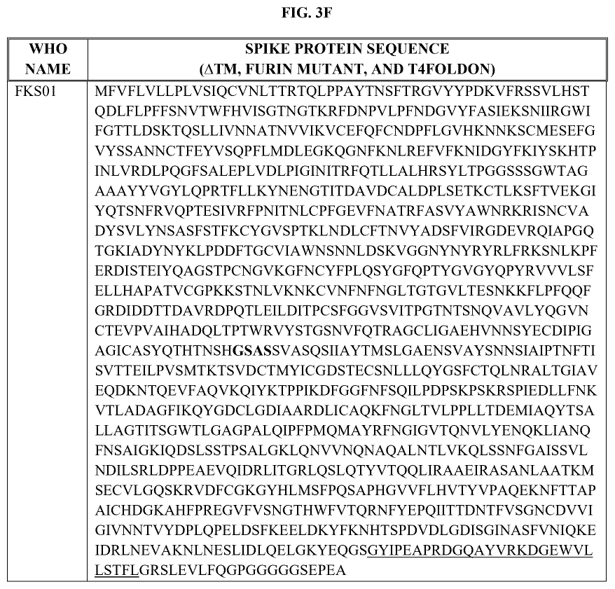

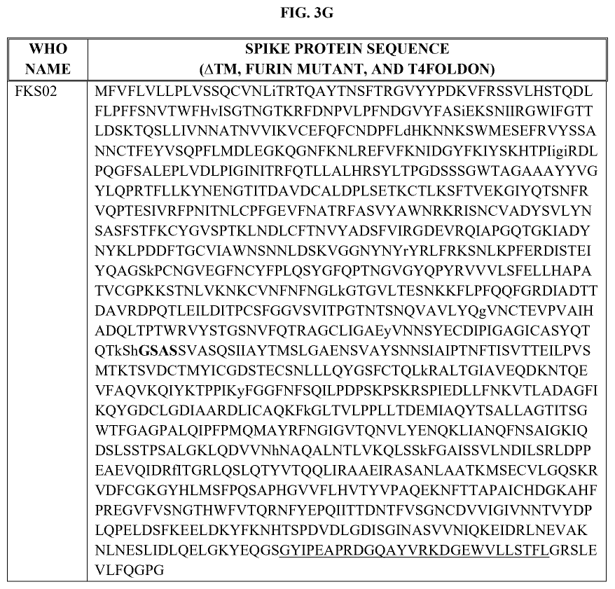

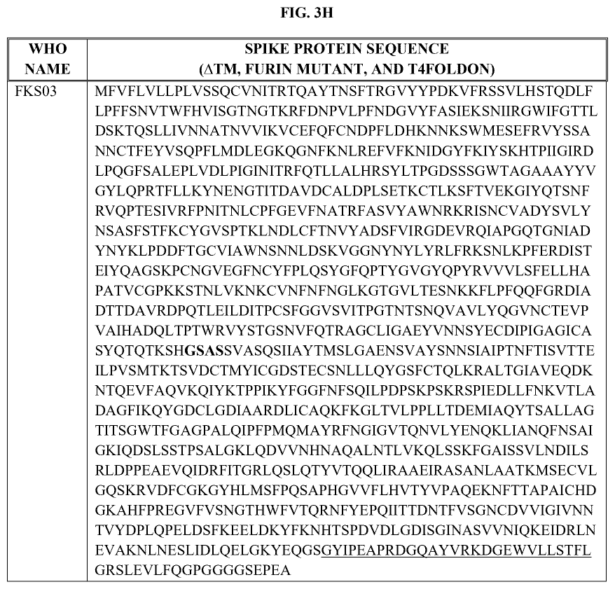

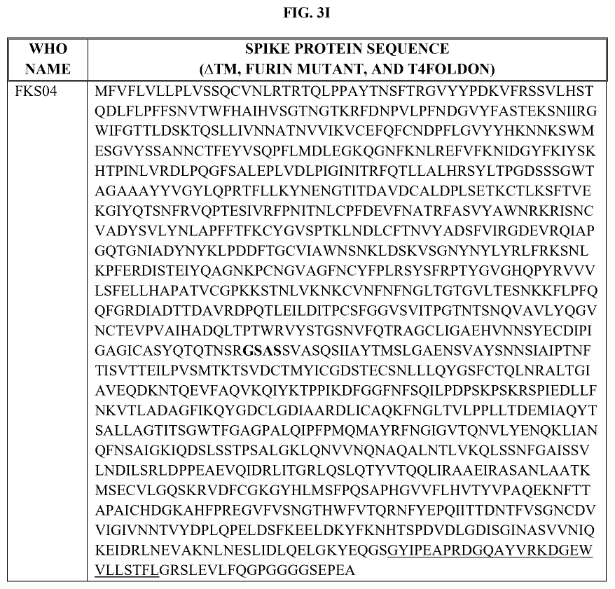

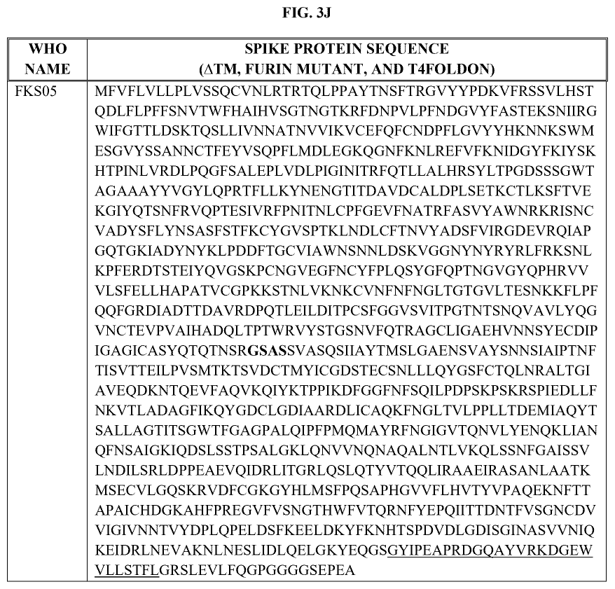

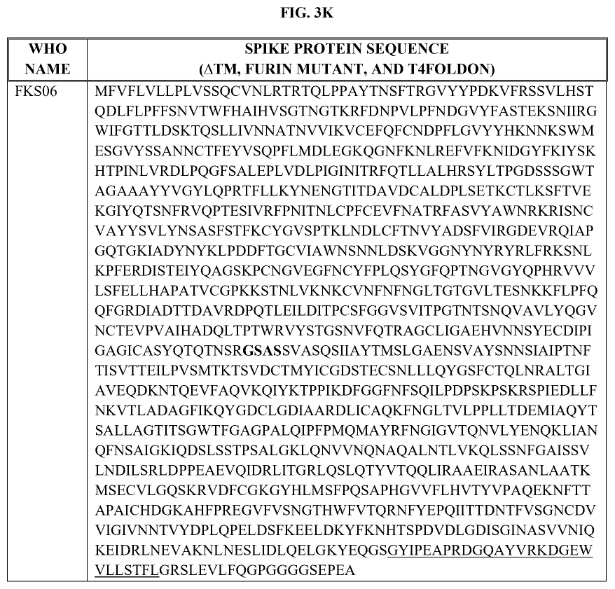

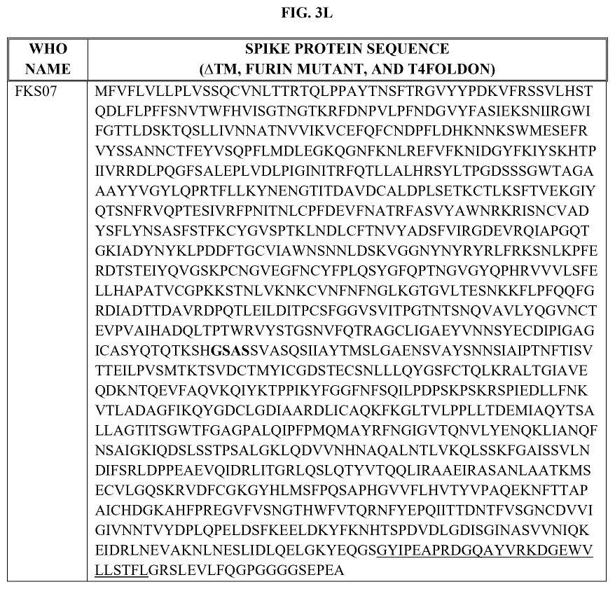

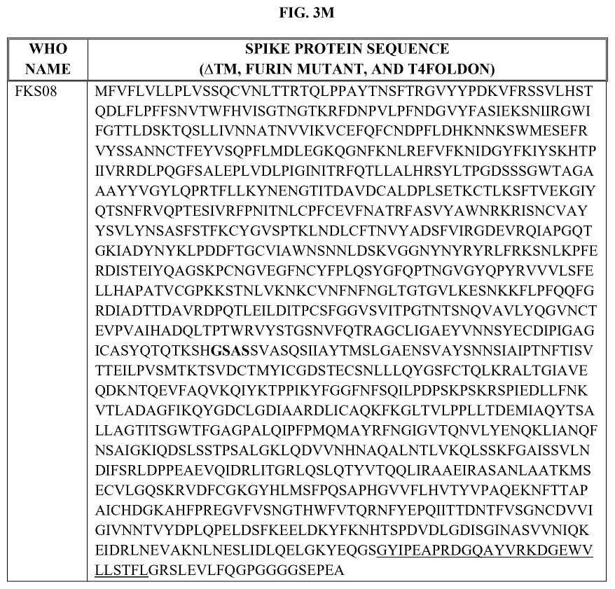

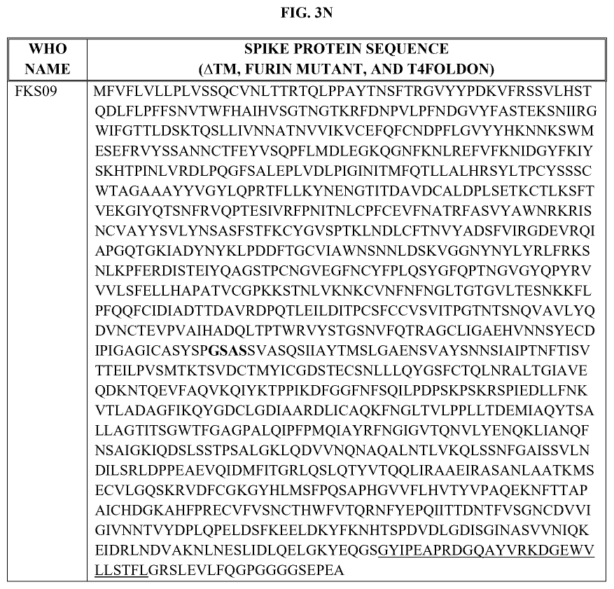

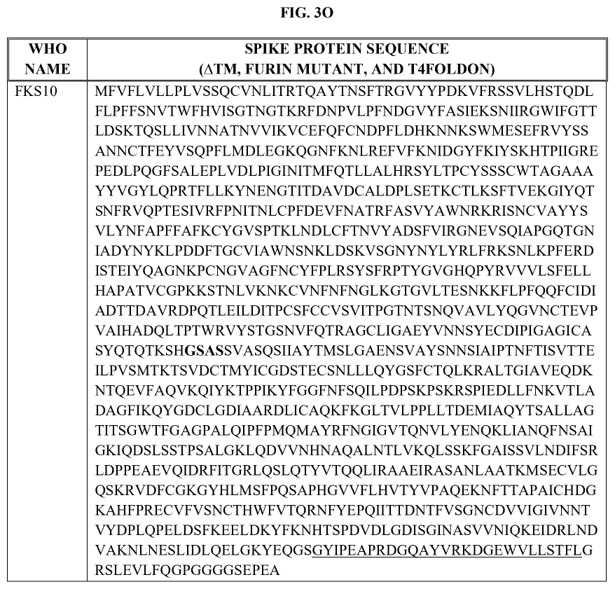

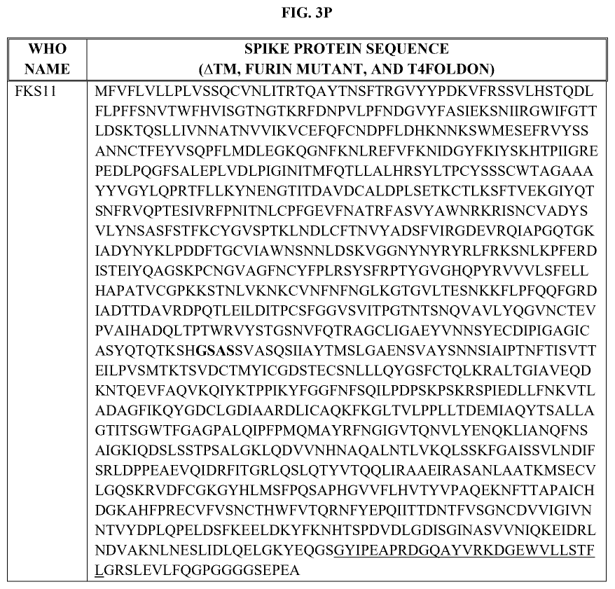

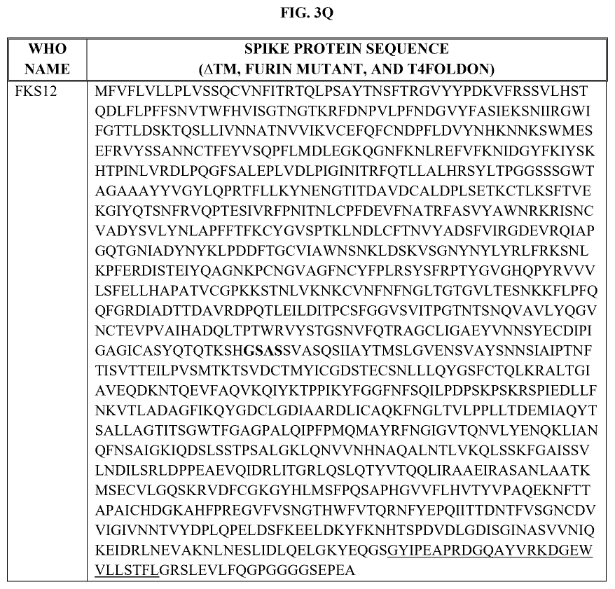

See, FIG. 3E; bold text (furin cleavage site mutation); underlined text (T4 foldon sequence). In some embodiments, a protein comprising an engineered coronavirus spike protein having an amino acid sequence of at least 90% (e.g., 91%, 92%, 93%, 94%, 95%, 96%, 97%, 98%, 99%, 100%) identity to a modified sequence (e.g., modified coronavirus spike protein sequence) of FIG. 3E, comprises, consists essentially of, or consists of: (i) a non-functional protease cleavage site at amino acid positions 682-685 relative to the modified amino acid sequence of FIG. 3E (e.g., a non-functional furin cleavage site of amino acid sequence GSAS (SEQ ID NO: 28)); (ii) a T4 foldon sequence (e.g., GYIPEAPRDGQAYVRKDGEWVLLSTFL (SEQ ID NO: 3) at amino acid positions 1211-1237 relative to the modified amino acid sequence of FIG. 3E) that replaces a transmembrane domain and an intracellular tail of a wild-type or unmodified coronavirus spike protein; and (iii) proline at positions 817, 892, 899, 942, 986, 987 (i.e., P817, P892, P899, P942, P986, and P987) relative to the modified sequence of FIG. 3E.

The engineered coronavirus spike protein of the disclosure is designed or engineered to induce cross-reactive immunity when injected into animals or humans, such as mice, rabbits, non-human primates or humans to produce an engineered coronavirus spike protein described here. Additionally, the engineered coronavirus spike protein is designed to incorporate modifications that increase manufacturability and stability. The engineered coronavirus spike protein is based on such a modified coronavirus spike protein amino acid sequence. The coronavirus spike protein can recall or combine mutations from the various coronavirus variants considered variants of concern and/or variants of interest, for example, Wuhan/Alpha, Delta, Omicron, and their subvariants. The coronavirus spike protein can also comprise sequences from coronaviruses different from the SARS-CoV2 virus, such as from other members of the family of beta coronaviruses, such as SARS or MERS virus and other related viruses, even from the non-human animal kingdom. In so doing, the engineered coronavirus spike proteins of the disclosure can be used as an antigen in a universal, broadly protective, cross-reactive coronavirus vaccine to prevent coronavirus infection despite the appearance of mutating viral variants, where the vaccine inducing high antibody titers against multiple coronavirus variants, for example, Wuhan/Alpha, Delta, Omicron, and any subvariants thereof. Large-scale production of such engineered coronavirus spike proteins from stable, clonally-derived cell populations can provide sufficient antigen for hundreds of millions of antigen doses at a lower cost than any of the existing vaccines available against coronaviruses, e.g., SARS-CoV-2. The large-scale, high-throughput approach to modified viral antigen production described here allows for the rapid production of vaccines to combat viral infections, such as for example, SARS-CoV-2 infection or COVID-19 disease caused by SARS-CoV-2, as well as infections other than coronavirus infections, where mass quantities of vaccines are necessary to deter the spread of infections during, for example, a global pandemic, and treat infected individuals.

Some embodiments provide the engineered coronavirus spike protein of the disclosure that comprises at least one mutation (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) to the modified coronavirus amino acid sequence (see, FIG. 3E), where the mutation is selected from the group consisting of: 0 or more mutations (e.g., 0-1 mutation) of a signal peptide sequence (1-13 aa); 3 or more mutations (e.g., 3-16 mutations) of an N-terminal domain (13-305 aa); 2 or more mutations (e.g., 2-19 mutations) of a receptor binding domain (319-541aa); 2 or more mutations (e.g., 2-10 mutations) of a receptor binding motif (437-508 aa); 0 or more mutations (e.g., 0-1 mutation) of a fusion peptide sequence (788-806 aa); 0 or more mutations (e.g., 0-3 mutations) of a heptad repeat 1 (912-984 aa); 0 or more mutations (e.g., 0-1 mutation) of a heptad repeat 2 (1163-1213 aa); and any combinations thereof. Engineered coronavirus spike protein modifications can incorporate spike protein mutations found in multiple coronavirus variants for the production of neutralizing antibodies. The modifications described here also incorporate coronavirus spike protein mutations that facilitate stability and manufacturability.

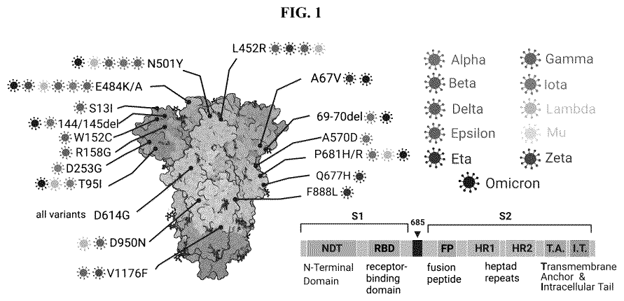

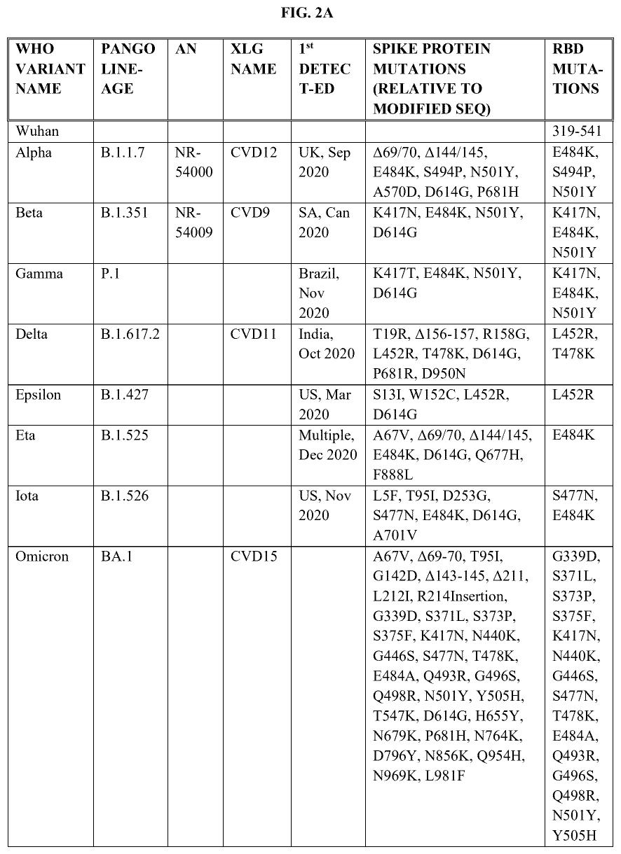

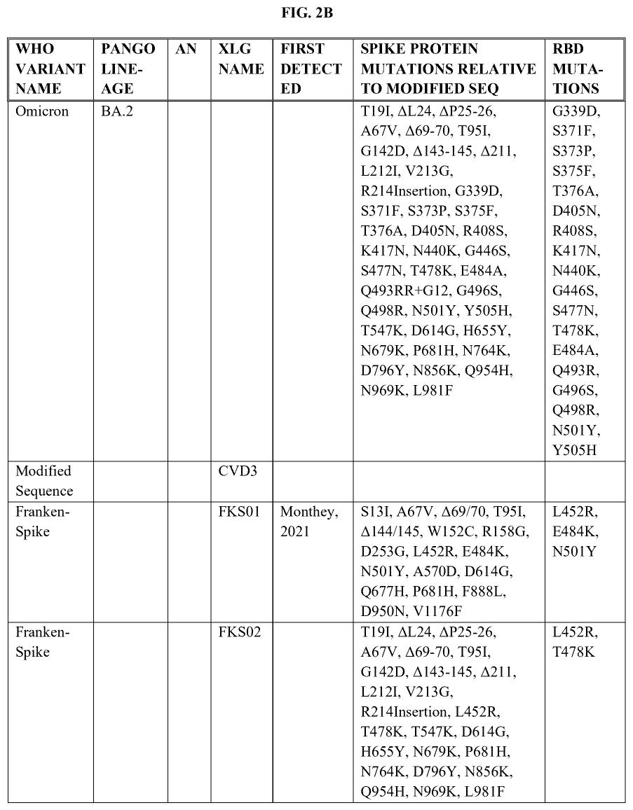

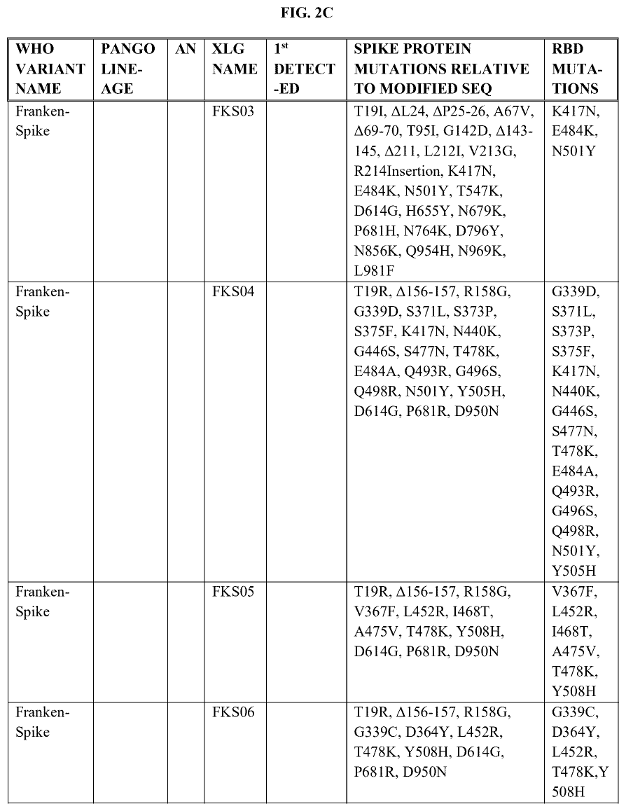

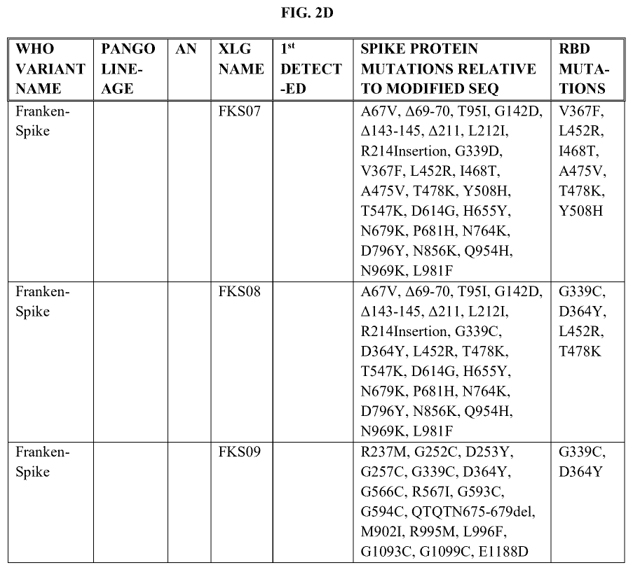

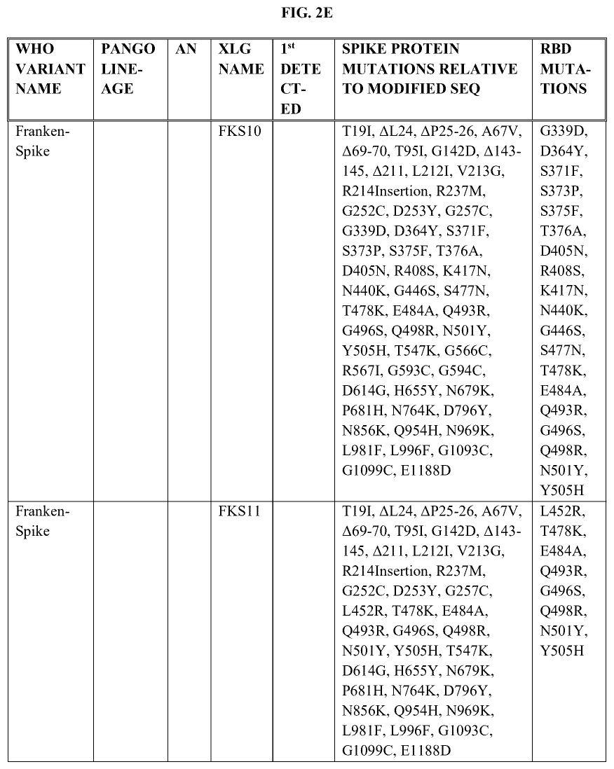

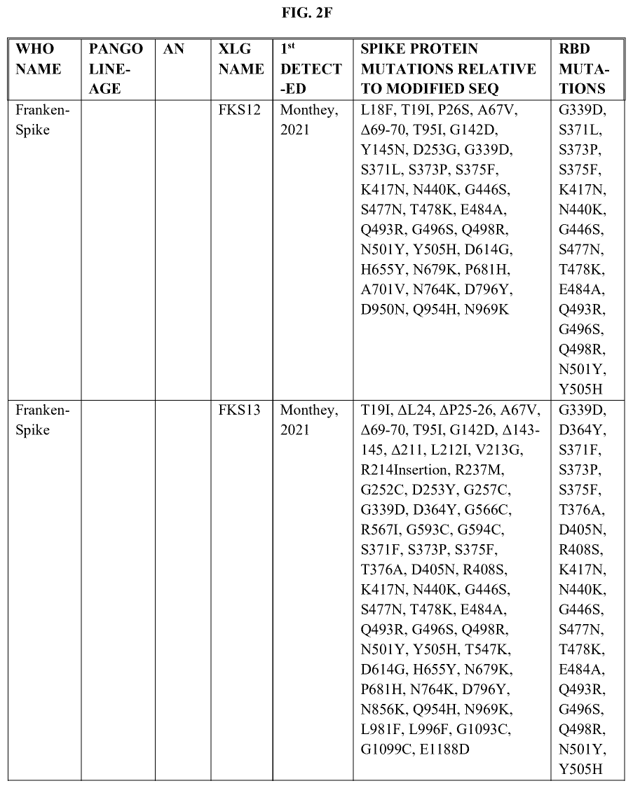

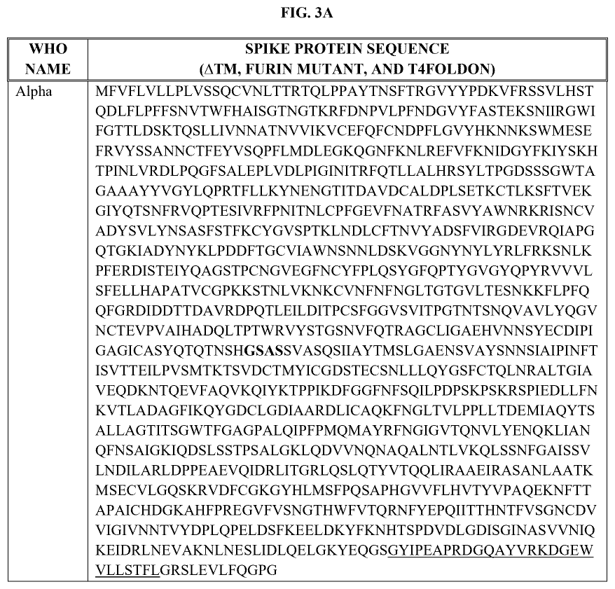

In some embodiments, the protein comprising an engineered coronavirus spike protein such as an engineered severe acute respiratory syndrome coronavirus 2 (SARS-CoV-2) spike protein or variants thereof incorporating mutations found in a multitude of SARS-CoV-2 variants. FIG. 1 illustrates known variants and their mutations. FIGS. 2A-2F; and FIGS. 3A-3R show various SARS-CoV-2 variants, mutations based on the modified sequence of the disclosure, and sequences thereof. Such mutations shown in FIG. 1 and FIGS. 2A-2F can be incorporated into an engineered coronavirus spike protein described here.

Additional embodiments provide the engineered coronavirus spike protein described here that comprises at least one spike protein mutation. Further embodiments can include the engineered coronavirus spike protein of the disclosure comprising at least one mutation in a recombinant binding domain. In other embodiments, the engineered coronavirus spike protein of the disclosure comprises, consists essentially of, or consists of an amino acid sequence of at least 90% (e.g., 91%, 92%, 93%, 94%, 95%, 96%, 97%, 98%, 99%, 100%) identity to a modified sequence of:

(SEQ ID NO: 1)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QCANRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG

(see, FIG. 3E), where the engineered coronavirus spike protein comprises at least one mutation (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) relative to the modified sequence, where the mutation is selected from the group consisting of: T19I; deletion of L24; deletion of P25-P26; deletion of V143; deletion of Y144-Y145; Y145N; deletion of E156; deletion of F157; R158G; deletion of N211; L212I; V213G; insertion of R214; R237M; G252C; D253Y; G257C; G339D; D364Y; V367F; S371L; S373P; S375F; T376A; D405N; R408S; N440K; G446S; I468T; A475V; S477N; E484A; Q493R; G496S; Q498R; Y505H; Y508H; T547K; G566C; R567I; A570D; G593C; G594C; deletion of: Q675, T676, Q677, T678, and N679; N679K; N764K; D796Y; N856K; M902I; Q954H; N969K; L981F; R995M; L996F; G1093C; G1099C; E1188D; and any combinations thereof.

In some embodiments, a protein comprising the engineered coronavirus spike protein comprises an S1 portion and an S2 portion with modifications for stability and/or manufacturability. The S1 portion comprises a signal peptide domain, an N-terminal domain, a receptor-binding domain, and a non-functional protease cleavage site at amino acid positions 682-685 relative to the modified amino acid sequence of FIG. 3E (e.g., non-functional furin cleavage site; GSAS (SEQ ID NO: 28)). Non-limiting examples of proteases include FURIN, TMPRSS2, and Cathepsins. The S2 portion comprises a fusion peptide sequence, a heptad repeat 1, and a heptad repeat 2, without a transmembrane domain and an intracellular tail. A T4 foldon sequence (GYIPEAPRDGQAYVRK DGEWVLLSTFL (SEQ ID NO: 3)) replaces the transmembrane domain and intracellular tail at amino acid positions 1211-1237 relative to the modified amino acid sequence of FIG. 3E. The engineered coronavirus spike protein also includes proline at positions 817, 892, 899, 942, 986, 987 (i.e., P817, P892, P899, P942, P986, and P987) relative to the modified sequence of FIG. 3E for stability and/or manufacturability. However, the coronavirus spike protein is inherently unstable. Therefore, additional mutations are selected to provide improved stability over an unmodified coronavirus spike protein. Mutations are also selected to result in improved manufacturability providing large quantities of the engineered coronavirus spike protein for use as, for example, an antigen in vaccines. Some embodiments are directed to a protein comprising an engineered coronavirus spike protein having a ratio of mutations in the S1 portion to the mutations in the S2 portion relative to the modified sequence described here (see, FIG. 3E), where the ratio can be selected from the group consisting of: 9/6 or greater, 20/1 or less; and a range of 9/6-20/1 (e.g., 11/6; 17/6; 29/10; 19/6; 20/6; 44/10; 28/6; 15/3; 10/1; 11/1; 20/1).

Additional embodiments provide a protein comprising an engineered coronavirus spike protein that comprises at least one mutation (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) relative to the modified amino acid sequence (see, FIG. 3E), where the mutations are selected from:
(a) deletion of: Y144 and Y145; and substitution of R158G and A570D; or
(b) T19I; deletion of L24; deletion of: P25 and P26; deletion of V143; deletion of: Y144 and (c) Y145; deletion of N211; substitution of L212I; V213G; insertion of R214; T547K; N679K; N764K; D796Y; N856K; Q954H; N969K; L981F; or
(d) T19I; deletion of L24; deletion of: P25 and P26; deletion of V143; deletion of: Y144 and Y145; deletion of N211; L212I; V213G; insertion of R214; T547K; N679K; N764K; D796Y; N856K; Q954H; N969K; L981F; or
(e) deletion of E156; deletion of F157; R158G; G339D; S371L; S373P; S375F; N440K; G446S; S477N; E484A; Q493R; G4% S; Q498R; Y505H; or
(f) deletion of E156; deletion of F157; R158G; V367F; I468T; A475V; Y508H; or
(g) deletion of E156; deletion of F157; R158G; G339C; D364Y; Y508H; or
(h) deletion of V143; deletion of: Y144 and Y145; deletion of N211; L212I; insertion of R214; G339D; V367F; I468T; A475V; Y508H; T547K; N679K; N764K; D796Y; N856K; Q954H; N969K; L981F; or
(i) deletion of V143; deletion of: Y144 and Y145; deletion of N211; L212I; insertion of R214; G339C; D364Y; T547K; N679K; N764K; D796Y; N856K; Q954H; N969K; L981F; or
(j) R237M; G252C; D253Y; G257C; G339C; D364Y; G566C; R567I; G593C; G594C; deletion of: Q675, T676, Q677, T678, N679; M902I; R995M; L996F; G1093C; G1099C; E1188D; or
(k) T19I; deletion of L24; deletion of: P25 and P26; deletion of V143; deletion of: Y144 and Y145; deletion of N211; L212I; V213G; insertion of R214; R237M; G252C; D253Y; G257C; G339D; D364Y; S371F; S373P; S375F; T376A; D405N; R408S; N440K; G446S; S477N; E484A; Q493R; G4% S; Q498R; Y505H; T547K; G566C; R567I; G593C; G594C; N679K; N764K; D796Y; N856K; Q954H; N969K; L981F; L996F; G1093C; G1099C; E1188D; or
(l) T19I; deletion of L24; deletion of: P25 and P26; deletion of V143; deletion of: Y144 and Y145; deletion of N211; L212I; V213G; insertion of R214; R237M; G252C; D253Y; G257C; E484A; Q493R; G496S; Q498R; Y505H; T547K; N679K; N764K; D796Y; N856K; Q954H; N969K; L981F; L996F; G1093C; G1099C; E1188D; or
(m) T19I; deletion of P26; Y145N; G339D; S371F; S373P; S375F; N440K; G446S; S477N; E484A; Q493R; G4% S; Q498R; Y505H; N679K; N764K; D796Y; Q954H; N969K.

In another embodiment, a protein of the disclosure, comprising an engineered coronavirus spike protein can comprise an amino acid sequence of at least 90% identity (e.g., 91%, 92%, 93%, 94%, 95%, 96%, 97%, 98%, 99%, 100%) to a modified sequence of:

(SEQ ID NO: 1)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QCANRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG
(see, FIG. 3E),

where the engineered coronavirus spike protein comprises a plurality of mutations (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) relative to the modified sequence (see, FIG. 3E), wherein the mutations are selected from:
(a) S13I; A67V; deletion of H69 and V70; T95I; deletion of Y144 and Y145; W152C; R158G; D253G; L452R; E484K; N501Y; A570D; D614G; Q677H; P681H; F888L; D950N; V1176F; or
(b) T19I; deletion of L24, P25, and P26; A67V; deletion of H69 and V70; T95I; G142D; deletion of V143; deletion of: Y144 and Y145; deletion of N211; L212I; V213G; insertion of R214; L452R; T478K; T547K; D614G; H655Y; N679K; P681H; N764K; D796Y; N856K; Q954H; N969K; L981F; or
(c) T19I; deletion of L24, P25, and P26; A67V; deletion of H69 and V70; T95I; G142D; deletion of V143; deletion of: Y144 and Y145; deletion of N211; L212I; V213G; insertion of R214; K417N; E484K; N501Y; T547K; D614G; H655Y; N679K; P681H; N764K; D796Y; N856K; Q954H; N969K; L981F; or
(d) T19R; deletion of E156; deletion of F157; R158G; G339D; S371L; S373P; S375F; K417N; N440K; G446S; S477N; T478K; E484A; Q493R; G496S; Q498R; N501Y; Y505H; D614G; P681R; D950N; or
(e) T19R; deletion of E156; deletion of F157; R158G; V367F; L452R; I468T; A475V; T478K; Y508H; D614G; P681R; D950N; or
(f) T19R; deletion of E156; deletion of F157; R158G; G339D; D364Y; L452R; T478K; Y508H; D614G; P681R; D950N; or
(g) A67V; deletion of H69 and V70; T95I; G142D; deletion of V143; deletion of: Y144 and Y145; deletion of N211; L212I; insertion of R214; G339D; V367F; L452R; I468T; A475V; T478K; Y508H; T547K; D614G; H655Y; N679K; P681H; N764K; D796Y; N856K; Q954H; N969K; L981F; or
(h) A67V; deletion of H69 and V70; T95I; G142D; deletion of V143; deletion of: Y144 and Y145; deletion of N211; L212I; insertion of R214; G339C; D364Y; L452R; T478K; T547K; D614G; H655Y; N679K; P681H; N764K; D796Y; N856K; Q954H; N969K; L981F; or
(i) R237M; G252C; D253Y; G257C; G339C; D364Y; G566C; R567I; G593C; G594C; deletion of: Q675, T676, Q677, T678, N679; M902I; R995M; L996F; G1093C; G1099C; E1188D; or
(j) T19I; deletion of L24; deletion of: P25 and P26; A67V; deletion of: H69 and V70; T95I; G142D; deletion of V143; deletion of: Y144 and Y145; deletion of N211; L212I; V213G; insertion of R214; R237M; G252C; D253Y; G257C; G339D; D364Y; S371F; S373P; S375F; T376A; D405N; R408S; K417N; N440K; G446S; S477N; T478K; E484A; Q493R; G496S; Q498R; Y505H; T547K; G566C; R567I; G593C; G594C; D614G; H655Y; N679K; P681H; N764K; D796Y; N856K; Q954H; N969K; L981F; L996F; G1093C; G1099C; E1188D; or
(k) T19I; deletion of L24; deletion of: P25 and P26; A67V; deletion of: H69 and V70; T95I; G142D; deletion of V143; deletion of: Y144 and Y145; deletion of N211; L212I; V213G; insertion of R214; R237M; G252C; D253Y; G257C; L452R; T478K; E484A; Q493R; G496S; Q498R; N501Y; Y505H; T547K; D614G; H655Y; N679K; P681H; N764K; D796Y; N856K; Q954H; N969K; L981F; L996F; G1093C; G1099C; E1188D; or
(l) L18F; T19I; P26S; A67V; deletion of: H69 and V70; T95I; G142D; Y145N; D253G; G339D; S371L; S373P; S375F; K417N; N440K; G446S; S477N; T478K; E484A; Q493R; G4% S; Q498R; N501Y; Y505H; D614G; H655Y; N679K; P681H; A701V; N764K; D796Y; D950N; Q954H; N969K.

In some embodiments, the engineered coronavirus spike protein comprises a non-functional protease cleavage site. Another embodiment can be directed to an active engineered coronavirus spike protein described here that comprises a protease inhibition activity, where the protease is, for example, furin. For example, the engineered coronavirus spike protein can comprise a furin cleavage site comprising an amino acid sequence of RRAR (SEQ ID NO: 27) that has been mutated to form a non-functional furin cleavage site (GSAS (SEQ ID NO: 28)), thereby preventing the cleavage into separate S1 and S2 portions. The non-functional furin cleavage site can comprise an amino acid sequence of GSAS (SEQ ID NO: 28) at positions 682-685 relative to the modified amino acid sequence of FIG. 3E, which can increase expression, trimer assembly, stability, or combinations thereof. Further embodiments provide the engineered coronavirus spike protein that does not comprise a transmembrane domain nor an intracellular tail of the S2 portion. In further embodiments, the engineered coronavirus spike proteins of the disclosure comprise proline at positions 817, 892, 899, 942, 986, 987 (i.e., P817, P892, P899, P942, P986, and P987) relative to the modified sequence of FIG. 3E. Additional embodiments are directed to the disclosed engineered coronavirus spike protein that comprises a T4 foldon motif (e.g., GYIPEAPRDGQAYVRKDGEWVLLSTFL (SEQ ID NO: 3) at amino acid positions 1211-1237 relative to the modified amino acid sequence of FIG. 3E; T4 fibritin trimerization foldon motif), which replaces the transmembrane domain and intracellular tail at the carboxy terminal end of the spike protein.

In some embodiments, a leader sequence (i.e., secretory signal peptide sequence) of the wild type coronavirus spike protein sequence can be maintained or replaced with a different leader sequence, such as but not limited to, the leader sequence of a human heavy chain IgG sequence, a human serum albumin leader sequence, a coronavirus spike protein leader sequence, a mouse Ig Kappa light chain leader sequence, and others. Alternative leader sequences which can be incorporated into the coronavirus spike protein sequence. Another leader sequence is the “natural” coronavirus spike protein leader sequence. The presence and location of signal peptide cleavage can be predicted and compared for the “natural” coronavirus spike protein leader sequence as well as the other leader sequences by the SignalP 4.1 program (H. Nielsen. Methods Mol. Biol. 1611:59-73, 2017. doi: 10.1007/978-1-4939-7015-5_6). The leader sequence or signal sequence can be cleaved off before secretion from the cells. Including a natural coronavirus spike protein leader peptide sequence of 13 amino acids, a modified coronavirus spike protein amino acid sequence described here can comprise about 1,249 amino acids. (See, e.g., FIG. 3E). Alternatively, other leader peptide sequences of varying lengths can be substituted in the coronavirus spike protein sequence. In one embodiment, the leader sequence can have at least about 10 residues, at least about 11 residues, at least about 12 residues, at least about 13 residues, at least about 14 residues, at least about 15 residues, at least about 19 residues, or at least about 24 residues. Another embodiment can be directed to a leader sequence that is from the same species as the desired coronavirus spike protein, for example, leader sequence and coronavirus spike protein. A further embodiment can be directed to a leader sequence that is cleaved before secretion.

In one embodiment, an engineered coronavirus spike protein thereof is an active protein that is about 90% to about 100% free of or essentially free of contaminants, such as but not limited to, non-human components, animal components, or human components which induce an undesirable immune response. The engineered coronavirus spike protein described herein can have a purity of about 90% to about 100%, about 95% to about 99.9%, or about 98% to about 99%; a purity of greater than about 90%, greater than about 95%, greater than about 98%, greater than about 99%, or greater than about 99.9%; or a purity of about 95%, of about 96%, of about 97%, of about 98%, about 99%, about 99.9%, or about 100%.

An engineered coronavirus spike protein that maintains its cross-reactivity inducing an immune response and activity of inhibiting protease activity, any sequence of the modified amino acid sequence shown in FIG. 3E can be modified by at least one of: a substitution, an insertion, or a deletion as described here. For example, the engineered coronavirus spike protein amino acid sequence can include an amino acid sequence having an amino acid sequence identity of about 90% or greater, about 95% or greater, about 96% or greater, about 97% or greater, about 98% or greater, or about 99% or greater, compared to the modified amino acid sequences of FIG. 3E. In some embodiments, an engineered coronavirus spike protein can be selected from the group consisting of: the modified coronavirus spike protein amino acid sequence of FIG. 3E, any of the engineered coronavirus spike protein sequences provided in FIG. 3, for example, FIG. 3A-FIG. 3R, including those engineered coronavirus spike proteins derived from the modified amino acid sequence of FIG. 3E containing additional modifications and named FrankenSpikes (FKS) (see, FIG. 2A-FIG. 2F;

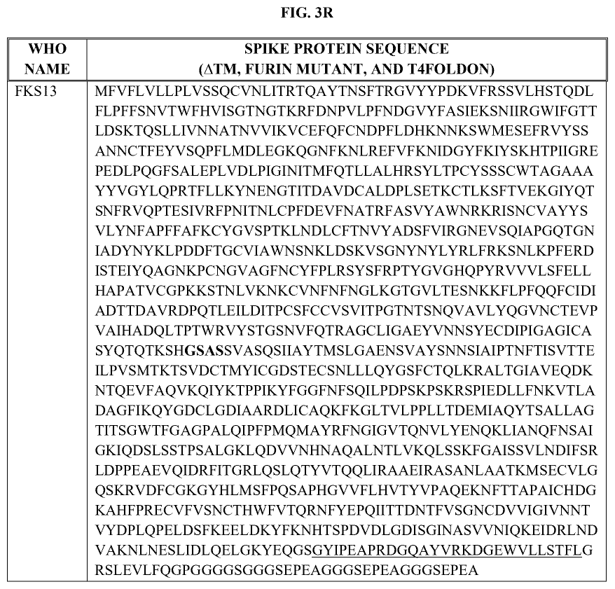

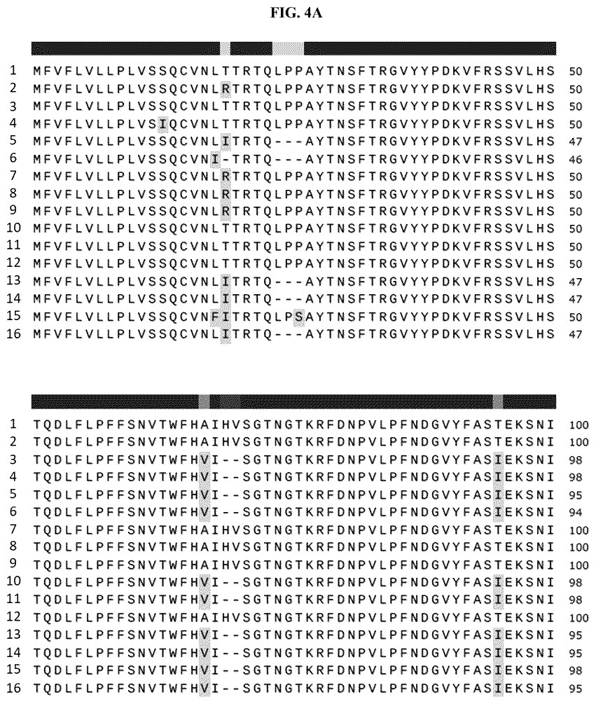

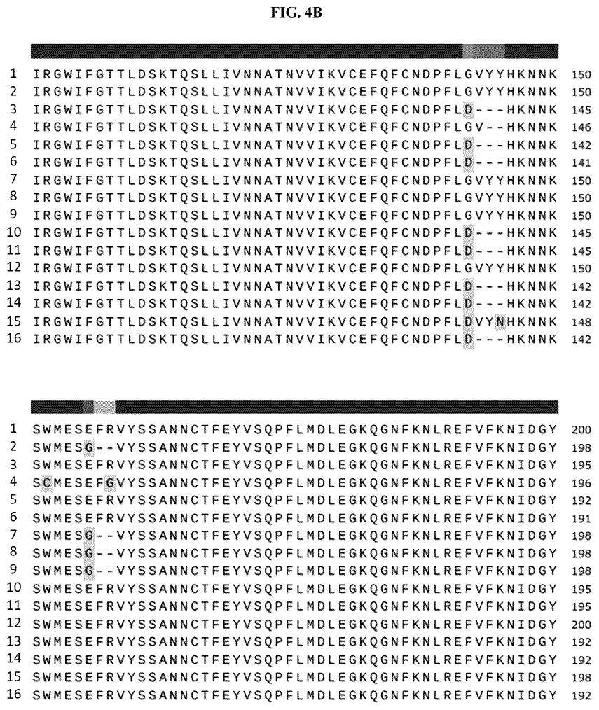

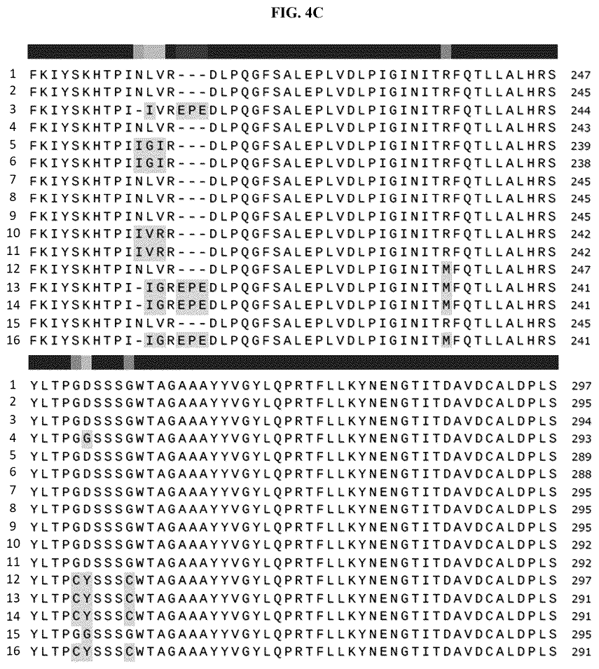

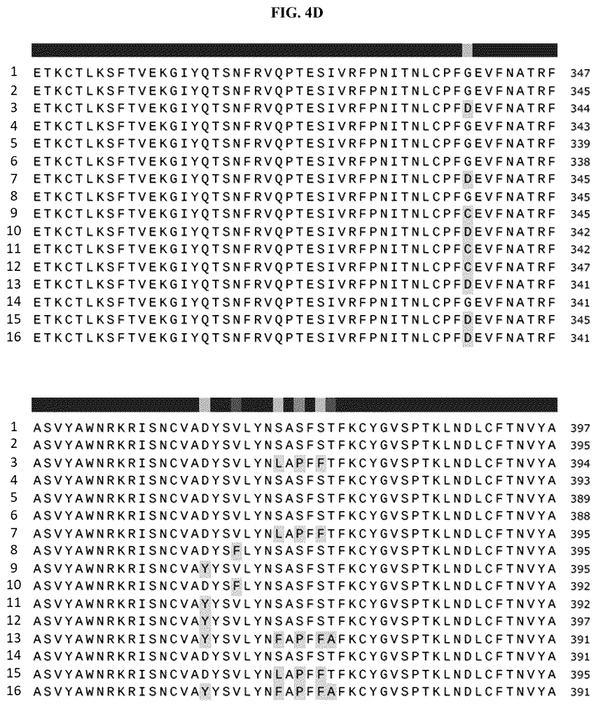

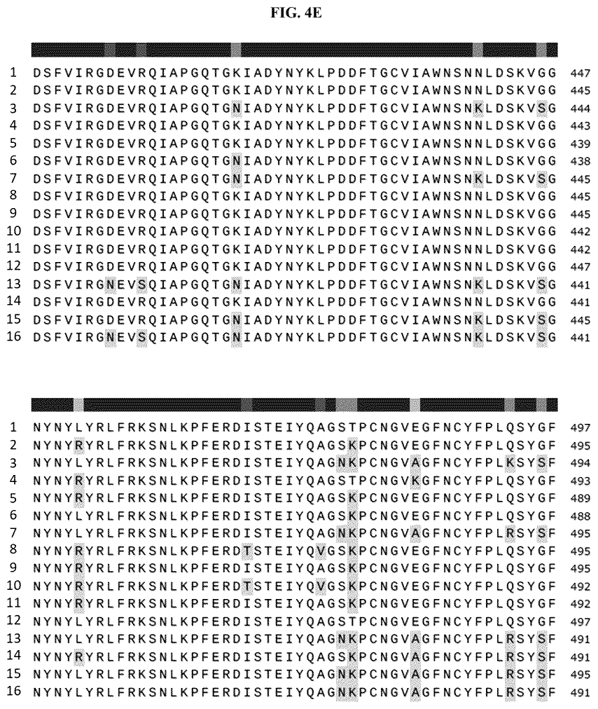

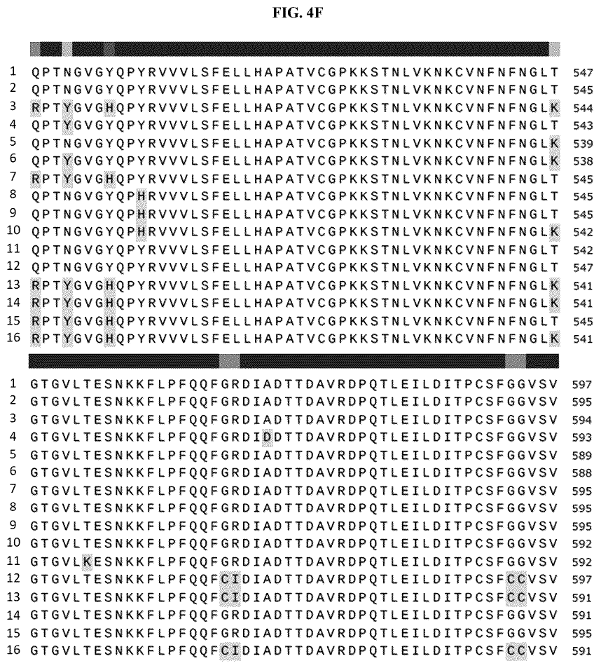

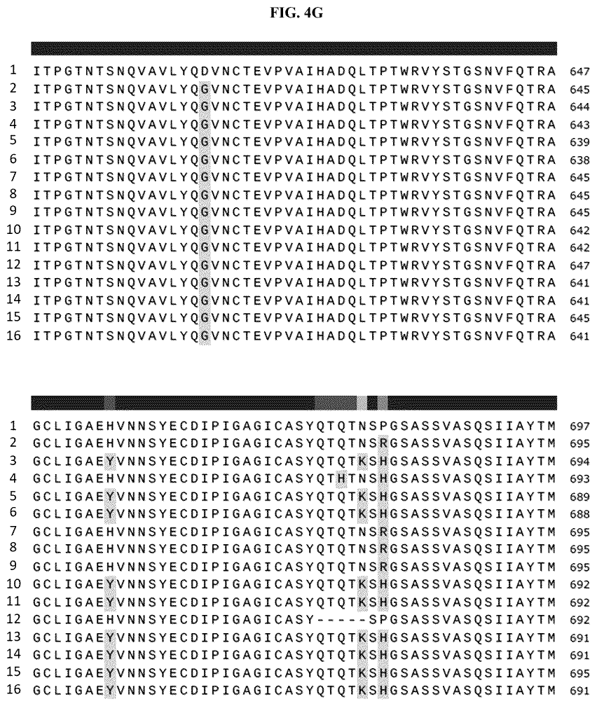

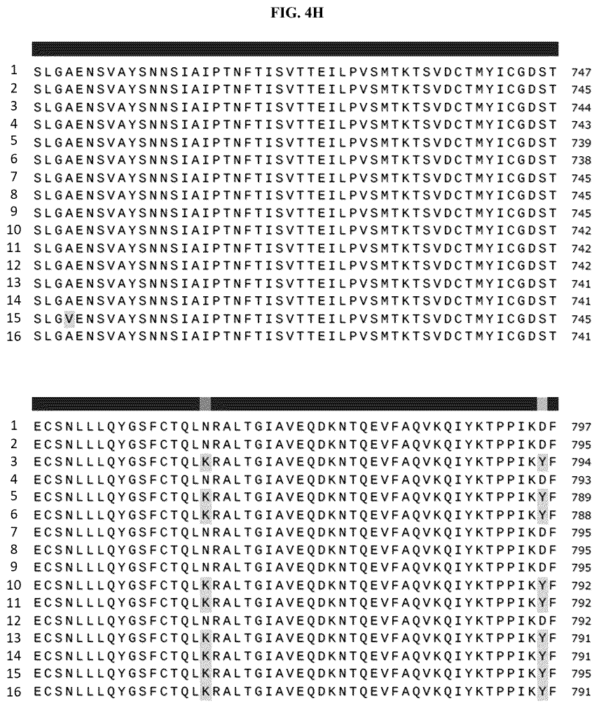

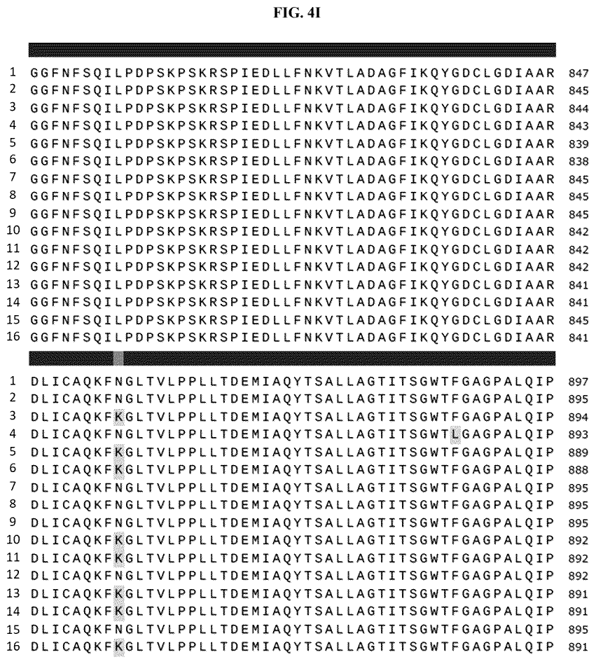

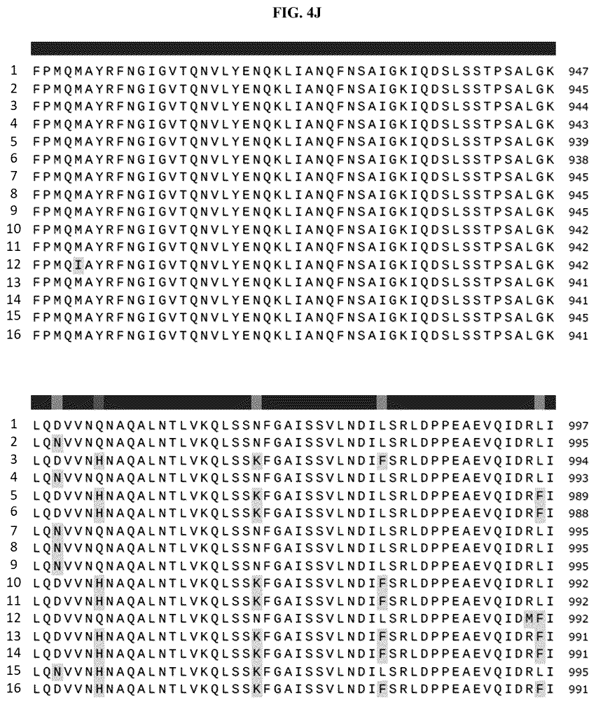

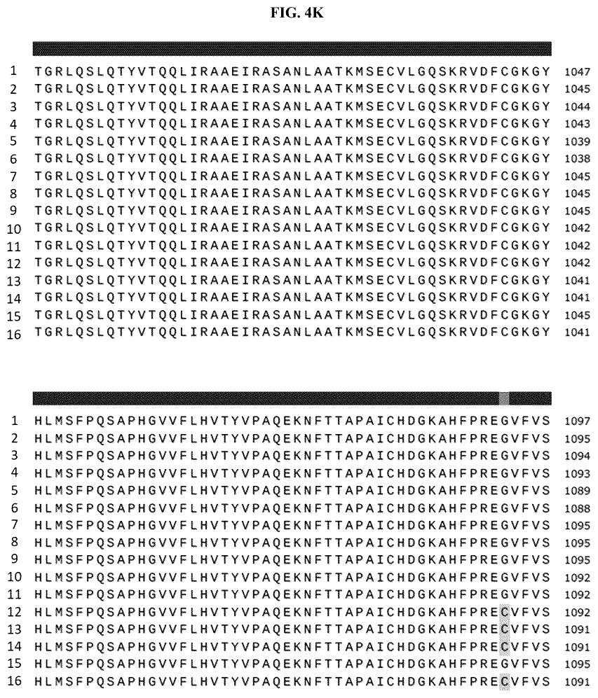

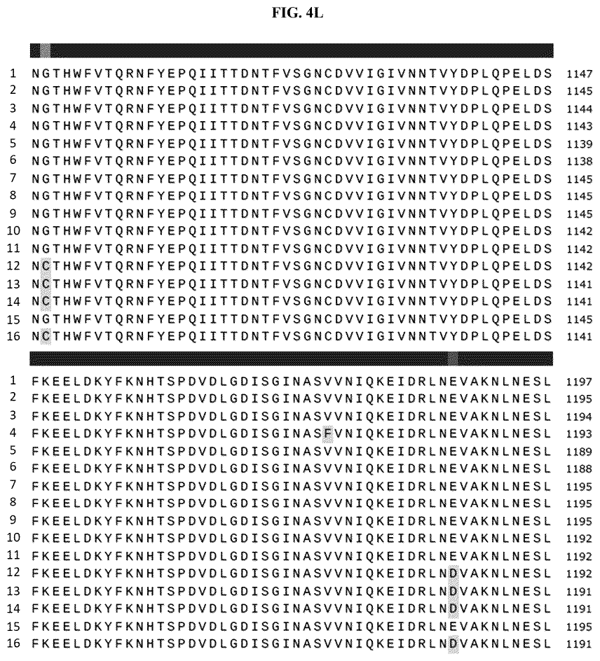

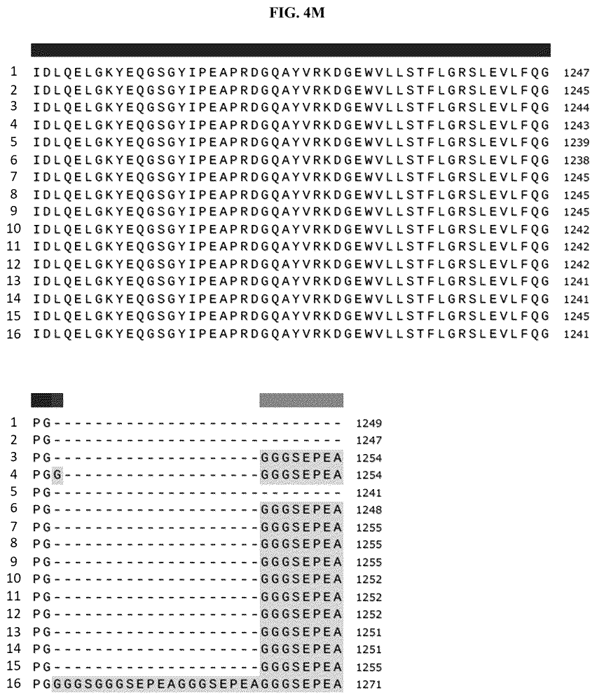

FIG. 3A-FIG. 3R)). FIG. 4A-FIG. 4M show a sequence alignment of the modified sequence (1) (FIG. 3E), Delta spike protein (2) (FIG. 3F); Omicron BA1 spike protein (3) (FIG. 3D); and various engineered coronavirus spike proteins, FKS01-FKS12 (4)-(16) (FIG. 3F-FIG. 3R). Accordingly, the FrankenSpikes (FKS) described here provide diverse epitopes into one engineered coronavirus spike protein. For visualization, a phylogenic tree diagram is provided here as an example that shows the sequence similarity (the length of the line increases with larger differences) between thus far established engineered spike proteins, such as FKS01 to FKS12 in the context of thus far observed SARS-CoV-2 observed variants, as shown in FIG. 5.

### Nucleic Acid Encoding Coronavirus Spike Protein

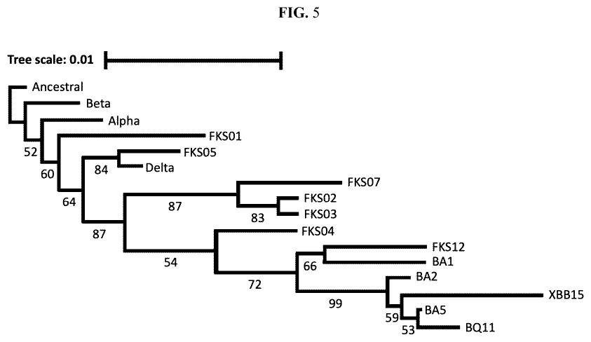

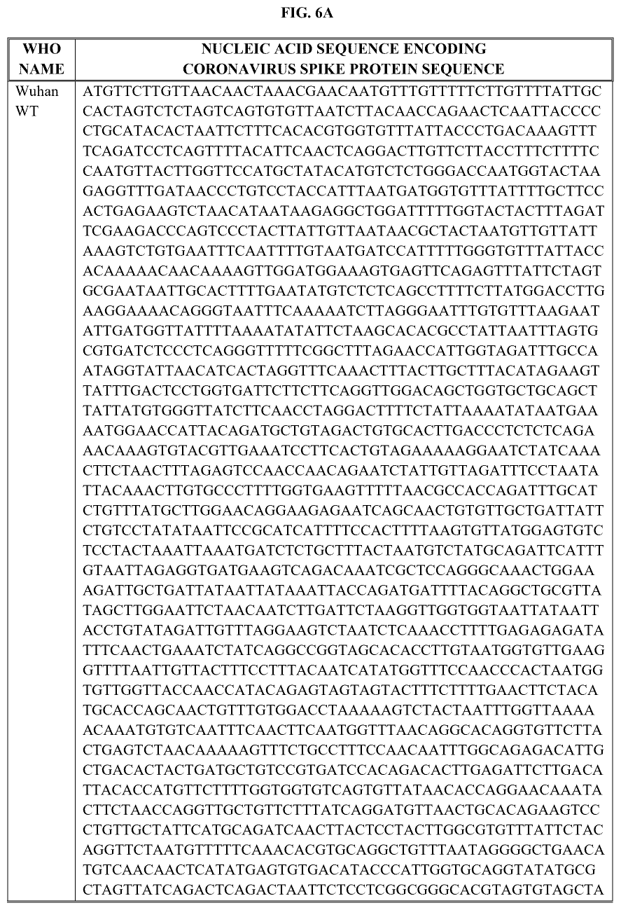

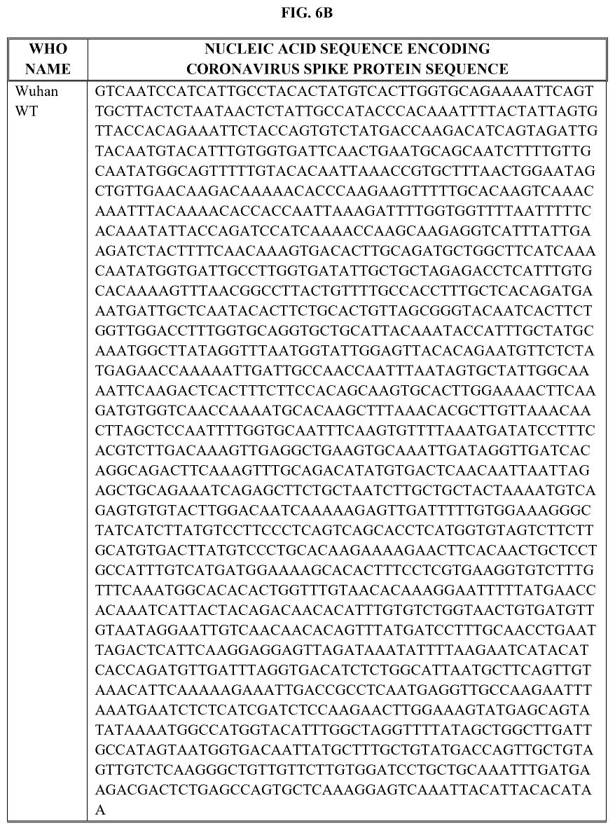

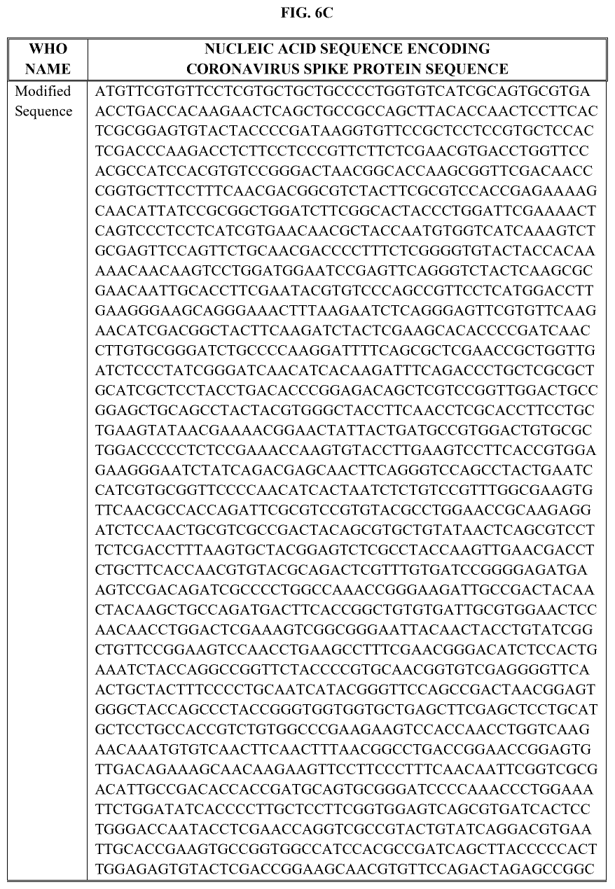

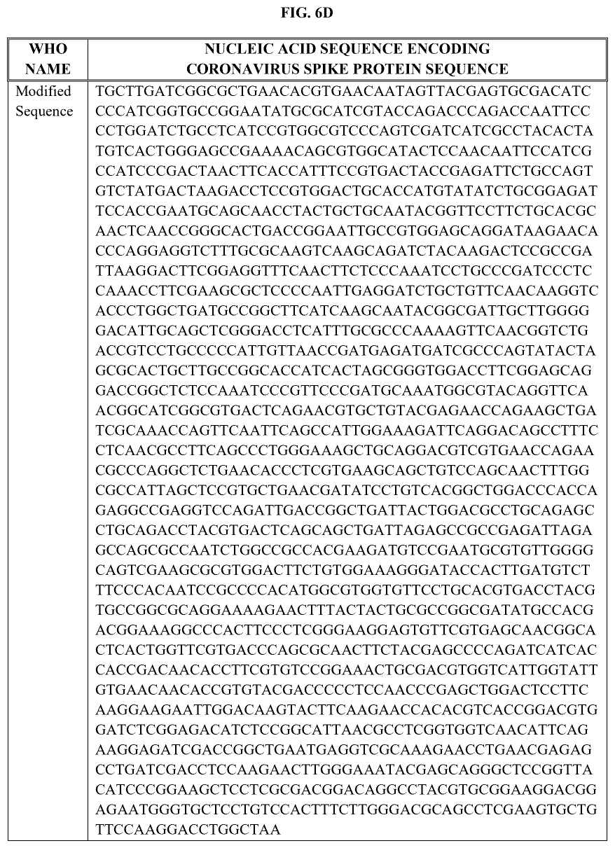

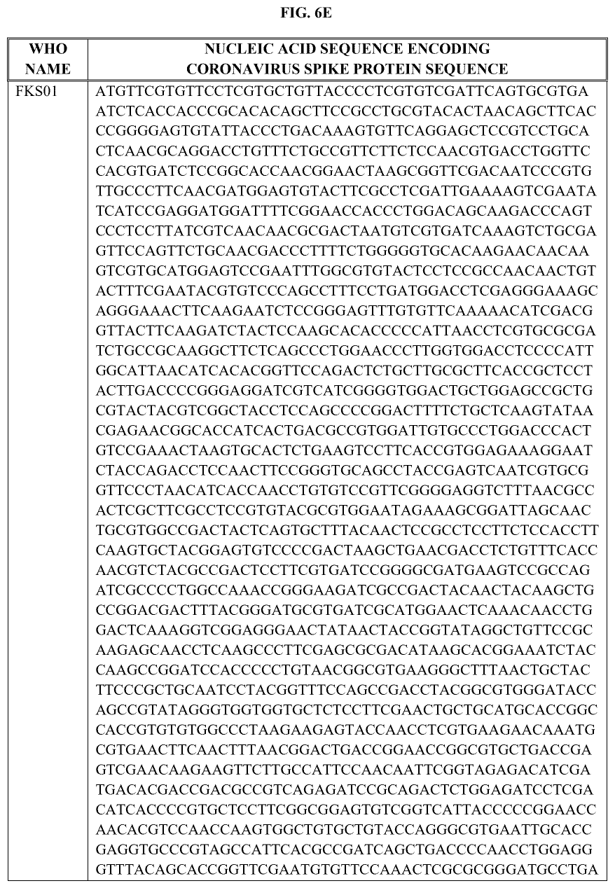

One embodiment provides for a nucleic acid molecule comprising a nucleic acid sequence encoding an engineered coronavirus spike protein as described here. The nucleic acid molecule can encode any of the modified coronavirus spike protein amino acid sequences described here. In some embodiments, the nucleic acid molecule encodes a natural coronavirus spike protein leader sequence, where an expression vector comprising this nucleic acid molecule can be introduced into a host cell in order to produce an engineered coronavirus spike protein vector using the methods described here. In one embodiment, the nucleotide sequence encoding the engineered coronavirus spike protein described here can be a nucleotide sequence of any desired species, such as for example, a human coronavirus spike protein, where the modified sequence is obtained by also optimizing the nucleotide sequence to be suitable for expression in host cells. Another embodiment can provide a nucleotide sequence encoding a coronavirus spike protein having a sequence of at least one of the amino acid sequences selected from the sequences of FIG. 2A-FIG. 2F, FIG. 3A-FIG. 3R, or FIG. 4A-FIG. 4M, or an amino acid sequence having identity of about 70% or greater, about 80% or greater, about 90% or greater, about 95% or greater, about 98% or greater, or about 99% or greater, compared to at least one of the amino acid sequences selected from the sequences of FIG. 2A-FIG. 2F, FIG. 3A-FIG. 3R, or FIG. 4A-FIG. 4M. Specifically, the nucleotide sequence can be selected from the sequences of FIG. 6A-FIG. 6F, for example, FIG. 6C-FIG. 6F, or a nucleotide sequence having a substantially similar sequence homology. A substantially similar sequence homology means that any nucleotide sequence that can have a nucleotide sequence identity of about 50% or greater, about 60% or greater, about 70% or greater, about 80% or greater, about 90% or greater, about 95% or greater, about 96% or greater, about 97% or greater, about 98% or greater, or about 99% or greater compared to by sequence alignment of at least a nucleotide sequence selected from FIG. 6A-FIG. 6F.

Some embodiments are directed to a nucleic acid molecule comprising a nucleotide sequence encoding an amino acid sequence of a protein comprising an engineered coronavirus spike protein described here, including at least one mutation identified in FIG. 2A-FIG. 2F and/or comprise the amino acid sequence identified in FIG. 3A-FIG. 3R.

Other embodiments can be directed to a nucleic acid molecule encoding an engineered coronavirus spike protein described here, where the nucleic acid molecule is a vector. Non-limiting examples of vectors useful for expressing any of the engineered coronavirus spike proteins described here include: expression vectors (e.g., pCG, pCC1-4K, pSTC, pUC57Kan, pXLG5, pXLG6) and viral vectors (e.g., adenovirus, AAV, lentivirus).

In some embodiments, the nucleic acid molecule comprises an mRNA encoding any of the engineered coronavirus spike proteins described here.

### Host Cell Expression System

For more than three decades, Chinese hamster ovary (CHO) cells have been used for large scale manufacturing of pharmaceutically relevant proteins, based on suspension cultivated cells in bioreactors. CHO cells are very diverse in their phenotypic potential due to their origin as immortalized cells that are constantly evolving and have been shown to be adaptable to very different modes for growth and production (Pino P. et al. Processes. 8(12):1539, 2020; Wurm F. M., Processes, 1:296-311, 2013; Wurm F. M., Nat Biotechnol. 22(11):1393-1398, 2004; Wurm F. M., and Wurm M. J., Processes, 5(2):20, 2017, all of which are incorporated herein in their entirety). In one embodiment, the mammalian cells that are useful in the methods of producing the described engineered coronavirus spike protein include a potent engineered-cell line for expression and scale-up in bioreactors derived from a non-engineered host cell line with selected phenotypes for manufacturing, such as for example, modified CHO cells (CHOExpress® cells; ExcelGene SA). This non-engineered host cell line has phenotypic features of exceptionally high growth rate, a high maximal cell density under batch and fed-batch culture with certain media formulations, and when transfected with suitable vectors, engineered progeny inherits these phenotypes with high fidelity. When using transfection appropriate expression vectors (e.g., vectors that drive the expression of the gene of interest (GOI) constitutive expression vectors) and appropriate selection and clonally-derived cell populations, the derived cell lines have a high synthetic capacity, and a high viability under fed-batch cultures, during which engineered product formation will occur. In another embodiment, the mammalian engineered cells are fast-growing, high yielding (>5 g/L with many protein targets), robust at high densities (>20 million cells/mL, up to 50 million cells/ml in Fed-batch processes), with a very high sub cultivation ratio (>1/30), ranging from subcultivation ratio of 1 to 2 to 1 to 100, and have the highest synthetic capacity, and the ability to maintain high viability (>90%) over an extended number of days, for example, 7 days, 11 days, 14 days, 17 days, greater than 7 days, greater than 11 days, greater than 14 days, etc. A further embodiment is directed to mammalian engineered cells that are modified Chinese hamster ovary (CHO) cells having these features, including, e.g., CHO-coronavirus spike protein, or such as but not limited to clonally derived cell populations such as, CHO-coronavirus spike protein_c112, CHO-coronavirus spike protein_c423, and the like. The engineered CHO cells described here can rapidly grow (under 20 hours/cell doubling) through the described culture program, and more specifically, for growth in animal component-free media or in chemically defined media. These engineered CHO cells have been generated from a non-engineered host cell line CHOExpress® cells—grown for three decades in animal-component free media that can be traced back to an initial CHO cell line obtained from an academic laboratory (Puck T T, et al. J Exp Med., 108(6):945-56, 1958).

In one embodiment, the compositions and formulations of the media, i.e., the production medium and the feed media used for culturing the mammalian host cells from which the engineered coronavirus spike protein is derived, are known by their name and concentration of each component, such that certain components of the media can be modified in concentration or can be removed entirely. Also, the addition of certain components can be done without negatively impacting the overall performance of the medium, but by enhancing the productivity and/or the quality of the desired coronavirus spike protein. These modifications can, accordingly, influence the secondary modifications of the coronavirus spike protein molecules produced by these cells during a fed-batch process.

### Expression Vector System for Transfection and Selection of Engineered Cell Populations

Another embodiment of the disclosure provides a nucleic acid construct comprising a nucleic acid molecule containing one or more nucleotide sequences encoding the desired engineered coronavirus spike protein, where the desired engineered coronavirus spike protein can include, for example, an engineered coronavirus spike protein. The construct can comprise an expression vector into which a sequence has been inserted, such as in a cassette. The expression vector can include the coding sequence for an engineered coronavirus spike protein, such as an engineered coronavirus spike protein. For example, an expression vector comprising a nucleic acid sequence encoding an engineered coronavirus spike protein and a selectable marker sequence, where both are positioned in opposite reading frames and in between a 5′ Inverted Terminal Repeat (5′ ITR) and a 3′ Inverted Terminal Repeat (3′ ITR) can be useful for transforming host cells in order to produce engineered coronavirus spike protein, where the selectable marker sequence comprises an antibiotic, e.g., puromycin, resistance gene sequence. A further embodiment can provide an expression vector comprising a nucleic acid molecule containing a nucleotide sequence encoding an engineered coronavirus spike protein polypeptide sequence having about 70% or greater identity to, about 75% or greater identity to, about 80% or greater identity to, about 85% or greater identity to, about 90% or greater identity to, about 95% or greater identity to at least one sequence of: FIG. 2A-FIG. 2F;

FIG. 3A-FIG. 3R; FIG. 4A-FIG. 4M. Yet another embodiment can provide for a nucleic acid molecule containing at least one nucleotide sequence encoding an engineered coronavirus spike protein polypeptide sequence, where the nucleotide sequence has about 70% or greater identity to, about 75% or greater identity to, about 80% or greater identity to, about 85% or greater identity to, about 90% or greater identity to, about 95% or greater identity to at least one sequence of: FIG. 6C-FIG. 6F.

In one embodiment, an expression vector can be used to transfer a nucleic acid sequence encoding the desired protein, for example, but not limited to an engineered coronavirus spike protein or an engineered coronavirus spike protein, into at least one host cell (e.g., in vitro). Expression vectors can be equipped with a nucleic acid sequence encoding a selectable marker, restriction enzyme sites, appropriate control elements, such as promoter and termination sequences, among other components. The expression vector can also comprise regulatory sequences, including, but not limited to, non-coding sequences, such as, for example, introns and control elements, i.e., promoter and terminator elements or 5′ and/or 3′ untranslated regions, that can be useful for expressing the coding sequence in host cells. Suitable vectors and promoters are known to those of ordinary skill in the art, many of which can be commercially available.

Non-limiting examples of suitable promoters can include constitutive promoters and inducible promoters, such as for example, a CMV promoter, an SV40 early promoter, an HSV promoter, an EF-1 a promoter, an actin promoter, and the like. Briefly, for expression purposes, a host cell can recognize a promoter sequence, where the promoter sequence is a DNA sequence. The promoter can be operably linked to a DNA sequence encoding a protein of interest, such as for example, an engineered coronavirus spike protein. The promoter can be positioned with respect to an initiation codon of the DNA sequence encoding the desired engineered coronavirus spike protein in the expression vector in a manner such that the promoter can drive transcription or translation of the nucleic acid sequence encoding the coronavirus spike protein. The promoter sequence can contain transcription and translation control sequences which mediate the expression of the engineered coronavirus spike protein.

An appropriate selective marker will generally depend on the host cell, and appropriate markers for different hosts are commonly known and used in the art. Such selectable markers can confer to transformants the ability to utilize a metabolite that is usually not metabolized by the host cell. A selectable marker can confer the ability of transformants to grow in the presence of an antibiotic, such as for example, puromycin, where the selectable marker is a puromycin resistant gene (Puro-r). The selectable marker coding sequence can be cloned into a suitable plasmid using methods generally employed in the art. Examples of suitable plasmids include pXLG5 and/or pXLG6. Conventional techniques of molecular biology, engineered DNA, immunology, and the like are within the skill of the art. After nucleic acid sequences that encode the engineered coronavirus spike protein, or other protein of interest, have been cloned into the construct or expression vector, the construct or expression vector can be used to transform at least one host cell in order to express an engineered coronavirus spike protein. The host cell that can be transformed for the purpose of expressing an engineered coronavirus spike protein according to the embodiments described here can be chosen from a wide variety of host cells. The various examples of expression vector components and host cells presented here are not meant to limit their scope but can be employed in practicing the aspects and embodiments presented here.

Another embodiment can provide an expression vector, comprising: a nucleic acid molecule containing a nucleotide sequence encoding a coronavirus spike protein, where the nucleic acid molecule is positioned in a multiple cloning site; an intron upstream of the nucleic acid molecule; a cytomegalovirus (CMV) promoter upstream of an intron; a 5′ Inverted Terminal Repeat (5′ ITR) upstream of the CMV promoter; a poly-adenosine tail signal sequence downstream of the nucleic acid molecule; a replication origin sequence downstream of the nucleic acid molecule; a selectable marker sequence downstream of the replication origin sequence; and a 3′ Inverted Terminal Repeat (3′ ITR) downstream of the selectable marker sequence. The selectable marker sequence can comprise in one embodiment, for example, a nucleic acid sequence of a puromycin resistance gene, where a person of skill in the art would understand how to select an appropriate selectable marker sequence and use its related counterpart, i.e., the antibiotic, such as puromycin, in order to select the clonally-derived cells containing the gene of interest, e.g., expressing engineered coronavirus spike protein, from the cell culture. Moreover, the person of skill in the art would also understand to position the nucleic acid molecule with the gene of interest and the selectable marker sequence in opposite reading frames and between the 5′ inverted terminal repeat (ITR) and the 3′ ITR.

Another embodiment of the expression vector can be directed to the nucleic acid molecule containing a cDNA sequence encoding an engineered coronavirus spike protein. Non-limiting selectable marker sequences can include an antibiotic resistance sequence, a thymidine kinase sensitive to ganciclovir selection, triclosan resistance sequence, a metabolic selection sequence, such as for example, a sequence comprising a dihydrofolate reductase gene or a glutamine synthetase gene, and the like, or combinations thereof. In one embodiment, the selectable marker sequence and/or antibiotic resistant gene is a puromycin resistance gene, an ampicillin resistance gene, a zeocin resistance gene, a geneticin resistance gene, a gene for any other desired selectable marker, such as for example, dihydrofolate reductase or glutamine synthetase, and the like, or combinations thereof. Another embodiment can be directed to an expression vector where the nucleic acid molecule and the selectable marker sequence are positioned in opposite reading frames and in between the 5′ ITR and the 3′ ITR. A further embodiment of the expression vector provides a nucleotide sequence encoding an engineered coronavirus spike protein polypeptide sequence (FIG. 3A-FIG. 3R) or encoding any other engineered coronavirus spike protein sequence (see, e.g., FIG. 2-FIG. 4), where the nucleotide sequence can be selected from, for example, FIG. 6C-FIG. 6F. In another embodiment, the expression vector comprises a nucleotide sequence having about 40% or greater identity to, about 50% or greater identity to, about 60% or greater identity to, about 70% or greater identity to, about 75% or greater identity to, about 80% or greater identity to, about 85% or greater identity to, about 90% or greater identity to, about 95% or greater identity, or about 99% or greater identity to at least one of the engineered coronavirus spike protein sequences of FIGS. 2-4, e.g., FIG. 2A-FIG. 2F, FIG. 3A-FIG. 3R, FIG. 4A-FIG. 4M, or the nucleotide sequence of FIG. 6C-FIG. 6F.

In yet a further embodiment, a co-transfection system comprising a donor vector expressing the gene of interest (GOI), i.e., encoding the engineered coronavirus spike protein described here or other engineered viral protein of interest, and a mobilizing vector expressing the gene of a transposase that recognizes Inverted Terminal Repeats (ITRs) framing the sequence of the GOI, can be used to efficiently incorporate the gene of interest, for example, the nucleic acid sequence of coronavirus spike protein or other engineered viral protein of interest into at least one host cell for producing the engineered coronavirus spike protein or other engineered viral protein of interest. As would be understood by a person of skill in the art, the method of co-transfecting a donor vector comprising any gene of interest as well as the other methods disclosed here can be utilized in order to produce an engineered protein encoded by the associated gene of interest. A further embodiment of the method of producing an engineered coronavirus spike protein or other engineered viral protein of interest can be directed to the step of introducing comprising co-transfecting the host cell with a vector containing the first nucleic acid sequence encoding an engineered coronavirus spike protein or other engineered viral protein of interest and with a vector containing the additional nucleic acid sequence encoding a transposase. An additional embodiment of the method can provide a helper vector or expression vector comprising a nucleic acid sequence or helper mRNA encoding a transposase that is introduced into the host cell or cell line with a vector or expression vector containing a nucleic acid sequence encoding an engineered coronavirus spike protein or other engineered viral protein of interest, where the nucleic acid sequence comprising the gene of interest (e.g., encoding: coronavirus spike protein, engineered coronavirus spike protein, other engineered viral protein of interest, etc.) integrates into the genome of the host cell and not the nucleic acid sequence from a mobilizing or helper vector (e.g., use of mRNA transposase in transfections without the use of any plasmid containing the DNA for transposase).

Briefly, a transposon is a genetic element that allows for efficient transposition between vectors and chromosomes by a “cut-and-paste” mechanism. Since the transposase expressed by the mobilizing or helper vector recognizes the transposon-specific ITRs of the donor vector containing the gene of interest, the transposase can “cut” the donor vector at the ITRs and then “paste” the donor vector sequence comprising the gene of interest and selectable marker sequence into the chromosomal DNA of the host cell, for example into TTAA chromosomal sites. Advantage of the transposon, for example, the piggyBac, system is that the sequence size for transposition is essentially unrestricted, it is non-viral, and highly efficient. Non-limiting examples of a transposase useful in embodiments of the disclosure include: piggyBac, Tol-2, Sleeping Beauty, Leap-In, and any other “cut-and-paste” transposases, or the like. Expression vectors or helper vectors comprising a transposase can include pD2500 vectors, particularly for the Leap-In transposase (ATUMSM; https://www.atum.bio/products/expression-vectors/mammalian#3; Newark, CA) or the vectors for the Tol-2- or Sleeping Beauty-based transfections (Balasubramanian S. Thesis No. 6563 (2015) “Study of Transposon-Mediated Cell Pool and Cell Line Generation in CHO Cells,” Swiss Federal Institute of Technology (EPFL), Lausanne, Switzerland).

In yet a further embodiment, the expression vector that is introduced into cells and its subsequent expression of the GOI can contain additional sequences, such as CHO-cell derived endogenous retroviral sequences, that can facilitate the integration of such expression vectors into the active chromatin of the non-engineered CHOExpress® host cell line. Such endogenous retroviral sequences can belong to the family of A-type retroviral sequences (Anderson K, et al. Virology 64, 5, 2021-2032, 1990), the family of C-type sequences (Anderson K, et al. Dev. Biol. Stand. 75:123-132, 1991), or any other family of repetitive sequences in eukaryotic genomes that are clustered preferentially in active regions of the genome (Mager D L and Stoye J P. 2014. Microbiol. Spectr. 3(1): MDNA3-0009-2014). These additional endogenous retroviral DNA sequences can either represent a full-length (non-functional) retroviral sequence or shorter fragments thereof. This approach can mediate homologous recombination events in cells that result in integration of the GOI sequences into a region of the genome that contains an endogenous retroviral DNA sequence (Wurm F M, et al. “Retrotargeting: Use of defective retroviral DNA fragments to improve engineered protein production in mammalian cells.” Animal Cell Technology: Products for Today, Prospects for Tomorrow, edited by R. E. Spier et al., Butterworth-Heinemann Ltd., 1994, pp. 24-29).

In yet another embodiment, the expression vector for the GOI interest can be constructed for high-level productivity from engineered cells by combining a transposon-based gene transfer approach with a homologous recombination approach for integration into active chromatin of the genome of the DNA receiving cells. Non-limiting approaches for transfecting cells and selecting engineered cell populations include those that use targeted gene transfers into cells via zinc (Zn)-finger nucleases (Bibikova M, et al. Science. 300(5620):764, 2003), single chain homing nucleases (Grizot S, et al. Nucleic Acids Research. 37(16):5405-5419, 2009), or CRISPR/Cas 9 processes (Jinek M, et al. Science 337(6096):816-821, 2012).

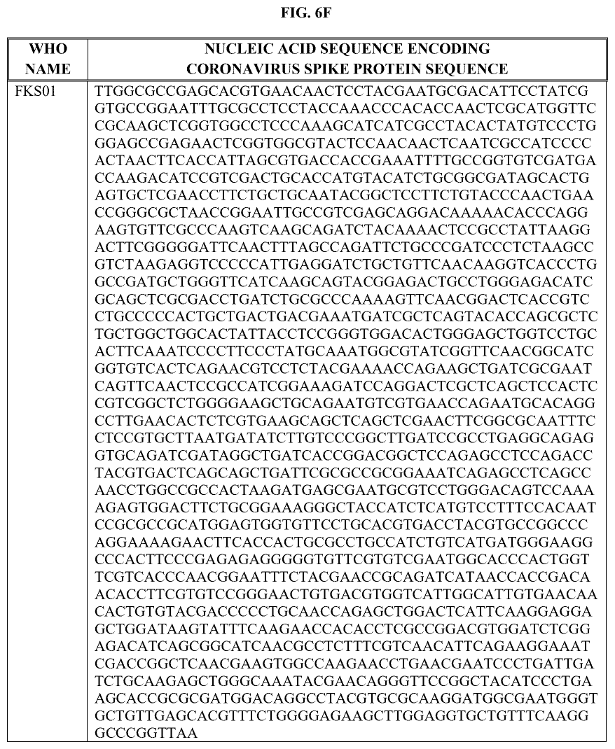

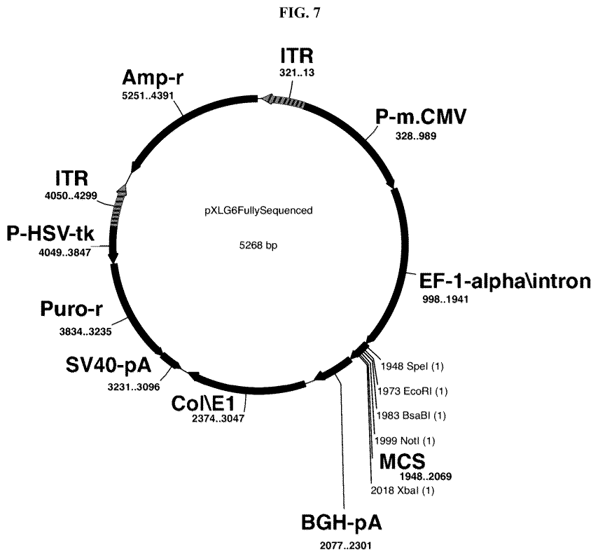

Some embodiments of the disclosure are directed to expression vector constructs used to obtain high-level engineered coronavirus spike protein expression from transfected mammalian cells. The plasmid vector pXLG6-coronavirus spike protein comprises a nucleic acid sequence that encodes the complete engineered coronavirus spike protein sequence described here, including the corresponding leader sequence, which is inserted in the multi-cloning site (MCS) of the pXLG6 plasmid vector. See, FIG. 7. Also encompassed in an embodiment of the disclosure is a mobilizing or helper vector, i.e., the plasmid pXLG 5 comprising a transposase, such as but not limited to, a PiggyBac transposase (mPBase), or other transposases including, but not limited to Tol-2, Sleeping Beauty, Leap-In, any other “cut-and-paste” transposase, and optimized versions thereof. See, FIG. 8. The pXLG 5 is co-transfected with pXLG6-coronavirus spike protein in mammalian host cells, such as for example, modified CHO cells (e.g., CHOExpress® cells; ExcellGene S.A.) or the like, where the modification allows for cells to grow to a high cell density, for example, but not limited to more than 20 million cells/mL, a high sub-cultivation ratio of, such as but not limited to more than 1/20, and engineered CHO cell process yields having expression levels of about 500 mg/L or greater (e.g., 600 mg/L, 700 mg/L, 800 mg/L, 900 mg/L, 1 g/L, 3 g/L, 5 g/L, 6 g/L, 7 g/L) among other advantages. The co-transfection into mammalian host cells can occur in varying amounts where the transposase expression vector generally has a low molar ratio relative to the GOI (e.g., the engineered coronavirus spike protein expressing vector described here), as the transposase expression vector is a mobilizing or helper vector which aids in the integration or incorporation of the GOI into the genome of the host cells, while the transposase nucleic acid sequence avoids integration. Non-limiting weight/weight ratios of the transposase vector to the GOI vector can include, but are not limited to, about 1:1, about 1:3, about 1:9, about 1:10, about 0.75:9.25, about 0.5:9.5, about 0.25:9.75, about 0.1:9.9, less than about 1:10, less than about 1:9, less than about 1:3, less than about 0.75:9.25, less than about 0.5:9.5, less than about 0.25:9.75, less than about 0.1:9.9, greater than about 0.1:9.9, greater than about 0.25:9.75, greater than about 0.5:9.5, greater than about 0.75:9.25, greater than about 0.9:9.1, greater than about 1:9, greater than about 1:10, about 1:10 to about 0.1:9.9, about 0.75:9.25 to about 0.25:9.75, about 0.5:9.5 as well as any intervening or additional ratios that allow for successful incorporation of the GOI into the host cell genome. Since the transposase vector does not contain ITRs, the transposase would not integrate into the host cell genome, would however over time be eliminated from cells by degradative intracellular processes and thus, the transposase vector is merely active in this co-transfection protocol for a short time (transiently) to assist with the incorporation or integration of the GOI (e.g., coronavirus spike protein expression cassette) into the host cell genome. The same principle applies when Transposase-encoding mRNA is used in a co-transfection with GOI-encoding expression vector DNA.

In one embodiment, the pXLG 6 expression vector cassette for the gene of interest (GOI; e.g., encoding an engineered coronavirus spike protein described here) can contain a strong constitutive promoter/enhancer derived from a mouse Cytomegalovirus (mCMV) sequence and other useful elements, such as splice-donor sequences, and another expression cassette for the constitutive expression of a selective marker (e.g., the gene encoding for puromycin resistance (Puro-r): PAC—puromycin N-acetyl-transferase) driven by a Herpes Simplex Thymidine Kinase promoter (HSV TK). The GOI sequence, a nucleic acid sequence encoding an engineered coronavirus spike protein sequence of the disclosure (see, e.g., FIGS. 2-4), can be cloned into the multi-cloning site (MCS) of pXLG 6 (see, FIG. 7).

These two expression cassettes can be framed by inverted terminal repeat sequences, 5′ ITR and 3′ ITR. These two ITRs or other ITRs are recognized by a transposase protein, including those, but not limited to, the PiggyBac transposon of the cabbage looper moth (Trichoplusia ni), the Tol-2 transposon (Kawakami K. Genome Biol. 8(Suppl 1):S7, 2007) or the Sleeping Beauty transposon (Aronovich E L, et al. Human Molecular Genetics, 20:R14-R20, 2011), the Leap-In transposase (ATUMSM; https://www.atum.bio/products/expression-vectors/mammalian #3; Newark, CA), or any other DNA mobilizing system using transposases. In order to introduce the transposase protein to host cells, a second vector that expresses, e.g., a PiggyBac transposase or a Sleeping Beauty transposase, can be co-transfected with the plasmid vector comprising the gene of interest. One embodiment relates to the second vector, pXLG 5, comprising a PiggyBac transposase. See, FIG. 8. The pXLG 5 plasmid vector contains a corresponding expression cassette encoding the PiggyBac transposase (mPBase). In an embodiment of the disclosure, the ratio of plasmid DNA in co-transfections of CHO cells of GOI vector (e.g., pXLG6-coronavirus spike protein vector) and transposase vector (pXLG5-mPBase vector or helper vector) is 9:1 (by weight). Thus, 90% of the transfection cocktail contains the GOI vector. Higher and lower ratios of helper vector to GOI vector are not excluded and can include ratios, such as but not limited to weight percentages of about 1% of helper vector to about 99% of GOI vector, 5% helper vector to about 95% of GOI vector, about 15% helper vector to about 85% GOI vector, 20% of helper vector to 80% of GOI vector, and the like, such that there is a successful co-transfection that results in the production of active, mature engineered coronavirus spike protein.

Upon transfection with a chemical transfection reagent (CHO4Tx® kit; ExcellGene SA) and following an optimized procedure, hundreds, if not thousands of plasmids—here a mixture of two different nucleic acid vectors—can be transferred into the nucleus of the host cells, e.g., CHO cells. The transposase vector can drive the transcription of the transposase gene and the synthesis of the transposase. The transposase proteins can then recognize the ITR sequences in the GOI vector and excise them from the plasmid. Subsequently, the excised GOI cassette can be integrated into the genome of the host cell mediated by the transposase (Matasci M, et al. Biotechnol Bioeng. 108(9):2141-50, 2011 Apr. 25 Epub). Another embodiment is directed to modified host CHO cells with at least one GOI cassette encoding an engineered coronavirus spike protein. In a further embodiment, about 5-30 copies, about 5-15 copies, or 10-20 copies, of the GOI cassettes can be integrated into the genome of the cloned engineered CHO cell lines derived from such transfections. Since the PiggyBac transposase has a preference for integration into active chromatin, the expression levels of such engineered cell lines are found to be high.

### Transfection and Selection

Another embodiment of the disclosure can be directed to the transfection and selection of engineered coronavirus spike protein expressing cells. Antibiotic resistance selection of the modified CHO cells co-transfected with the donor plasmid vector comprising the gene for coronavirus spike protein and the mobilizing plasmid vector comprising a transposase gene, such as but not limited to the PiggyBac transposase gene, suggests that the surviving cells have both the GOI (e.g., encoding engineered coronavirus spike protein) and the antibiotic resistance gene, such as for example, a puromycin resistance gene integrated and expressed, or any other resistance providing DNA that is in the plasmid vector and has transformed the host cells, while the transposase gene of the mobilizing vector is not transformed in the host cells. Both the transfection and the selection can occur with cells that grow rapidly without any aggregation under suspension culture in cell culture media and are not at any time exposed to animal component-derived substances. The selection pressure, during about 7 days to about 10 days after transfection, can be maintained under very stringent conditions. These conditions include replacing the medium containing the selection-providing agent every day. Once the cells rapidly grow again and cell viability has been re-established to high values, the cultures can be sub-cultivated using typical and commonly used techniques. This heterogenous population of cells, once further grown and expanded in the absence of any antibiotic selective agent, is considered therefore engineered and expresses the engineered coronavirus spike protein at very high levels, such as but not limited to about 500 mg/L or greater (e.g., 600 mg/L, 700 mg/L, 800 mg/L, 900 mg/L, 1 g/L, about 2 g/L, about 3 g/L, about 4 g/L, about 5 g/L, about 6 g/L, about 7 g/L, about 8 g/L, about 9 g/L, about 10 g/L; 500 mg/L-10 g/L; 10 g/L or less), or any intervening amounts. Thus, an embodiment of the disclosure is directed to engineered cells expressing high levels of active, engineered coronavirus spike protein and clonal cell lines thereof.

In one embodiment, the modified CHO cells can be transfected with a high efficiency expression vector (e.g., by transposase-mediated gene integration or other known methodology) containing the engineered coronavirus spike protein gene. Engineered pools expressing coronavirus spike protein can be isolated and subsequently, clonal-derived cell lines can be selected by single-cell cloning and expansion. Briefly, modified CHO cells can be co-transfected with the donor GOI or engineered coronavirus spike protein expression vector and the mobilizing or helper transposase vector at a ratio where there is more donor GOI expression vector to transposase vector or less transposase vector to donor GOI expression vector. The donor coronavirus spike protein vector to helper transposase vector ratio can include, but is not limited to, 9:1 (w/w), 9.25:0.75 (w/w), 9.5:0.5 (w/w), 9.75:0.25 (w/w), 9.9:0.1 (w/w), and 10:1 (w/w), or intervening ratios. For example, the ratio of pXLG-6 coronavirus spike protein vector to pXLG-5 transposase vector can include, but is not limited to, 9:1 (w/w), 9.25:0.75 (w/w), 9.5:0.5 (w/w), 9.75:0.25 (w/w), and 9.9:0.1 (w/w). The transfected cells can be maintained under suspension culture, where the medium can be supplemented with a selective agent, such as for example, puromycin at 50 μg/ml and changed daily with puromycin-supplemented medium for about 7 days to about 10 days or until a healthy population of cells has been recovered showing a cell viability of greater than or about 50%, greater than or about 60%, greater than or about 70%, greater than or about 80%, greater than or about 90%, greater than or about 95%, or about 100%. Where the cells have reached a viability of at least about 90%, the cells can be further sub-cultivated in a medium without puromycin since the selection has already occurred. A typical sub-cultivation schedule of about 3 days to about 4 days can continue. The sub-cultivated cells can be tested for successful engineered protein expression and can be cloned using a limited dilution approach, a single-cell printer (Cytena AG, Freiburg Germany), or both techniques. Up to about 1,000 clonally-derived cell populations can be expanded and investigated for optimized protein production for coronavirus spike protein.

One embodiment can be directed to a method for producing an engineered coronavirus spike protein as described here, comprising: a) introducing a host cell with an expression vector comprising a nucleic acid molecule containing a nucleic acid sequence which encodes an engineered coronavirus spike protein to isolate a transformant, i.e. an engineered cell, expressing the engineered coronavirus spike protein; b) culturing the host cells with the transformant, engineered cells, or expression vector comprising the nucleic acid molecule that comprises a nucleic acid sequence which encodes a coronavirus spike protein under conditions which allow for expression of an engineered coronavirus spike protein; and c) isolating the engineered coronavirus spike protein from the engineered cells, thereby producing the engineered coronavirus spike protein. Another embodiment of the method described herein can provide for the nucleic acid molecule comprising a nucleic acid sequence encoding an engineered coronavirus spike protein that is a CHO-cell codon-optimized sequence.

A further embodiment can provide for the introducing step of the method described here comprising co-transfecting the engineered coronavirus spike protein expression vector or expression vector comprising coronavirus spike protein variants and an expression vector encoding a transposase, where the transposase expression vector is a helper or mobilizing vector which assists with the incorporation of the gene of interest into at least one host cell genome. Co-transfecting transposase and a vector containing a gene of interest can result in the delivery of the plasmid-excised coronavirus spike protein expression cassette into the genome of the non-engineered host cells. Another embodiment can be directed to a transposase that is, for example, a piggyBac transposase and a mobilizing vector, helper vector, or an expression vector encoding, for example, a piggyBac transposase, where the transposase gene is introduced into the host cell or host cell culture and not introduced into the genome of the host cells. The expression vector encoding a transposase is a “helper vector” for the incorporation of the gene of interest, i.e., in one embodiment, the engineered coronavirus spike protein expression cassette, into the host cell genome. Another embodiment can be the use of an in vitro synthetized mRNA preparation that encodes for a transposase, instead of using an expression vector for transposase.

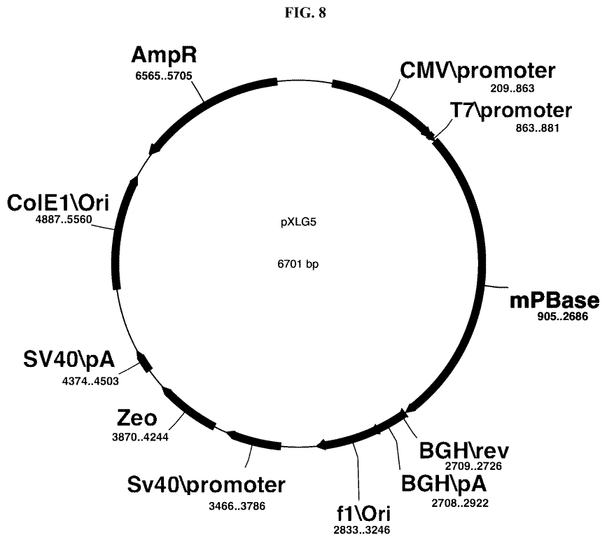

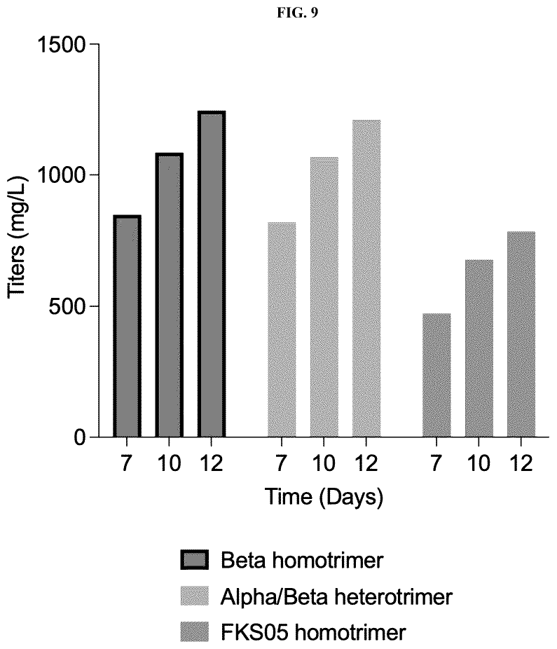

In some embodiments, more than one (e.g., 2, 3, 4, 5, 6) expression vector containing a nucleic acid sequence encoding any coronavirus spike protein variant (e.g., Wuhan (Ancestral), Alpha, Beta, Gamma, Delta, Epsilon, Eta, Iota, Kappa, Lambda, Mu, Zeta, B.1.617.3, Omicron (B.1.1.529, BA.1, BA.1.1, BA.2, BA.3, BA.4, BA.5); FIG. 3A-FIG. 3D) with stability and/or manufacturability modifications described here (i.e., a non-functional protease cleavage site (e.g., a non-functional furin cleavage site at amino acid positions 682-685 (GSAS (SEQ ID NO: 28)) relative to the modified amino acid sequence of FIG. 3E); prolines at amino acid positions 817, 892, 899, 942, 986, and 987 (i.e., P817, P892, P899, P942, P986, and P987) relative to the modified amino acid sequence of FIG. 3E; replacement of a transmembrane domain and an intracellular tail with T4 foldon motif also known as a T4 fibritin trimerization foldon motif (e.g., GYIPEAPRDGQAYVRKDGEWVLLSTFL (SEQ ID NO: 3) at amino acid positions 1211-1237 relative to the modified amino acid sequence of FIG. 3E)) can be co-transfected such that a combination of monomers can be assembled in the endoplasmic reticulum (ER) as hetero-trimeric and/or homo-trimeric spike proteins or MonsterSpikes. Without being bound by theory, assembly of two or three different monomer spike proteins into one molecule can produce a broad range of antibodies. For example, two different expression vectors for the proteins of interest can be co-transfected into CHO cells as described here to express Alpha variant spike protein (A), also referred to as CVD6, and Beta variant spike protein (B) also referred to as CVD7, which are purified using encoded Histidine (His)-tag sequences. Instead of generating only homo-trimers, this method can also generate hetero-trimeric structures since these molecules self-assemble, producing a secreted population of molecules which can include a mixture of AAA, BBB, AAB, and ABB. Depending on the ratio of molecules transfected, the generation of the heterotrimeric molecules can be favored over those that are homotrimeric. Another example can be directed to co-transfecting three different vectors encoding three different spike protein variants (e.g., AAA, BBB, CCC, AAB, AAC, ABB, ACC, ABC, BBC, CBB, CCB). Depending on the ratio of molecules transfected, generation of the heterotrimeric molecules can be favored over homotrimeric molecules, and also favor heterotrimeric molecules that have three, instead of two different spike molecules integrated into single trimeric structures. Protein preparations of these hetero-trimeric structures can present for the cases of AAB, ABB, ACC, ABC, BBC, CBB and CCB, two or occasionally three different antigenic surfaces for the trimer. These preparations are defined here as “MonsterSpikes” if composed of spike monomers of past or present virus variants of concern. FIG. 9 shows the results of cell culture production comparing a homo-trimeric coronavirus spike protein (Beta homotrimer) to a hybrid or hetero-trimeric coronavirus spike protein of Alpha and Beta variant spike protein (Alpha/Beta heterotrimer) and one FrankenSpike homotrimer (FKS05). Pooled populations of Beta homotrimer and Alpha/Beta heterotrimer were found to have similar titers over days 7, 10, and 14 demonstrating that heterotrimers could be produced at the same rate as homotrimers. In some embodiments, a trimeric protein can comprise more than one engineered coronavirus monomer of the spike protein. Some aspects are directed to a preparation comprising an engineered coronavirus spike protein comprising a virus variant-spike monomer, where when, for example, in a hetero-trimeric structure, there is a preferential presence of one particular spike molecule derived from one particular SARS-CoV2 virus variant or one particular FrankenSpike. This can be accomplished by modifying the ratio of transfections from, for example, 1:1:1 (for three different spike molecules) to a 3:1:1 ratio that favors a particularly desired variant, for example, Omicron in Omicron:FKS2:FKS5, where the desired variant is in a greater amount than the other variants in the ratio. In some embodiments, the trimeric protein comprises more than one engineered coronavirus monomer of the spike protein, where a preparation of engineered coronavirus spike proteins comprises a virus variant spike monomer capable of forming a hetero-trimeric structure with a specific molecule derived from a specific SARS-CoV2 virus variant or a specific FrankenSpike. FIG. 9 demonstrates that co-transfection using two different spike protein variant vectors (Alpha and Beta, each containing His-Tag sequences for purification purposes) results in the production of hybrid spike protein variants having the same or similar titers as the Beta spike protein variant expressed by its vector alone.

Other embodiments can be directed to co-transfection of more than one (e.g., 2, 3, 4, 5, 6) expression vectors comprising nucleic acid sequences encoding the FrankenSpikes described here (e.g., FKS01-FKS13; FIG. 3F-FIG. 3R), which contain not only mutations for stability and manufacturability, but also additional mutations that present antigenic surfaces from different variants of concern. Such heterotrimeric spike preparations are referred to as Monster-FrankenSpikes. In additional embodiments, provided here are compositions or vaccines that produce the engineered coronavirus spike proteins described here in a mixture of molecules that simultaneously present different antigen structures, such as those found in the FrankenSpikes or MonsterSpikes of the disclosure.

Another embodiment can be directed to a host cell or host cell population that is a eukaryotic cell or a eukaryotic cell population. A further embodiment can provide for a non-engineered host cell population that is a Chinese hamster ovary (CHO) cell line. Yet another embodiment can be directed to a CHO cell line, where the CHO cell is an engineered CHO cell line. The CHO cell or CHO cell line can be a CHO cell line that has been modified to produce rapidly-growing hearty or robust cells in a high density which produces high yields of expressed protein. These modified CHO cells can easily scale up from small scale production to large scale manufacturing as well.

In a further embodiment, the non-engineered and engineered CHO cell line can be modified to rapidly grow in a culture medium essentially without some or any animal components (i.e., including human components or non-human animal components) or essentially without some or any immune response-inducing human components or non-human animal components. Another embodiment provides for methods disclosed here that are directed to a culturing step of the engineered CHO cells which occurs essentially without some or any animal (i.e., human or non-human) components. The culturing step occurs in a culture medium, where the culture medium contains less than about 5% (vol/vol), less than about 4% (vol/vol), less than about 3% (vol/vol), less than about 2% (vol/vol), less than about 1% (vol/vol) of any animal-derived contaminants, or no or essentially no contaminants, where contaminants can include animal-derived components, such as for example, human- and/or non-human-derived components, or any animal-derived components that can trigger an immune response. The culture medium can contain any additives which assist in the growth and expansion of the transformed host cells, including but not limited to feeds, amino acids, and insulin (e.g., human engineered animal origin free insulin) in an amount that facilitates to expression of engineered protein isolated from the host cells, such as for example, engineered coronavirus spike protein.

In yet another embodiment, the method described here can be directed to a culturing step that produces or yields about 500 mg/L or greater, 600 mg/L or greater, 700 mg/L or greater, 800 mg/L or greater, 900 mg/L or greater, 1 g/L or greater, about 2 g/L or greater, about 3 g/L or greater, about 4 g/L or greater, about 5 g/L or greater, about 6 g/L or greater, about 7 g/L or greater, about 8 g/L or greater, or about 10 g/L or greater, or about 15 g/L or greater of engineered coronavirus spike protein described here (e.g., SARS-CoV-2 spike protein), about 1 g/L to about 15 g/L of engineered coronavirus spike protein, about 2 g/L to about 6 g/L of engineered coronavirus spike protein, or about 3 g/L to about 15 g/L of the engineered coronavirus spike protein. Another embodiment can be directed to the culturing step that produces about 4 g/L to about 10 g/L of the engineered coronavirus spike protein.

A further embodiment provides for the method of producing an engineered coronavirus spike protein, wherein the culturing step comprises: selecting the host cell with the nucleic acid molecule expressing the engineered coronavirus spike protein described here, wherein the selected cells are clonally-derived cells expressing engineered coronavirus spike protein. The selecting step comprises: a) growing or culturing the clonally-derived engineered cells expressing engineered coronavirus spike protein in a culture medium; b) feeding the clonally-derived cells expressing engineered coronavirus spike protein with at least one feed; c) maintaining the culture medium at a cell culture temperature sufficient to maintain or promote normal, healthy cells; d) modifying or decreasing the cell culture temperature; e) growing or culturing the clonally-derived cells at the decreased cell culture temperature until the cells express the engineered coronavirus spike protein, for example, engineered coronavirus spike protein at a titer of about 500 mg/L or greater, 600 mg/L or greater, 700 mg/L or greater, 800 mg/L or greater, 900 mg/L or greater, 1 g/L or greater, about 2 g/L or greater, about 3 g/L or greater, about 4 g/L or greater, about 5 g/L or greater, about 6 g/L or greater, about 7 g/L or greater, about 8 g/L or greater, about 9 g/L or greater, about 10 g/L or greater, about 11 g/L or greater, about 12 g/L or greater, about 13 g/L or greater, about 14 g/L or greater, or about 15 g/L or greater, or to a point such that the clonally-derived cells are grown sufficiently to express engineered coronavirus spike protein at a desired titer of about 1 g/L or greater. Another embodiment can be directed to cells that express engineered coronavirus spike protein at a titer of about or greater than about 2 g/L, about or greater than about 3 g/L, about or greater than about 4 g/L, or about or greater than about 6 g/L, and in another embodiment, achieving these titers by or at Day 3, Day 5, Day 7, Day 10, Day 14, or Day 17 of cell culturing. A further embodiment can be directed to the host cells that express engineered coronavirus spike protein at a titer of about or greater than about 6 g/L at Day 17 of cell culturing. In yet a further embodiment, the cell culture temperature during the production phase, i.e. the culture of cells in bioreactors that terminates with the harvests of cells and culture medium, ranges from about 35° C. to about 38° C., or for example, about 37° C., or at the first days of culturing or Day 0 to Day 3 or Day 0 to Day 5, or starting at Day 0 of cell culturing or used interchangeably throughout the description, where Day 0 is the first day of culturing for production, or another appropriate day or range of days that is sufficient for achieving the desired protein levels encoded by the gene of interest, including for example, engineered coronavirus spike protein. One embodiment can be directed to a shifted, modified, or decreased cell culture temperature ranging from about 25° C. to about 34° C. or for example, about 31° C. to about 33° C., about 31° C., or about 33° C. by, at, or starting at Day 3 or Day 5 of cell culturing, or another appropriate day or range of days that is sufficient for achieving the desired protein levels encoded by the gene of interest, including for example, engineered coronavirus spike protein. A further embodiment can be directed to a decreased cell culture temperature ranging from about 31° C. to about 33° C. by, at, or starting at Day 3 or by, at, or starting at Day 5 of cell culturing, or another appropriate day or range of days that is sufficient for achieving the desired protein levels of, for example, engineered coronavirus spike protein (e.g., engineered SARS-CoV-2 spike protein), encoded by the gene of interest.

In another embodiment, the feeding step of the methods described here provides for at least one feed selected from a neutral feed, an alkaline feed, or another feed that is sufficient to maintain or promote normal healthy cells for achieving the desired protein levels expressed by the gene of interest, including for example, engineered SARS-CoV-2 spike protein. Yet a further embodiment provides a neutral feed having a concentration ranging from about 1% to about 10%, about 1% to about 8%, about 1% to about 6%, about 1% to about 5% of the total cell culture volume. Another embodiment can provide for the at least one feed comprising an alkaline feed. In a further embodiment, the alkaline feed can have a concentration ranging from about 0.1% to about 1%, 0.1% to about 0.8%, about 0.1% to about 0.6%, about 0.1% to about 0.5% of the total cell culture volume. One embodiment provides for at least one feed comprising a neutral feed and an alkaline feed. A further embodiment provides for a feed comprising a neutral feed and an alkaline feed in an amount about one-fifteenth (1/15), one-tenth (1/10), about one-eighth (⅛), about one-sixth (1/6), about one-fifth (⅕) of that of the neutral feed in a total cell culture volume. In one embodiment, the feeding step occurs every day or every other day, or any other feeding schedule which maintains or promotes normal, healthy cells for achieving the desired protein levels expressed by the gene of interest, including for example, engineered SARS-CoV-2 spike protein. In another embodiment, the feeding occurs continuously using controlled flow-rates for the neutral feed and/or for the alkaline feed.

Another embodiment can be directed to the methods of producing an engineered coronavirus spike protein where the culturing step during the production phase comprises an osmolarity of the cell culture of about 200 mOsm/kg to about 600 mOsm/kg, about 250 mOsm/kg to about 400 mOsm/kg, about 260 mOsm/kg to about 320 mOsm/kg, about 450 mOsm/kg to about 600 mOsm/kg, about 500 mOsm/kg or greater, about 550 mOsm/kg or greater, or any appropriate osmolarity which maintains or promotes normal, healthy cells for achieving the desired protein levels including, for example, engineered coronavirus spike protein, expressed by the gene of interest. In one embodiment, the osmolarity of the cell culture can be about 550 mOsm/kg or greater by or at Day 5 or later, or any another appropriate day or range of days that is sufficient for achieving the desired levels of protein encoded by the gene of interest, including for example, engineered SARS-CoV-2 spike protein.

A further embodiment provides a method for producing an engineered coronavirus spike protein, e.g., engineered SARS-CoV-2 spike protein, comprising: a) introducing into a host cell or a host cell population, e.g., eukaryotic, a first nucleic acid sequence encoding an engineered coronavirus spike protein (e.g., engineered SARS-CoV-2 spike protein) and at least an additional nucleic acid sequence encoding a transposase; b) culturing the host cell or host cell population under conditions which allow expression of the first nucleic acid sequence encoding an engineered coronavirus spike protein (e.g., engineered SARS-CoV-2 spike protein), where the additional nucleic acid sequence encoding, for example, a transposase, such as but not limited to for example, piggyBac, can also be in the cell culture and expressed in order to assist in the incorporation of the gene of interest encoding, for example, an engineered SARS-CoV-2 spike protein, where the host cell is transformed with a nucleic acid sequence encoding an engineered SARS-CoV-2 spike protein; c) selecting the host cell with the nucleic acid molecule expressing an engineered SARS-CoV-2 spike protein, wherein the selected cells are clonally-derived cells expressing engineered SARS-CoV-2 spike protein; and d) isolating the engineered SARS-CoV-2 spike protein from the host cell or eukaryotic host cell, thereby producing the engineered SARS-CoV-2 spike protein, where the step of isolating can comprise purifying the engineered SARS-CoV-2 spike protein.

In some embodiments, the GOI encoding any of the engineered coronavirus spike proteins described here can have additional modifications to enhance purification, for example, encoding affinity tags (e.g., His-tag, GST-tag). Additional embodiments can accomplish purification without the use of affinity tags and instead using affinity resins, such as those provided by REPLIGEN (NGL COVID-19 Spike Protein Affinity Resin) which purifies SARS-CoV-2 spike protein receptor binding domain (RBD) variants to high purity in a single chromatography step.

Another embodiment can provide for a eukaryotic host cell or a eukaryotic cell population transformed with a nucleic acid sequence encoding an engineered coronavirus spike protein. In another embodiment, the step of introducing can comprise co-transfecting the host cell with a vector containing the first nucleic acid sequence encoding a coronavirus spike protein and with a vector containing the additional nucleic acid sequence encoding a transposase, such as but not limited to, a piggyBac, Tol-2, Sleeping Beauty, Leap-In, and any other “cut-and-paste” transposase, or the like. Yet a further embodiment of the isolating step can provide a step of purifying the desired engineered coronavirus spike protein. The purifying step can be performed by at least one of, but is not limited to, any one or more of the techniques of: affinity chromatography including, for example, antibody- or ligand-based affinity chromatography, size exclusion chromatography, ion exchange chromatography, hydrophobic interaction chromatography, reverse phase chromatography, gel filtration, magnetic bead separation, selective precipitation, molecular weight-based membrane filtration or exclusion, buffer exchange, virus filtration, pH-based inactivation of viruses, and the like.

A further embodiment of the disclosure can be directed to the method of producing an engineered coronavirus spike protein, where the method comprises a culturing step in a culture medium that is essentially free of animal-derived components (i.e., human or non-human animal components) or proteins that induce an immune response, such as for example, human immunoglobulins, human serum albumin, non-human animal proteins, non-human animal immunoglobulins, non-human serum albumin, or any other contaminant. In a further embodiment, the method of producing an engineered coronavirus spike protein, where the method comprises a culturing step in a culture medium that contains less than about 5% (vol/vol) of animal-derived components, less than about 4% (vol/vol) of animal-derived components, less than about 3% (vol/vol) of animal-derived components, less than about 2% (vol/vol) of animal-derived components, less than about 1% (vol/vol) of animal-derived components. Yet another embodiment is directed to the method of producing an engineered coronavirus spike protein, where the method comprises a culturing step in a culture medium, where the culture medium can or cannot comprise an engineered animal origin free insulin. A further embodiment can provide for a method of producing an engineered coronavirus spike protein having a purity of about 95% or greater, about 96% or greater, about 97% or greater, about 98% or greater, about 99% or greater, about 99.2% or greater, about 99.4% or greater, about 99.6% or greater, about 99.8% or greater, where the purity of the engineered coronavirus spike protein can be substantially or essentially free from components, which: naturally accompany the engineered coronavirus spike protein, are used to produce the engineered coronavirus spike protein, or are degradation products from producing the engineered coronavirus spike protein. Contaminant components can include those materials that differ from the desired engineered coronavirus spike protein, or those naturally occurring or present materials, which would interfere with research, diagnostic, or therapeutic uses for the protein, and can include enzymes, hormones, and other proteinaceous or non-proteinaceous solutes. The purity of the engineered coronavirus spike protein described here can be determined by any art-recognized method of analysis (e.g., polyacrylamide gel electrophoresis, HPLC, analytical size exclusion chromatography (SEC), silver-stained gel, and the like). Generally, the purity of the engineered coronavirus spike protein means that the protein has been increased in purity, such that it exists in a form that is purer than when in its natural environment and/or when initially produced and/or synthesized. Typically, the purity of an isolated engineered coronavirus spike protein can be about 60% or greater (e.g., 70%, 80%, 90%, 92%, 93%, 94%, 95%, 96%, 97%, 98%, 99%.

### Coronavirus Spike Protein Product Characterization and Activity

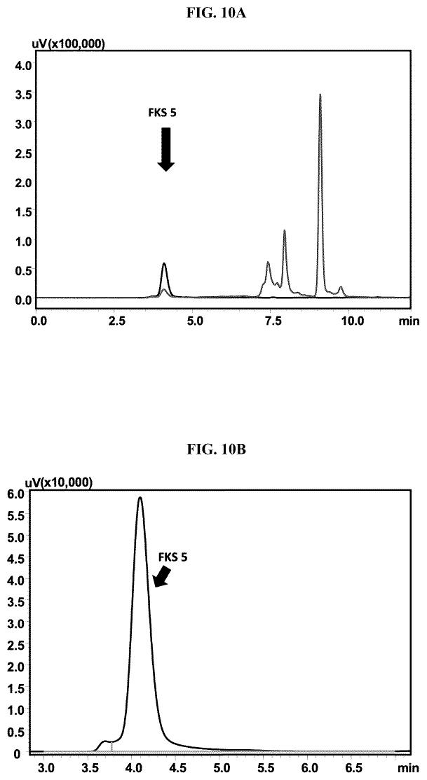

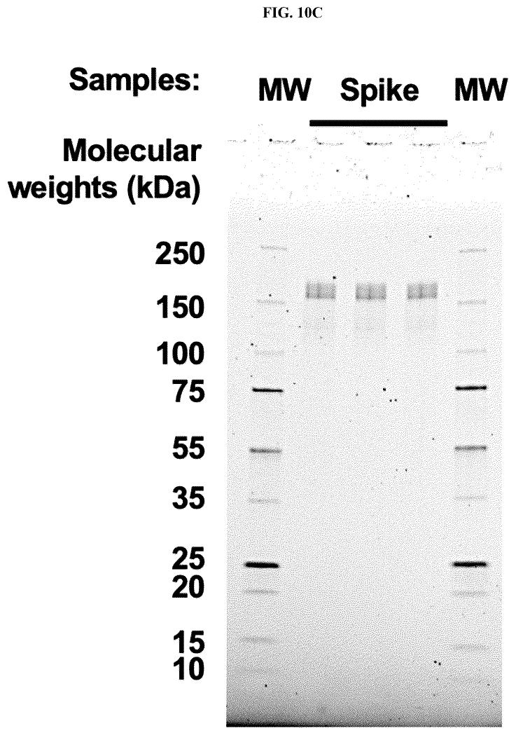

Engineered coronavirus spike protein can be purified from harvested cell culture fluids by a 2-step chromatographic procedure and subsequently analyzed by SEC. FIG. 10A shows the purified engineered coronavirus spike protein material fractions from supernatants of harvested cell culture fluids eluting at about 4.1 minutes-4.3 minutes, where the remaining peaks include cell culture contaminants. An expanded view of the main peak eluting at about 4.1 minutes-4.3 minutes is shown in FIG. 10B.

Another embodiment can be directed to an engineered coronavirus spike protein that has benefits over the wild-type coronavirus spike protein. For example, the disclosed engineered coronavirus spike protein incorporates mutations found in multiple coronavirus variants, which can convey broad cross-reactivity in a composition or vaccine for use in prophylactically treating a coronavirus infection or disease associated with a coronavirus infection. Other advantages of the engineered coronavirus spike protein described here also include stability and manufacturability (e.g., proline at positions 817, 892, 899, 942, 986, 987 (i.e., P817, P892, P899, P942, P986, and P987) relative to the modified sequence of FIG. 3E; a non-functional protease cleavage site at amino acid positions 682-685 relative to the modified amino acid sequence of FIG. 3E (e.g., a non-functional furin cleavage site of amino acid sequence GSAS (SEQ ID NO: 28)); replacement of a transmembrane domain and an intracellular tail of a wild-type or unmodified coronavirus spike protein with a T4 foldon motif also known as a T4 fibritin trimerization foldon motif (e.g., GYIPEAPRDGQAYVRKDGEWVLLSTFL (SEQ ID NO: 3) at amino acid positions 1211-1237 relative to the modified amino acid sequence of FIG. 3E)), which is greater than that found in the wild-type coronavirus spike protein. Mutations that prevent or inhibit protease cleavage, including for example, furin and the like, or render protease cleavage non-functional, are an additional advantage. Accordingly, variant engineered coronavirus spike proteins having amino acid mutations that increase protease inhibitory activity are useful in embodiments of the disclosure.

### Cell Line and Process Optimization

Embodiments show that a reliable, large-scale supply of high-quality coronavirus spike protein can be provided by engineered mammalian cells in bioreactors that are optimized for high-yield productivity. The engineered CHO cell lines or modified CHO cells, adapted to suspension culture, described here provide a basis for large-scale manufacturing, since the high-yielding small- to mid-scale processes developed here are scalable to about 1,000 L and 10,000 L operations. Thus, the production of hundreds of kilograms of modified CHO cell-derived engineered coronavirus spike protein can become a reality; whereas, in research and development, the supply of such engineered coronavirus spike protein in large quantities and of high quality and purity have failed, possibly also for commercial reasons that did not provide a balance of cost in manufacturing and market economics.

The problem of a low yield and substandard quality of engineered coronavirus spike protein, and methods of producing the same is solved by embodiments of the present disclosure, for example those embodiments that provide methods for generating highly productive, clonally-derived cell lines from a fast-growing host cell and its derivatives, i.e., optimized and thus modified CHO cells adapted for suspension culture, and the use of cell line development approaches and process conditions using very rich non-human component-free media and/or feed supplements. Together, these methods can generate coronavirus spike protein-producing cell cultures resulting in volumetric yields of engineered coronavirus spike protein in fed-batch processes in the multi-gram/liter range, e.g., up to and including about 10 g/L or greater. In addition, but not limited to a specific characteristic of the produced engineered coronavirus spike protein, it is possible to modulate the culture medium and certain process conditions in such a way to generate large yields of the engineered coronavirus spike protein described here with modifications that enhance stability and manufacturability of the CHO-derived engineered coronavirus spike protein. For example, rendering the protease cleavage site non-functional prevents the cleavage of the coronavirus spike protein sequence into two separate S1 and S2 peptides; and deletion of the transmembrane domain enhances secretion in CHO cells. Maintaining six prolines at positions P817, P892, P899, P942, P986, and P987 of the modified amino acid sequence of FIG. 3E serves to enhance protein stability by enabling the engineered coronavirus spike protein to remain in its pre-fusion state and form a non-covalently bound trimer, which is secreted in that form from the CHO cells described here. (See, e.g., WO 2021/243122 regarding proline stability, incorporated by reference in its entirety.)

Embodiments of the disclosure are directed to the materials and methods for generating or producing engineered coronavirus spike protein preparations useful for prophylactically treating coronavirus infection or diseases associated with coronavirus infection. The coronavirus spike protein produced by the materials and methods described here can include an engineered coronavirus spike protein having increased stability and capability for manufacturability resulting from the engineered coronavirus spike protein. Because of increased demands for coronavirus spike protein products, there has been a long desired and active interest in obtaining or producing high quality engineered coronavirus spike protein, and in large quantities, for human therapeutic use. The methods disclosed here allow for the production of high purity engineered coronavirus spike protein made in modified Chinese Hamster Ovary (CHO) cells cultivated in a bioreactor, where large scale production can occur, and the use of the produced engineered coronavirus spike protein in the prophylactic treatment of human disease or conditions associated with coronavirus infections or coronavirus infection itself, including variants thereof.

In one embodiment, clonally-derived cell lines comprising the desired engineered coronavirus spike protein can be cultivated under high-throughput conditions. For example, the clonally-derived cell lines described here can be cultivated under numerous conditions using a high-throughput culture system while applying small scale (10 ml) cultures in orbitally shaken 50 ml-OrbShake tubes (e.g., TPP® TubeSpin® bioreactor tubes, TubeSpin® Bioreactor 50, Trasadingen, Switzerland; or similar product). These tubes are provided with a ventilated cap and are typically shaken at 180 rpm with a displacement radius of 50 mm within a CO₂-controlled, humidified incubator shaker (Kuhner Shaker, Birsfelden, Switzerland). In one embodiment, the clonally-derived cell lines, comprising clonally-derived cells, can be cultivated under high-throughput conditions where cell viability and growth under use of numerous media compositions, feed compositions, timing of the additions, volumes of feed additions, temperature shifts, and other process conditions were studied. In another embodiment, the clonally-derived cells can be cultivated under high-throughput conditions and processed on a larger scale, where it is understood that the conditions for scaling up the process from small scale to large scale is direct or essentially direct. An example of various procedures useful in producing the desired engineered coronavirus spike protein can include the use of different feed strategies, e.g., feed volumes, feed types, feed timings, etc. and temperature shifts in a fed-batch process. See, TABLE 1. The feeds (7A and 7B; HyClone™ Cell Boost 7a, HyClone™ Cell Boost 7b, Catalog Numbers SH31026.01 (RRG168030, SH31027.01) are commercially available and are given in volumes in percent of the total effective cell culture volume and are provided every day (ED) or every other day (EOD).

TABLE 1

| | | | | Temperature Shift |
|---|---|---|---|---|
| # | Day | 7A (%) | 7B (%) | over Days (d) |
| 1 | EOD | 7.1 | 0.71 | d 00-d 03: 37° C. |
| 2 | EOD | 3.6 | 0.36 | d 03-d 17: 33° C. |
| 3 | ED | 3.6 | 0.36 | |
| 4 | ED | 1.8 | 0.18 | |
| 5 | EOD | 7.1 | 0.71 | d 00-d 03: 37° C. |
| 6 | EOD | 3.6 | 0.36 | d 03-d 05: 33° C. |
| 7 | ED | 3.6 | 0.36 | d 05-d 17: 31° C. |
| 8 | ED | 1.8 | 0.18 | |
| 9 | EOD | 7.1 | 0.71 | d 00-d 05: 37° C. |
| 10 | EOD | 3.6 | 0.36 | d 05-d 17: 31° C. |
| 11 | ED | 3.6 | 0.36 | |
| 12 | ED | 1.8 | 0.18 | |

Yet another embodiment can be directed towards the large-scale cell culturing and optimization using a bioreactor. In one embodiment, the use of chemically-defined feeds, such as a feed comprising inorganic salts, amino acids, and vitamins (e.g., XLG Feed A (Feed A4CHO); ExcellGene S.A.; etc.) and a feed comprising organics and other beneficial components (e.g., XLG Feed B (Feed B4CHO); ExcellGene S.A., etc.) can be added to the bioreactor at different time points during the production phase. These feeds can be added in the same or different volumes, i.e., fractions of the working volume of a production bioreactor. A person of ordinary skill in the art would understand how to modify process conditions and amounts in order to produce a defined quality of engineered coronavirus spike protein in large amounts.

A further embodiment provides a method of producing engineered coronavirus spike protein comprising: culturing or growing clonally-derived cells expressing engineered coronavirus spike protein; feeding clonally-derived cells expressing engineered coronavirus spike protein with at least one feed, where the feeding occurs continuously or discontinuously, where feeding can occur using controlled flow-rates; maintaining a cell culture temperature; shifting the maintained cell culture temperature to a shifted cell culture temperature; growing the cells at the decreased cell culture temperature until the cells express engineered coronavirus spike protein at a titer of about 500 mg/L or greater (e.g., 600 mg/L, 700 mg/L, 800 mg/L, 900 mg/L, 1 g/L, 2 g/L, 3 g/L, 4 g/L, 5 g/L, 6 g/L, 7 g/L, 8 g/L, 9 g/L, 10 g/L); or about 10.5 g/L or less (e.g., 9.5 g/L, 8.5 g/L, 7.5 g/L, 6.5 g/L, 5.5 g/L, 4.5 g/L, 3.5 g/L, 2.5 g/L, 1.5 g/L, 950 mg/L, 850 mg/L, 750 mg/L, 650 mg/L, 550 mg/L); or ranging from about 500 mg/L to about 10 g/L (e.g., about 600 mg/L to about 10 g/L, about 700 mg/L to about 10 g/L, about 800 mg/L to about 10 g/L, about 900 mg/L to about 10 g/L, about 1 g/L to about 10 mg/L, about 2 g/L to about 10 g/L, about 3 g/L to about 10 g/L, about 4 g/L to about 10 g/L, about 5 g/L to about 10 g/L, about 6 g/L to about 10 g/L, about 7 g/L to about 10 g/L, about 8 g/L to about 10 g/L, about 9 g/L to about 10 g/L). In a further embodiment, the clonally-derived cells are fed every day. Yet another embodiment can be directed to feeding the clonally-derived cells every other day. A further embodiment can be directed to feeding the clonally-derived cells every 3 days, or any other feeding schedule that benefits the overall health of the cells and thereby increases the final harvested product titer of engineered coronavirus spike protein.

One embodiment can be directed to growing clonally-derived cells expressing engineered coronavirus spike protein for a number of days sufficient to reach an engineered coronavirus spike protein titer of about 500 mg/L to about 10 g/L. The number of days sufficient to obtain an engineered coronavirus spike protein titer of about 500 mg/L or greater can range from 7 days to 21 days, or the number of days can be at least 7 days, at least 11 days, at least 14 days, at least 17 days, or at least 21 days.

In a further embodiment, the cell culture temperature during the production phase can be maintained at a temperature ranging from about 35° C. to about 38° C., including but not limited to, at about 35° C., at about 36° C., at about 37° C., at about 38° C., or less than about 39° C. Another embodiment can be directed to a shifted cell culture temperature ranging from about 24° C. to about 34° C., or any temperatures at or in between, including but not limited to, at about 24° C., at about 25° C., at about 26° C., at about 27° C., at about 28° C., at about 29° C., at about 30° C., at about 31° C., at about 32° C., at about 33° C., or at about 34° C. Another embodiment can be directed to a cell culture temperature that is maintained at about 37° C. for the first 2 days or the first 3 days, or portions of day 3 thereof. A further embodiment can be directed to a decreased cell culture temperature of about 33° C., about 32° C., about 31° C., about 30° C., about 29° C., about 28° C., about 27° C., about 26° C., about 25° C., or about 24° C. to about 25° C., where the decreased cell culture temperature occurs on day 3, or a portion of day 3 thereof. Yet, a further embodiment can be directed to a decreased cell culture temperature of about 31° C. occurring on day 5, or a portion of day 5 thereof. In another embodiment, the decreased cell culture temperature comprises more than one decreased cell culture temperature, where a first decreased cell culture temperature of about 33° C., about 32° C., about 31° C., about 30° C., about 29° C., about 28° C., about 27° C., about 26° C., about 25° C., or about 24° C. to about 25° C. occurs on day 3, or a portion of day 3 thereof, and a second decreased cell culture temperature occurs on day 5, or portions of day 5 thereof by about 2° C. to about 3° C. below the previously used temperature after the first temperature shift. Alternative days to those mentioned here are also contemplated for the temperature shift to occur, as long as the clonally-derived cells expressing engineered coronavirus spike protein are healthy, i.e., not dying or dead.

In another embodiment, the cell culture during the production phase comprising host cells incorporated with the engineered coronavirus spike protein described here, can be maintained to have an osmolarity ranging of about 200 mOsm/kg to about 600 mOsm/kg, about 250 mOsm/kg to about 400 mOsm/kg, about 260 mOsm/kg to about 320 mOsm/kg, about 450 mOsm/kg to about 600 mOsm/kg, about 500 mOsm/kg or greater, about 550 mOsm/kg or greater, or any appropriate osmolarity which maintains or promotes normal, healthy cells for achieving the desired protein levels of the gene of interest, including for example, engineered coronavirus spike protein. Changes in osmolarity over the days in culture are also contemplated as osmolarity increases during the course of cell culture and nutrients in fed-batch cultures also increase the osmolarity. Another embodiment can be directed to increasing osmolarity of the cell culture to about 550 mOsm/kg or greater by or at Day 5 or later, or any appropriate day that allows for normal, healthy cells for achieving the desired protein levels of the gene of interest, including for example, engineered coronavirus spike protein as described here. In some embodiments of the disclosure, oxygen can be provisioning to the cells in culture, or, under use of certain bioreactor systems, such as orbitally shaken bioreactors, purified air can instead be used exclusively, under avoidance of oxygen, during the cell culture production phase (see, e.g., Zhu, L. et al. Fluid dynamics of a pilot-scale OrbShake bioreactor under different operating conditions. J. Chem. Techn. Biotechnol. 2021, DOI 10.1002/jctb.6995, which is incorporated by reference for the teachings of the cell culture conditions).

Yet a further embodiment can be directed to a feed that is animal component-free, or chemically defined, optimized for high-yield protein production, including but not limited to, in fed-batch processes, free of growth factors, animal tissue-derived peptides, animal tissue-derived hydrolysates, phenol red, or 2-mercaptoethanol. In yet another embodiment, the feed can comprise of a neutral or close to neutral pH, i.e., a neutral feed, where the neutral feed can, in some embodiments, contain amino acids, vitamins, salts, and glucose. In a further embodiment, the feed can be chemically defined, or can contain non-animal derived components, such as hydrolysates from plant seeds, from certain cereals (wheat), from certain beans or peas (soybean) or the like. In a further embodiment, the feed can comprise of an alkaline pH, i.e., an alkaline feed, where the alkaline feed can, in some embodiments, contain a concentrated solution of amino acids. Another embodiment can be directed to feeding the clonally-derived cells expressing engineered coronavirus spike protein with a combination of feeds, where the combination of feeds can include the neutral feed and the alkaline feed, fed either simultaneously, essentially simultaneously, sequentially, or essentially sequentially. Moreover, feeding the cell culture with additives, including but not limited to, feeds, nutrients, amino acids, and the like, can occur continuously or discontinuously, where feeding can occur using controlled flow-rates, and a schedule comprising feeding at least one feed every day or every other day, or any other feeding schedule that maintains or promotes normal, healthy cells for achieving the desired protein levels of the gene of interest, including for example, engineered coronavirus spike protein.

Another embodiment can be directed to a combination of feeds, where the neutral feed has a concentration ranging from about 1% to about 8% of the total cell culture volume, or any percentages at or in between, including but not limited to, at about 1.8%, at about 3.6%, or at about 7.1%; and where the alkaline feed has a concentration ranging from about 0.1% to about 0.8% of the total cell culture volume, or any percentages at or in between, including but not limited to, at about 0.18%, at about 0.36%, or at about 0.71%. Another embodiment can be directed to concentrations of feed, where the alkaline feed is in an amount of about one-tenth (1/10) the amount of the neutral feed. For example, the percentage of the neutral feed ranges from about 1.8% to about 7.1% and the percentage of the alkaline feed ranges from about 0.18% to about 0.71%, i.e., one-tenth of the percentage of the neutral feed, where the feed percentages are in relation to the total cell culture volume that can include at least one of: the cells, culture medium, feeds, and any other nutrients or additives for culturing the cells to maintain or produce normal, healthy cells for achieving the desired levels of the protein, for example, engineered coronavirus spike protein, encoded by the gene of interest.

In one embodiment, a method of producing engineered coronavirus spike protein can be directed to: growing clonally-derived cells expressing engineered coronavirus spike protein; feeding the clonally-derived cells expressing engineered coronavirus spike protein a feed comprising: a neutral feed, such as for example, 7A feed (HyClone™ Cell Boost 7a) and an alkaline feed, such as for example, 7B feed (HyClone™ Cell Boost 7b) every other day; maintaining a culture temperature of 37° C. from day 0 to day 3, including a portion of day 3, i.e., from greater than 0 hours (day 0) to 72 hours, 78 hours or 84 hours or any timing in between the provided hours (day 3) shifting the culture temperature to 33° C. on day 3, when the previously provided timing of the first temperature setting ends, purifying engineered coronavirus spike protein from the cells; and collecting the purified engineered coronavirus spike protein. In one embodiment, the concentration of the neutral feed is about 7.1% of the total volume of cell culture and the concentration of the alkaline feed is about 0.71% of the total volume of cell culture. An embodiment of the disclosure can be directed to feeding a feed comprising a combination of a neutral feed and an alkaline feed, where the feed is fed every other day to the clonally-derived cells expressing engineered coronavirus spike protein, where the cell culture temperature is maintained at about 37° C. from day 0 to day 3, or a portion of day 3 thereof, and where the decreased cell culture temperature is about 33° C. starting on day 3, or a portion of day 3 thereof. In another embodiment further to the feeding schedule described here is a media used to grow the clonally-derived cells expressing engineered coronavirus spike protein, where the media provide additional nutrients, amino acids, metals, and the like, which enhance the cell culture conditions, and can include, for example, a chemically defined medium such as XLG_E21_07 (ExcellGene SA). Further embodiments of the disclosure can include the XLG_E21_07 medium by modifying the concentration of several components, such as increasing the concentration of glucose, zinc, asparagine, glutamic acid, and phosphate, simply by, for example, adding stock solutions of higher concentrations in small volumes for adjustment. The concentrations of these exemplary five components can be modified according to the following ranges found in TABLE 2:

TABLE 2

| | CONCENTRATION | CONCENTRATION |
|---|---|---|
| COMPONENT | FROM | TO |
| Glucose | about 2 g/L | about 30 g/L |
| Zinc | about 0.1 mg/L | about 10 mg/L |
| Asparagine | about 500 mg/L | about 7000 mg/L |
| Glutamic Acid | about 100 mg/L | about 3000 mg/L |
| Phosphate | about 100 mg/L | about 3000 mg/L |

A further embodiment can be directed to a method of producing engineered coronavirus spike protein, comprising: culturing or growing clonally-derived cells expressing engineered coronavirus spike protein; feeding the clonally-derived cells expressing engineered coronavirus spike protein at least one feed including but not limited to: a neutral feed, such as for example, HyClone™ Cell Boost 7A feed and an alkaline feed, such as for example, HyClone™ Cell Boost 7B feed every day, where the alkaline feed is present in an amount of about 1/10 that of the neutral feed, and the amount (vol/vol) of the feeds are based on the total cell culture volume; maintaining a culture temperature of 37° C. from day 0 to day 3, whereby the inoculation of the production vessel with fresh cells from the “N-1” bioreactor (pre-culture or seed bioreactor) is defined as the start of day 0, and then shifting the culture temperature to 33° C. on day 3 (i.e, 72 hours after the start of the production culture); purifying engineered coronavirus spike protein expressed by the cells; and collecting the purified engineered coronavirus spike protein. In one embodiment, the concentration of the neutral feed is about 3.6% of the total volume of cell culture and the concentration of the Docket No.: 178225-010202/US alkaline feed is about 0.36% of the total volume of cell culture. An embodiment of the disclosure can be directed to feeding a feed comprising a combination of a neutral feed and an alkaline feed, where the feed is fed every day to the clonally-derived cells expressing engineered coronavirus spike protein, where the cell culture temperature is maintained at about 37° C. from day 0 to day 3, or a portion of day 3 thereof, and where the decreased cell culture temperature is about 33° C. starting on day 3, or a portion of day 3 thereof. The feed, either alone or in combination, that can be used in the methods and processes described here include, but are not limited to, those presented in the table of feeds, TABLE 3:

TABLE 3

| FEED | MANUFACTURER | CATALOG NO. |
|---|---|---|
| HyClone ™ Cell Boost 7a | HyClone ™ | SH31026.01 |
| | | (RRG168030) |
| HyClone ™ Cell Boost 7b | HyClone ™ | SH31027.01 |
| Cellvento ™ Feed-210 | Merck Millipore | 1.02488.0005 |
| Cellvento ™ Feed-220 | Merck Millipore | 1.02578.0003 |
| Cellvento ™ Feed-200 | Merck Millipore | 1.01883.0003 |
| Cellvento ™ 4Feed | Merck Millipore | 1.03796.0005 |
| L-Cyteine + L-Tyrosine | Merck Millipore | 1.02735.0100 |
| (Feed B) | | |
| BalanCD CHO FEED 1 | Irvine | 91127 |
| BalanCD CHO FEED 2 | Irvine | 91129 |
| BalanCD CHO FEED 3 | Irvine | 99471 |
| BalanCD CHO FEED 4 | Irvine | 94134 |
| Efficient Feed ™ A + | Thermo Fisher | A25023-04 |
| AGT (3X) | | |
| Efficient Feed ™ B + | Thermo Fisher | A25030-04 |
| AGT (3X) | | |
| Efficient Feed ™ C + | Thermo Fisher | A25031-04 |
| AGT (2X) | | |
| PowerFeed A | Sartorius | BE 02-044Q |
| CHO Xtreme Feed ™ | Sartorius | BE02-056Q |
| PowerFeed Advance | Sartorius | |
| Ex-Cell Advanced CHO Feed 1 | Sigma | 24367-1L |
| (with glucose) | | |
| Ex-Cell CHOZN Platform Feed | Sigma | 24331C-10L |
| EX-Cell ™ Hydrolysate | Sigma | 24700C-100G |
| Cell Boost TM 1 (R05.2) | GE | SH30584.02 |
| Cell Boost TM 2 (R15.4) | GE | SH30596.01 |
| Cell Boost TM 3 (JM 3.5) | GE | SH30825.01 |
| Cell Boost TM 4 (PS307) | GE | SH30857.01 |
| Cell Boost TM 5 (CN-F) | GE | SH30865.01 |
| Cell Boost TM 6 (CN-T) | GE | SH30866.01 |
| Feed A4CHO (CDM) | ExcellGene SA | |
| Feed B4CHO (CDM) | ExcellGene SA | |
| Feed 3CHO (ACF) | ExcellGene SA | |
| FlexiFeed ® (CDM) | ExcellGene SA | |

In another embodiment further to the feeding schedule described here are media used to grow the clonally-derived cells expressing engineered coronavirus spike protein, where the media can be free of any components derived from animals, such as but not limited to fetal bovine serum (FBS), for example a chemically defined medium, such as medium XLG_E21_07 (ExcelGene SA). Non-limiting examples of media, either alone or in combination, useful in the methods and processes of producing engineered coronavirus spike protein described herein include also those presented in the table of media, where chemically derived (CD) and non-human animal component-free (ACF) are specified, see TABLE 4:

TABLE 4

| | CD/ | | CATALOG |
|---|---|---|---|
| MEDIUM | ACF | MANUFACTURER | NO. |
| CD CHO Medium (1x), | CD | GIBCO (Invitrogen) | 10743-011 |
| liquid | | | |
| CD FortiCHO | CD | GIBCO (Invitrogen) | A11483-01 |
| CD OptiCHO ™ | CD | GIBCO (Invitrogen) | 12681-029 |
| Medium | | | |
| HyClone ™ CDM4 | CD | HyClone ™ | SH30871.02 |
| PERMAb | | (GE Healthcare) | |
| HyClone ™ CDM4CHO | CD | HyClone ™ | SH30558.02 |
| | | (GE Healthcare) | |
| HyClone ™ CDM4MAb | CD | HyClone ™ | SH30802.02 |
| | | (GE Healthcare) | |
| EX-CELL ™ CD CHO | CD | SAFC Biosc | 14365C |
| Fusion | | (SIGMA) | |
| ActiPro | CD | HyClone ™ | SH31039.02 |
| | | (GE Healthcare) | |
| CD 293 | CD | GIBCO (Invitrogen) | 11913-01 |
| PowerCHO1 CD | CD | Lonza | 12-770Q |
| PowerCHO2 CD | CD | Lonza | BE12-771Q |
| PowerCHO3 CD | CD | Lonza | 12-772Q |
| ProNSO 1 | CD | Lonza | 12-773 Q |
| ProNSO 2 CD | CD | Lonza | 12-774Q |
| POWER CHO Advance | CD | Lonza | 12-927Q |
| CD DG 44 medium | CD | GIBCO (Invitrogen) | 12610-010 |
| PeproGrow CD-CHO | CD | PEPROTECH | AF-CD-CHO |
| Medium | | | |
| EX-CELL ™ CD CHO3 | CD | SAFC Biosc | C1490-1L |
| Medium | | (SIGMA) | |
| EX-CELL ™ | CD | SAFC Biosciences | 14366C-1000 |
| Advanced ™ CHO | | (SIGMA) | ML |
| Feed- batch Medium | | | |
| IS CHO CD XP | CD | Irvine | 91120 |
| BalanCD ™ CHO | CD | Irvine | 91128 |
| GROWTH A | | | |
| EX-CELL ™ CHOZN | CD | SAFC Biosci | 14330C-1L |
| Platform Medium | | (SIGMA) | |
| Hycell CHO Medium | CD | HyClone ™ | SH30934.01 |
| | | (GE Healthcare) | |
| Dynamis ™ Medium | CD | GiBCO (Invitrogen) | A26615-01 |
| Cellvento ™ CHO-100 | CD | Merck Millipore | 1.00899.0010 |
| Cellvento ™ CHO-200 | CD | Merck Millipore | 1.01885.0010 |
| Cellvento ™ CHO-210 | CD | Merck Millipore | 1.02485.0010 |
| Cellvento ™ CHO-220 | CD | Merck Millipore | 1.02680.0500 |
| Pro293s-CDM | CD | Lonza | BE02-025Q |
| CD Hybridoma | CD | GIBCO (Invitrogen) | 11279-023 |
| EX-CELL ™ CD | CD | SAFC Biosciences | H4409-1L |
| Hybridoma Medium | | (SIGMA) | |
| CHOMACS CD | CD | Miltenyi Biotec | 170-077-001 |
| | | GmbH | |
| SHEFF-CHO CD | CD | Kerry | S-870392 |
| COMPLETE | | | |
| ASF CHO 3.2 | CD | AJINOMOTO | |
| FlexiCHO ® | CD | ExcellGene | |
| XLG_E21_7 CDM | CD | ExcellGene | |
| EX-CELL ™ 620-HSF | ACF | SAFC Biosc | 14621C |
| Hybridoma | | (SIGMA) | |
| EX-CELL ™ ACF CHO | ACF | SAFC Bios (SIGMA) | C5467 |
| medium | | | |
| EX-CELL ™ CHO | ACF | SAFC Biosc(SIGMA) | C8862 |
| DHFR-medium | | | |
| Animal Component Free | | | |
| EX-CELL ™ 325 PF | ACF | SAFC Biosc(SIGMA) | 14340C |
| CHO | | | |
| EX-CELL ™ 302 SF | ACF | SAFC Biosc(SIGMA) | 14324C |
| for CHO | | | |
| Adenovirus expression | ACF | GIBCO (Invitrogen) | 12582-011 |
| medium (AEM) | | | |
| FreeStyle CHO | ACF | GIBCO (Invitrogen) | 12651-014 |
| expression | | | |
| Hybridoma-SFM serum- | ACF | GIBCO (Invitrogen) | 12045-084 |
| free | | | |
| Protein Expression | ACF | GIBCO (Invitrogen) | 12661-013 |
| Medium (PEM) | | | |
| HyQ ADCF MAB | ACF | HyClone ™ | SH 30349.02 |
| | | (Socochim) | |
| ProCHO5 | ACF | Lonza | 4SP144 |
| PowerCHO -GS | ACF | Lonza | BE12-776Q |
| EX-CELL ™ 293 | ACF | SAFC Biosc (SIGMA) | 14571C |
| EX-CELL ™ CHO5 | ACF | SAFC Biosc (SIGMA) | C0363-1L |
| Medium | | | |
| EX-CELL ™ GTM-3 | ACF | SAFC Biosc (SIGMA) | G9916 |
| ACF | | | |
| FreeStyle ™ 293 | ACF | GIBCO (Invitrogen) | 12338-018 |
| Expression | | | |

Another embodiment can be directed to the use of various media and/or feed compositions and any suitable combinations thereof, as well as combinations of the media and feed combinations with any other process condition including but not limited to certain defined pH values, oxygen level and/or oxygen-providing sparging (gas-providing) regimen (oxygen and/or air, with or without providing nitrogen and/or CO₂ simultaneously), temperature, and osmolarity that can result in an increase in the volumetric productivity of engineered coronavirus spike protein in cell culture to a titer of greater than or equal to about 1 g/L (e.g., 2 g/L, 4 g/L, 6 g/L, 8 g/L, 10 g/L).

Briefly, the pH value of the culture conditions during the production phase can range from about pH 6.25 to about pH 7.5; about pH 6.5 to about pH 7.3; about pH 6.7 to about pH 7.3; about pH 6.7 to about pH 7.1; about pH 6.8 to about pH 7; greater than or equal to about pH 6.5; greater than or equal to about pH 6.7; greater than or equal to about pH 6.8; less than or equal to about pH 7.5; less than or equal to about pH 7.3; less than or equal to about pH 7.1; or less than or equal to about pH 7. The oxygen level and/or sparging regimen of the culture conditions can range from about 20% oxygen relative to air saturation to about 60% oxygen relative to air saturation; about 25% oxygen relative to air saturation to about 55% oxygen relative to air saturation; about 20% oxygen relative to air saturation to about 60% oxygen relative to air saturation; greater than or equal to about 20% oxygen relative to air saturation; greater than or equal to about 25% oxygen relative to air saturation; greater than or equal to about 30% oxygen relative to air saturation; greater than or equal to about 35% oxygen relative to air saturation; greater than or equal to about 40% oxygen relative to air saturation; greater than or equal to about 45% oxygen relative to air saturation; greater than or equal to about 50% oxygen relative to air saturation; greater than or equal to about 55% oxygen relative to air saturation; less than or equal to about 60% oxygen relative to air saturation; less than or equal to about 55% oxygen relative to air saturation; less than or equal to about 50% oxygen relative to air saturation; less than or equal to about 45% oxygen relative to air saturation; less than or equal to about 40% oxygen relative to air saturation; less than or equal to about 35% oxygen relative to air saturation; less than or equal to about 30% oxygen relative to air saturation; or less than or equal to about 25% oxygen relative to air saturation. The temperature of the culture conditions can also range from about 26° C. to about 38° C.; about 28° C. to about 38° C.; about 29° C. to about 37° C.; about 31° C. to about 37° C.; about 34° C. to about 37° C.; about 31° C. to about 33° C.; greater than or equal to about 26° C.; greater than or equal to about 28° C.; greater than or equal to about 31° C.; greater than or equal to about 33° C.; greater than or equal to about 34° C.; greater than or equal to about 37° C.; less than or equal to about 38° C.; less than or equal to about 37° C.; less than or equal to about 36° C.; less than or equal to about 34° C.; less than or equal to about 33° C.; or less than or equal to about 31° C.

In one embodiment, a method for producing an engineered coronavirus spike protein comprises culturing a host cell with a first nucleic acid sequence encoding an engineered coronavirus spike protein, wherein the culturing step occurs at a first period of time at a first temperature and at a second period of time at a second temperature, and optionally at a third period of time at a third temperature. Another embodiment of the method can provide for the second temperature that is less than the first temperature (e.g., lower by at least: about 1° C., about 2° C., about 3° C., about 4° C., about 5° C., about 6° C., about 8° C., about 10° C., etc.). A further embodiment of the method can provide for the third temperature that is less than either the second temperature or the first temperature (e.g., lower by at least: about 1° C., about 2° C., about 3° C., about 4° C., about 5° C., about 6° C., about 8° C., about 10° C., etc.). Another embodiment of the method provides for the first temperature, the second temperature, and/or the third temperature that is greater than room temperature (e.g., about 15° C., about 20° C., about 21° C., about 22° C., about 23° C., about 24° C., about 25° C., etc.). In yet another embodiment, the first period of time of culturing can be for at least about 1 day, about 2 days, about 3 days, about 4 days, about 5 days, about 6 days, about 10 days, about 15 days, about 1-2 days, about 1-3 days, about 1-4 days, about 1-5 days, about 1-7 days, about 1-10 days, about 1-15 days, about 1-16 days, about 1-17 days, about 1-18 days, about 1-20 days, etc. A further embodiment provides for the second period of time of culturing, where the second period of time can be for at least about 1 day, about 2 days, about 3 days, about 4 days, about 5 days, about 6 days, about 10 days, about 15 days, about 1-2 days, about 1-3 days, about 1-4 days, about 1-5 days, about 1-7 days, about 1-10 days, about 1-15 days, about 1-16 days, about 1-17 days, about 1-18 days, about 1-20 days, etc. Yet another embodiment can be directed to a third period of time of culturing, where the third period of time can be for at least about 1 day, about 2 days, about 3 days, about 4 days, about 5 days, about 6 days, about 10 days, about 15 days, about 1-2 days, about 1-3 days, about 1-4 days, about 1-5 days, about 1-7 days, about 1-10 days, about 1-15 days, about 1-16 days, about 1-17 days, about 1-18 days, about 1-20 days, etc. A further embodiment can be directed to the first period of time comprising about 1-20 days, about 1-18 days, about 1-17 days, about 1-16 days, about 1-15 days, about 1-10 days, about 1-6 days, about 1-5 days, about 1-4 days, about 1-3 days, or about 1-2 days; the second period of time comprising about 1-20 days, about 1-18 days, about 1-17 days, about 1-16 days, about 1-15 days, about 1-10 days, about 1-6 days, about 1-5 days, about 1-4 days, about 1-3 days, or about 1-2 days; and optionally, a third period of time comprising about 1-20 days, about 1-18 days, about 1-17 days, about 1-16 days, about 1-15 days, about 1-10 days, about 1-6 days, about 1-5 days, about 1-4 days, about 1-3 days, or about 1-2 days.

Yet a further embodiment can provide for a method for producing a human engineered coronavirus spike protein comprises culturing a host cell with a first nucleic acid sequence encoding a human engineered coronavirus spike protein, wherein the culturing step occurs at a first period of time at a first temperature and at a second period of time at a second temperature, wherein a first feed and a second feed are administered every other day or every day, the first period of time comprises about 1-10 days, about 1-6 days, about 1-5 days, about 1-4 days, about 1-3 days, about 1-2 days at a first temperature ranging from about 31° C. to about 37° C., about 33° C. to about 37° C., and the second period of time comprises about 1-20 days, about 1-18 days, about 1-17 days, about 1-15 days, about 1-10 days, about 1-8 days, about 1-7 days, about 1-6 days, about 1-5 days, about 1-4 days, about 1-3 days, about 1-2 days at a second temperature ranging from about 31° C. to about 37° C., about 33° C. to about 37° C., and if the method comprises a third period of time at a third temperature, the third period of time comprises about 1-20 days, about 1-18 days, about 1-17 days, about 1-15 days, about 1-10 days, about 1-8 days, about 1-7 days, about 1-6 days, about 1-5 days, about 1-4 days, about 1-3 days, about 1-2 days at a third temperature ranging from about 31° C. to about 37° C., about 33° C. to about 37° C. In some embodiments, a second nucleic acid sequence or vector encoding the transposase DNA is co-transfected with the first nucleic acid sequence or vector, where the second nucleic acid sequence or vector does not integrate into the genome of the cells, but transiently provides the transposases that mediate integration of the Spike protein expression cassette into the genome. Such embodiments using the two nucleic acid sequences or vectors are for generating the recombinant cell populations before the production phase and undergo such culturing conditions as described here.

Thus, one solution to the problem of insufficient supply of engineered coronavirus spike protein was directed to embodiments for producing very high yields from engineered mammalian cells expressing engineered coronavirus spike protein. In embodiments of the disclosure, process conditions for growth and productivity while taking into consideration the diverse physiological metabolism of clonally derived cell populations used and media and feed compositions that support advantageous phenotypes of cells for bioreactor-based manufacturing of proteins were identified.

### Engineered Coronavirus Spike Protein Compositions and Vaccinations Therewith

Some embodiments provide a pharmaceutical composition comprising: a protein comprising an engineered coronavirus spike protein as described here (e.g., FIGS. 2-4) or a nucleic acid molecule encoding such engineered coronavirus spike proteins (e.g., FIG. 6), where the pharmaceutical composition comprises a pharmaceutically acceptable vehicle (e.g., carrier, diluent, excipient, adjuvant). Additional embodiments provide for such pharmaceutical compositions, where the pharmaceutical compositions are vaccines for prophylactically treating a coronavirus infection or disease associated with a coronavirus infection in a subject, such as, for example, a human subject. Some embodiments provide for use of any of the described proteins, nucleic acids, or pharmaceutical compositions or a method comprising prophylactically treating a coronavirus infection or disease associated with a coronavirus infection (e.g., COVID-19 or variant thereof (e.g., Wuhan; alpha; beta; gamma; delta; epsilon; eta; iota; lambda; mu; omicron BA.1; omicron BA.2; omicron BA.5; omicron BQ.1.1.; omicron XBB.1.5; zeta; and any future COVID-19 variants; and any combinations thereof)) in a subject. In one aspect, prophylactic treatment comprises administering to the subject an effective amount of any of the proteins described here, any of the nucleic acid molecules described here, or any of the pharmaceutical compositions comprising such proteins or such nucleic acid molecules, where the subject has not been vaccinated against a coronavirus infection, has not been infected by SARS-CoV-2, or has a spike protein antibody (e.g., spike protein immunoglobulin G (IgG)) level that is considered negative or non-neutralizing, for example, less than 2,500 units/mL (e.g., 2,000; 1,500; 1,000; 500; 250; 100; 50; 10; 5; 1). By administering to a subject who has not previously been vaccinated against coronavirus nor infected by the SARS-CoV-2 virus, a broad range of protection can be generated, i.e., protection against severe disease and/or against a future coronavirus infection by any past or newly emerging coronavirus variants.

Additional embodiments are directed to use of any of the described proteins, nucleic acids, or pharmaceutical compositions or a method comprising prophylactically treating a coronavirus infection or disease associated with a coronavirus infection (e.g., COVID-19 or variant thereof (e.g., Wuhan; alpha; beta; gamma; delta; epsilon; eta; iota; lambda; mu; omicron BA.1; omicron BA.2; omicron BA.5; omicron BQ.1.1.; omicron XBB.1.5; zeta; and any future COVID-19 variants; and any combinations thereof) by administering a booster vaccination in a subject. In one aspect, the booster vaccination comprises administering to the subject an effective amount of any of the proteins described here, any of the nucleic acid molecules described here, or any of the pharmaceutical compositions comprising such proteins or such nucleic acid molecules, where the subject has been administered any of the proteins, nucleic acid molecules, or pharmaceutical compositions described here, thereby being vaccinated against a coronavirus infection, where the subject has been infected by, for example, SARS-CoV-2. In other embodiments, the subject to whom such proteins, nucleic acid molecules, or pharmaceutical compositions are administered, can also include those subjects with a spike protein antibody (e.g., spike protein immunoglobulin G (IgG)) level that is considered negative or non-neutralizing, for example, less than 2,500 units/mL (e.g., 2,000; 1,500; 1,000; 500; 250; 100; 50; 10; 5; 1). By administering to a subject who has not previously been vaccinated against coronavirus nor infected by the SARS-CoV-2 virus, a broad range of protection can be generated, i.e., protection against severe disease and/or against a future coronavirus infection by any past or newly emerging coronavirus variants.

In some embodiments, such pharmaceutical compositions can also comprise an agent that boosts an immune response and enhances the effectiveness of a vaccine, i.e., an adjuvant. Non-limiting examples of an adjuvant include: aluminum salts, aluminum hydroxide, lipid/polymer-based nanoparticles, immunostimulating complex (Iscom, Matrix M), Alhydroxiquim-II, AS01, AS03, MF59 (squalene oil/water emulsion), CpG (CpG 1018), and the like. See, e.g., Counoupas C et al. Microbiol Spectr 10(1):e0169521, 2022, which is incorporated by reference herein in its entirety for teachings of adjuvants.

Additional embodiments can include a cell comprising a nucleic acid molecule encoding an engineered coronavirus spike protein described here. In some embodiments the cell is a host cell expressing any of the engineered coronavirus spike proteins disclosed here.

In further embodiments, a method comprising prophylactically treating a coronavirus infection or disease associated with a coronavirus infection in a subject, where prophylactically treating comprises administering to the subject an effective amount of: any of the engineered coronavirus spike proteins disclosed here, any of the disclosed nucleic acid molecules encoding the engineered coronavirus spike protein, or any of the pharmaceutical compositions comprising a pharmaceutically acceptable vehicle (e.g., carrier, diluent, excipient) and any of the engineered coronavirus spike proteins as described here or any of the disclosed nucleic acid molecules encoding the engineered coronavirus spike protein. Some embodiments are directed to methods of prophylactically treating a SARS-CoV-2 infection (e.g., COVID-19) or disease associated with COVID-19. In additional embodiments, the method prophylactically treats a coronavirus infection that is a COVID-19 variant selected from: Wuhan; alpha; beta; gamma; delta; epsilon; eta; iota; lambda; mu; omicron BA.1; omicron BA.2; omicron BA.5; omicron BQ.1.1.; omicron XBB.1.5.; zeta; and any future variants for which neutralizing antibodies are produced by the engineered coronavirus spike protein described here.

Some embodiments provide a use of any of the nucleic acid molecules encoding for such engineered coronavirus spike proteins as indicated in FIGS. 2-4, including for example, those in FIG. 6, or pharmaceutical compositions comprising any of these engineered coronavirus spike proteins or nucleic acid molecules as described here, for prophylactically treating a coronavirus infection or disease associated with a coronavirus infection. Some embodiments are directed to uses of prophylactically treating a SARS-CoV-2 infection (e.g., COVID-19) or disease associated with COVID-19. In additional embodiments, the uses for prophylactically treating a coronavirus infection are provided where the coronavirus infection is a COVID-19 variant selected from: Wuhan; alpha; beta; gamma; delta; epsilon; eta; iota; lambda; mu; omicron BA.1; omicron BA.2; omicron BA.5; omicron BQ.1.1.; omicron XBB.1.5.; zeta; or any future variants for which neutralizing antibodies are generated by the engineered coronavirus spike protein described here.

Embodiments described here provide for administration of compositions to subjects for preventing or prophylactically treating a coronavirus infection, a biologically compatible form, which can also be a vaccine, suitable for administration in vivo. The biologically compatible form comprises an active engineered coronavirus spike protein that can be administered in which any toxic effects are outweighed by the benefits of the active engineered coronavirus spike protein, which can include protecting a subject from getting seriously ill, hospitalized, or dying of the coronavirus infection as compared to a subject not having received a prophylactic treatment. An effective amount, at dosages and for periods of time to achieve the desired result can be utilized when administrating an effective amount of the composition comprising an engineered coronavirus spike protein, such as for example, an engineered coronavirus spike protein, as defined here, or nucleic acid molecule encoding such engineered coronavirus spike proteins. Factors such as age, sex, and weight of a subject or individual who can receive or to whom the composition can be administered comprising an engineered coronavirus spike protein, and the ability of coronavirus spike protein to elicit a desired immune response in the subject, are contemplated and considered for the amount of an active. These factors can also be considered for dosage purposes and as well as regimens that can be adjusted accordingly, thus providing the most optimum therapeutic response.

One embodiment can be directed to compositions, including pharmaceutical compositions, comprising an active engineered coronavirus spike protein described here in an appropriate pharmaceutically acceptable vehicle (e.g., carrier, diluent, or excipient), including but not limited to, water, saline, aqueous buffer, and the like, that are sufficiently sterile for administration (e.g., intravenous, subcutaneous, etc.). A subject can be prophylactically treated for a coronavirus infection by administering to the subject an effective amount of an engineered coronavirus spike protein or nucleic acid molecule encoding such engineered coronavirus spike proteins described here, and in one embodiment, an engineered coronavirus spike protein, where the amount is effective to prevent, ameliorate, or reduce a coronavirus infection in the subject. Prophylactically treating the subject and increasing the antibodies against coronavirus spike proteins in the subject to a level such that the subject has protection against a coronavirus infection, such as for example COVID-19.

Other embodiments of the disclosure provide for a method of using or use of the products (e.g., described engineered coronavirus spike proteins or nucleic acid molecules encoding such engineered coronavirus spike proteins, and pharmaceutical compositions comprising such proteins or nucleic acid molecules) as a booster vaccination of subjects who have previously been vaccinated against coronavirus infection or has previously been infected by the SARS-CoV-2 virus, in order to generate a long-lasting and broad range of protection against severe disease caused by a future infection by any emerging virus variants. In additional embodiments, the methods of using products (e.g., described engineered coronavirus spike proteins or nucleic acid molecules encoding such engineered coronavirus spike proteins, and pharmaceutical compositions comprising such proteins or nucleic acid molecules) are made of highly antigenic surface proteins from non-coronaviruses, such as but not limited to, Ebola, Marburg, Rous Sarcoma Virus (RSV), measles, and the like, that are made in Chinese hamster ovary (CHO) cells as highly purified antigen proteins for use in vaccination.

Dosage formulations, dosage amounts, and routes of administrating such dosage formulations can vary depending on the age and weight of the subject, etc. Alternatively, the dose can be increased or decreased based on the level to provide the best benefit for the subject to prevent or reduce coronavirus infection or a disease associated with coronavirus. In another embodiment, a booster can also be administered to the subject to improve an immune response. Compositions of the disclosure comprising an effective amount of an engineered coronavirus spike protein or variant thereof can be administered by any suitable route to humans or non-human animals as deemed appropriate by a physician. It will be appreciated that the amount of the engineered coronavirus spike protein or variant thereof according to this disclosure to be administered to the patient and required for use in prophylaxis according to the present disclosure will vary with the route of administration, the nature and severity of the condition for which treatment or prophylaxis is required, the age, weight, etc., and will be ultimately at the discretion of the attendant physician and/or manufacturer recommendations. In general, however, a useful amount such that by administration of the described pharmaceutical composition to the subject for prophylactically treating a coronavirus infection is sufficient to generate protective levels of neutralizing antibodies. For example, a pharmaceutical composition and pharmaceutically acceptable vehicle (e.g., carrier, diluent, excipient, adjuvant) comprising an engineered coronavirus spike protein as described here or nucleic acid molecule encoding such engineered coronavirus spike protein, or pharmaceutical composition comprising such proteins or nucleic acid molecules can be administered to a subject by any appropriate route (e.g., intramuscular, subcutaneous, intranasal, oral) in an amount sufficient to induce an immune response to the engineered coronavirus spike protein (e.g., 1 microgram/mL-500 micrograms/mL, about 10 micrograms/mL, about 15 micrograms/mL, about 50 micrograms/mL, 100 micrograms/mL, 200 micrograms/mL), where multiple doses over a period of time (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, etc) can be administered. However, subjects or individuals can be monitored, and dosages or regiments can be altered in order to provide the best outcome for the subject.

Another embodiment can be directed to compositions including pharmaceutical compositions and pharmaceutically acceptable vehicles (e.g., carriers, diluents, excipients, adjuvants), where the compositions can contain the engineered coronavirus spike protein described here, where the compositions can be administered internally (i.e., by injections, infusions, inhalations) for prophylactic treatment can be administered to a subject in need thereof, by for example parenteral administration, including but not limited to, intravenously, intracardiacally, intracoronarily, intramuscularly, subcutaneously, by inhalation, bronchial/tracheal instillation, dermally, intradermally, transdermally, intramucosally, transmucosally, topically, intranasally, and the like, and combinations thereof. Administration can also occur in tissues and cavities by a route including but not limited to intraperitoneally, intrapleurally, intrathecally, intraarterially, parenterally, and the like, and combinations thereof. Intramucosal administration can occur via mucous membranes, such as but not limited to, lips, mouth, nasal passages, middle ear, eustachian tube, the lining of the digestive tract, the lining of the urogenital tract (including the urethra and vagina), the lining of the respiratory tract, and eyes (including conjunctival membranes), which can include topical application as well as, for example, intravitreal injection. Depending on the route of administration, the active engineered coronavirus spike protein can be coated in a material to protect the compound from the degradation by enzymes, acids and other natural conditions. In one embodiment, the active engineered coronavirus spike protein, including engineered SARS-CoV-2 spike protein or compositions comprising the engineered coronavirus spike protein thereof, can be administered, for example, intramuscularly. In another embodiment, the active engineered coronavirus spike protein, including engineered SARS-CoV-2 spike protein or compositions comprising the engineered coronavirus spike protein or nucleic acid molecule encoding the engineered coronavirus spike protein thereof, can be administered intranasally, or by direct inhalation into the lungs. In yet a further embodiment, the active recombinant engineered coronavirus spike protein, including recombinant engineered coronavirus spike protein, or compositions comprising the recombinant engineered coronavirus spike protein thereof, can be administered topically. See, McMillan et al. Vaccines. 10(4):578, 2022.

In an embodiment of the invention, the active, recombinant engineered coronavirus spike protein thereof can be prepared in a composition for topical administration. Non-limiting examples of compositions comprising the recombinant engineered coronavirus spike protein derived from the modified CHO cells described here include a solution, a spray, a lotion, a gel, a cream, a balm, a paste, or an ointment.

A central role of disease burden can be caused by viral disease (e.g., in animals, of animal origin, with potential to spread to humans). Coronaviruses with their specific epidemic or pandemic dangers and challenges resulting in morbidity, mortality, and associated costs use proteases for cell entry and subsequent infection. In some embodiments of the disclosure, an engineered coronavirus spike protein, a nucleic acid molecule comprising such engineered coronavirus spike protein, or pharmaceutical compositions comprising a vehicle and such engineered coronavirus spike proteins or nucleic acid molecules encoding such coronavirus spike proteins thereof, that results in broad cross-reactivity sufficient to reduce or prevent coronavirus infections or diseases associated with coronavirus infections, where the infections are caused by SARS-CoV-2 or variants thereof.

A further embodiment can be directed to administration of the engineered coronavirus spike protein CHO-derived engineered coronavirus spike protein efficiently through skin or mucosa to access, for example, the lungs, the respiratory system, the circulatory system, and the like. In one embodiment, the engineered coronavirus spike protein can be inhaled.

The problem is furthermore solved by embodiments of the present disclosure that provide a method of prophylactically treating coronavirus infection or a disease associated with coronavirus infection, where the engineered coronavirus spike protein made in cultivated mammalian cells (e.g., modified CHO cells), is produced in vast quantities sufficient to prepare a vaccine.

Unless defined otherwise, all terms of art, notations and other technical and scientific terms or terminology used herein can be intended to have the same meaning as can be commonly understood by one of ordinary skill in the art to which the claimed subject matter pertains. In some cases, terms with commonly understood meanings can be defined herein for clarity and/or for ready reference, and the inclusion of such definitions herein should not necessarily be construed to represent a substantial difference over what can be generally understood in the art.

Throughout this application, various embodiments can be presented in a range format. It should be understood that the description in range format can be merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, the term “about” a number can refer to that number plus or minus 10% of that number. The term “about” a range can refer to that range minus 10% of its lowest value and plus 10% of its greatest value.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

Throughout this description, various components can be identified having specific values or parameters, however, these items are provided as exemplary embodiments. Indeed, the exemplary embodiments do not limit the various aspects and concepts of the present disclosure as many comparable parameters, sizes, ranges, and/or values can be implemented. Unless otherwise specified, the terms “first,” “second,” and the like, “primary,” “secondary,” and the like, do not denote any order, quantity, or importance, but rather are used to distinguish one element from another.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, etc.); for topical administration (e.g., as a cream, gel, lotion, or ointment); for intravenous administration (e.g., as a sterile solution free of particulate emboli and in a solvent system suitable for intravenous use); or in any other formulation described herein (see below).

As used herein, the phrase “pharmaceutically acceptable” generally safe for ingestion or contact with biologic tissues at the levels employed. Pharmaceutically acceptable is used interchangeably with physiologically compatible. It will be understood that the pharmaceutical compositions of the disclosure include nutraceutical compositions (e.g., dietary supplements) unless otherwise specified.

Unit dosage forms, also referred to as unitary dosage forms, often denote those forms of medication supplied in a manner that does not require further weighing or measuring to provide the dosage (e.g., vial, disposable single use container, pre-filled syringes, tablet, capsule, caplet, metered dose inhaler, inhaled dry powders, spray dose devices, intramuscular injection, etc.). For example, a unit dosage form can refer to a physically discrete unit suitable as a unitary dosage for human subjects and other mammals, each unit containing a predetermined quantity of active material (e.g., vaccine described here) calculated to produce the desired therapeutic effect, in association with any suitable pharmaceutical excipient or excipients. Exemplary, non-limiting unit dosage forms include a vial, a disposable single use container, a pre-filled syringe, a tablet (e.g., a chewable tablet), caplet, capsule (e.g., a hard capsule or a soft capsule), lozenge, film, strip, and gel cap. In certain embodiments, the compounds described herein, including crystallized forms, polymorphs, and solvates thereof, can be present in a unit dosage form. Some embodiments include multi-unit dosage forms, such as vials with multiple units that can be extracted via syringe for multiple administrations or vaccinations in a subject.

Useful pharmaceutical vehicles (e.g., carriers, excipients, diluents, adjuvants) for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions can be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringers solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

The term “effective amount” of an active (e.g., engineered coronavirus spike protein or nucleic acid molecule encoding such engineered coronavirus spike protein), as used herein, is that amount sufficient to effect beneficial or desired results, such as clinical results, and, as such, an “effective amount” depends upon the context in which it is being applied. In some embodiments, the compounds are administered in an effective amount for prophylactically treating a disease, disorder, or condition, such as a coronavirus infection. In another embodiment, in the context of administering an agent, an effective amount of an agent is, for example, an amount sufficient to achieve prevention or prophylaxis of one or more symptoms or conditions; diminishment of the extent of a disease, disorder, or condition; stabilized (i.e., not worsening) state of a disease, disorder, or condition; prevention of the spread of disease, disorder, or condition, whether detectable or undetectable, as compared to the response obtained without administration of the agent.

Determining an effective amount of an agent to be delivered can depend upon a number of factors including, for example, the biological activity of the agent, the age and weight of the animal (e.g., human), and the route of administration. The frequency of treatments or boosters depends upon a number of factors, such as the amount of the composition administered per dose, as well as the age, health and history of the subject, e.g., immunocompromising conditions. The precise amount, number of doses/boosters, and timing of doses/boosters will be determined by the attending physician, veterinarian, or manufacturer. Compositions of the present disclosure can be administered to any animal, including to mammals. Non-limiting examples of mammals include humans, non-human mammals, including but not limited to, dogs, cats, mice, rats, rabbits, sheep, birds, cattle, horses, and pigs.

Typically, the prophylactic treatment of a disease, disorder, or condition (e.g., the conditions associated with a disease) is an approach for obtaining beneficial or desired results, such as clinical results. Beneficial or desired results can include, but are not limited to, alleviation or amelioration of one or more symptoms or conditions; preventing the disease, disorder, or condition or spread thereof; or amelioration or palliation of the symptoms of a disease, disorder, or condition, whether detectable or undetectable.

As used herein, the term “subject” refers to any organism to which a composition and/or compound in accordance with the disclosure can be administered, e.g., for experimental, diagnostic, prophylactic, and/or therapeutic purposes. Typical subjects include any animal (e.g., mammals such as mice, rats, rabbits, non-human primates, and humans, etc.). A subject in need thereof is typically a subject for whom it is desirable to treat a disease, disorder, or condition as described herein. For example, a subject in need thereof can seek or be in need of treatment, require treatment, be receiving treatment, can be receiving treatment in the future, or a human or animal that is under care by a trained professional for a particular disease, disorder, or condition.

It is understood that aspects and embodiments of the disclosure described here include “comprising,” “consisting,” and “consisting essentially of” aspects and embodiments.

### EXAMPLES

The following examples illustrate specific aspects of the instant description. The examples should not be construed as limiting, as the example merely provides specific understanding and practice of the embodiments and its various aspects.

### Example 1

### Host Cell Expression System for Engineered Coronavirus Spike Protein

The modified CHO cells described here (i.e., fast growing, high protein yielding, high density robustness, scalability under suspension culture, etc.) were developed for bioreactor use, i.e., large-scale manufacturing, by modifying and selecting a specific Chinese hamster ovary (CHO) cell line that was originally generated in an academic laboratory (Puck T T, et al. J. Exptl. Med. 108(6):845-956, 1958) that had been subsequently provided to numerous researchers, including university groups, over the decades. CHO cells are known to change their genetic make-up rapidly and to adapt to various culture conditions in a manner that is very similar to cancer cells in humans or animals. The inventive cells were derived from a non-recombinant cell host, extensively optimized for rapid, robust growth under suspension culture in animal component-free media. These phenotypic features are inherited after transfection to the recombinant cells that express the engineered coronavirus spike protein (CoV) described here, and among other characteristics allow these cells to grow to a density of greater than 20 million cells/mL and a growth rate resulting in a doubling rate of less the 20 hours/cell doubling, yielding over about 2 g/L, over about 3 g/L, over about 4 g/L, over about 5 g/L, over about 6 g/L, over about 7 g/L, or over 8 g/L of engineered coronavirus spike protein, demonstrating the robustness and productivity in large-scale manufacturing, while growing in a non-human or an animal component-free media. Clonal CHO cell lines expressing recombinant human coronavirus spike protein were successfully established using a highly efficient transposon-based gene transfer technology described here. The convoluted history of CHO cells and their uncertain genetic makeup is described in a publication. (Wurm, F. M. Processes 1(3):296-311, 2013).

### Example 2

### Expression Vector System for Transfection

A two-vector co-transfection approach was used for the transfection of CHO cells with the nucleic acid sequence encoding the desired engineered coronavirus spike protein of interest. The desired engineered coronavirus spike protein was encoded by the gene of interest (GOI)-vector expressing engineered coronavirus spike protein (CoV), pXLG-6-CoV expression vector (ExcellGene S.A.). FIG. 7 shows the pXLG-6 vector map into which the engineered coronavirus spike protein gene was cloned within the multi-cloning site (MCS). A second plasmid vector, pXLG-5 (ExcellGene S.A.), encoded for the PiggyBac transposase (mPBase) (FIG. 8) was used in the two-vector co-transfection approach.

The gene of interest (i.e., encoding the engineered coronavirus spike protein) in the form of an intron-free DNA was cloned into a high-performance expression vector, for example, the pXLG6 expression vector. The gene of interest encoding the desired engineered coronavirus spike protein comprising about 1,240-1,249 amino acids was highly conserved among coronaviruses and for mediating infection into various animal species (Verma, J., Subbaro, N. (2021) A comparative study of human betacoronavirus spike proteins: structure, function and therapeutics. Arch. Virol. 166, 697-714).

### Example 3

### Transfection and Selection of Recombinant Engineered Coronavirus Spike Protein-Expressing CHO Cells

Non-recombinant host cells having the desired phenotype for rapid growth while in a non-human or an animal component-free media was used in generating a master cell bank (MCB), which was verified as being Chinese hamster ovary cells. The described efficient transposon-based gene transfer technology occurred by co-transfecting the host cells with a donor vector comprising the engineered coronavirus spike protein gene sequence and a second transposase expressing nucleic acid that mediated insertion of the engineered coronavirus spike protein gene into the genome of the host cells with the help of a transposase encoded by the transposase gene. Cells derived from a seed train culture under suspension culture in FlexiCHO® medium (ExcellGene) (established from the aforementioned Master Cell Bank) were spun down by centrifugation and transfected following strict instructions provided in the commercially provided transfection kit (CHO4Tx®; ExcellGene SA), providing 5 sg of the expression vector cocktail (comprising of the two aforementioned vectors) for a cell culture volume of 10 ml. Subsequently, transfected cells were cultured under suspension while shaking in a CO-controlled and humidified incubator-shaker at 180 rounds per minute and at 37° C. Each day, the cell culture was centrifuged and the pellet of cells was resuspended in fresh pre-warmed medium provided in the transfection kit (CHO4Tx®), containing 50 μg/ml of puromycin for selection. The viability and cell number of the culture decreased for about 4 days after transfection, but then the cell population began to recover and both viability and cell number began to increase. After 10 days under selective conditions, a highly viable population of healthy growing cells was re-established. No further puromycin selection of the established culture was executed. This rapidly growing, recombinant population of cells was shown to express human recombinant engineered coronavirus spike protein at high levels. This population of cells was considered a “pool” of recombinant engineered coronavirus spike protein cells, representing a mixture of multiple and diverse genetic integration events of the engineered coronavirus spike protein gene into the genome of the CHO cells.

An expansion of recombinant cell population, i.e., the recombinant CHO-CoV pool, was used to generate a Research Cell Bank (RCB-P-rCoV). A vial of this population was thawed, expanded using ExcellGene's FlexiCHO® medium, and single cell cloned and re-cloned a second time using a limiting dilution approach. This approach assured a clonal origin of the emerging cell populations with a probability of greater than 98%. The Research Cell Bank “RCB-P 03-rCoV” was used for the twice limiting dilution approach of single cells and delivered 72 highly productive clonally-derived cell populations, five (5) of which were further studied in long-term cultures. These long-term cultures were derived from additional Research Cell Banks, generated with the clonally-derived cell populations. The productivity of subcloned cells of these cell lines remained stable and the best performing clonally derived cell populations were frozen again with an indication of the corresponding clone name.

Cell lines are derived under batch and fed-batch conditions with a non-optimized process. Under these non-optimized cell culture production conditions clonally derived cell populations show expression levels of 500 mg/L or greater after a 14-day process, which was significantly higher than the yields obtainable using conventional methods.

A clonal cell line is developed from transfection and performed under similar conditions as described previously. The production conditions included the use of a diversity of media and feeds, without being optimized in any profound way, in 10 ml cultures maintained in 50 ml OrbShake™ tubes. Under certain studied conditions, product titers of 2 g/L-4 g/L or greater is obtained over 14 days. Following the schedule used in TABLE 1, at time points of 12 and 14 days, four different media conditions are compared (n=4)—use of an ExcellGene medium (XLG_E21_7 CDM) in combination with Feeds 7A/7B (HyClone™ Cell Boost 7a Supplement (SH31026.01); HyClone™ Cell Boost 7b Supplement (SH31027.01); GE Healthcare Life Sciences); HyClone CDM4CHO with Feeds 7A/7B (GE Healthcare Life Sciences); Ex-Cell® Advanced™ CHO Fed-batch medium from Sigma-Aldrich; and BalanCD is a medium from Irvine.

### Example 4

### Engineered Coronavirus Spike Protein Produced in Chinese Hamster Ovary (CHO) Cells

A culture of 10 ml of non-recombinant CHOExpress® cells (ExcellGene SA) at a density of 1 million cells/ml was co-transfected with a highly efficient transposon-based gene transfer system comprising the plasmid vector pXLG6-coronavirus spike protein comprising the expression cassette for recombinant engineered coronavirus spike protein and the plasmid vector pXLG5 comprising the piggyBac transposase for transposase-mediated gene integration. The non-recombinant CHOExpress® cells acted as a high-performing production host system for large-scale manufacturing using, for example, a bioreactor with an efficient mixing system. The host cells were transformed with the nucleotide sequence encoding the human engineered coronavirus spike protein of interest, where the vector comprising the nucleotide sequence of interest was the pXLG6-coronavirus spike protein vector, and a transformant (i.e. a clonally derived cell population) was isolated expressing the recombinant engineered coronavirus spike protein. The clonally-derived cell lines were selected by single cell cloning and expansion. Among numerous others, stable recombinant clonal CHO cell lines were banked and studied for further analysis.

The cell growth for each of the established cell lines was studied in simple fed-batch (FB or F.B.) processes, using chemically-defined medium (e.g., XLG_E21_7; ExcellGene S.A.) and a single addition of Feed A (ExcellGene S.A.), and demonstrated acceptable cell growth and maintained high cell viability levels for up to 14 days under these conditions. The highest cell densities for certain cell lines, including but not limited to XLG 112 Fed-batch, reached peak growth on or about Day 9 achieving about 18-19×10⁶ cells/ml viable cell density (VCD). By Day 14, the cell density dropped to about 10×10⁶ cells/ml for XLG 112 Fed-batch.

Engineered coronavirus spike protein production in each of the clonal CHO cell lines, i.e., under different culture conditions demonstrated that use of the medium (XLG_E21_7 CDM, ExcellGene S.A.) with an XLG feed (XLG FB) was greatest at Day 14 compared to a commercially available, chemically defined medium (CDM) (e.g., PowerCHO™ 2 Serum-free CDM; LONZA; #BE12-771Q) or an XLG medium alone (e.g., XLG_E21_7 CDM without XLG feed; ExcellGene S.A.) at Day 6. All cell lines were compared in suspension cultures using a process involving a seed density of 0.5×10⁶ cells/ml, a production run time of 14 days, a temperature shift, and under fed-batch conditions with certain feeds (ExcellGene S.A.). The titers of the XLG CHO cell line cultured in an XLG media and feed process (FB) on Day 14 of engineered coronavirus spike protein titer was greater than the same cell line on Day 6 in a commercially available CDM and XLG-medium without XLG feed (i.e., Batch process). Recombinant engineered coronavirus spike protein from different CHO cultures was analyzed by SDS-PAGE for protein expression under different fed-batch processes. The XLG clonal CHO cell line culture was repeatedly found to highly express engineered coronavirus spike protein under various conditions.

Further investigation of the XLG clonal CHO cell line culture as cultured in different fed-batch processes demonstrated that fed batch processes (e.g., Conditions 1 and 3) delivered a titer of about 4 g/L engineered coronavirus spike protein by Day 17. The fed-batch process under Conditions 1 and 3 entailed: a seed density of 0.5×10⁶ cells/ml in a CDM (e.g., XLG_E21_7; ExcellGene S.A.) and grown at a temperature of 37° C. for Day 0-Day 3, temperature of 33° C. for Day 3-Day 17, and supplemented with Feeds 7a and 7b (HyClone™ Cell Boost; GE Healthcare Life Sciences) every other day (EOD) or every day (ED), respectively for Conditions 1 and 3.

TABLE 1 refers to chemically-defined medium feeds, 7a and 7b, as well as to temperature shifts executed during a fed-batch process. The 7a and 7b medium feeds are commercially available (HyClone™ Cell Boost 7a Supplement (SH31026.01); HyClone™ Cell Boost 7b Supplement (SH31027.01); GE Healthcare Life Sciences) and were given to the production process in certain volumes represented in a percent of the total culture volume (EOD: every second day, ED: every day). Temperatures of the production cultures were given over periods of time indicated as Day 0 (d00) to Day 3 (d03); Day 3 to Day 5 (d05) or Day 17 (d17); and Day 5 to Day 17 as indicated. The columns from left to right indicated the number of days, i.e., 7, 11, 14, and 17, respectively, when samples for product concentration in the culture were taken and analyzed for engineered coronavirus spike protein titers.

### Example 5

### Use of Engineered Coronavirus Spike Protein in Immunization and Pseudovirus Protection Experiments in Mice

FIGS. 11A-11F show the results of immunization and resulting neutralizing antibody titers in plasma checked using a panel of spike-pseudotyped viruses. Six mice were vaccinated in each group. FKS05 performed better than or equivalent to other vaccines, against alpha, beta, delta BA1, BA2, BAS, with the exception of BA1 Spike in protecting against BA1 pseudovirus. While there was, as expected, variability in immune responses in the six mice of each group, the overall conclusion was that the FKS antigens are excellent cross reactivity-inducing molecules, solidifying the principle that engineered spikes (or surface proteins from other viruses) make excellent cross-reactive vaccines.

Mice (C57BL/6; n=6) were vaccinated intramuscularly (i.m.) at days 1 and 21 with adjuvant alone (PBS) or with 5 mg each of FKS spike proteins which were formulated in Sepivac SWE™. At 7 days after the first immunization, neutralizing antibody (NAb) titers (EC₅₀) in plasma were determined using a panel of spike-pseudotyped viruses. All FKS antigens induced some level of NAbs against pseudoviruses; however, a subset of antigens (FKS01, FKS03, FKS05, FKS07) were able to induce notable NAbs in mice against all pseudotyped viruses tested (FIGS. 11A-11F). Of note, vaccination with FKS05 spike trimeric antigen resulted in broad cross-reactivity against SARS-CoV-2 variants and was superior to BA.1 spike in neutralizing Alpha, Beta and Delta pseudoviruses (FIGS. 11A-11C). FKS05 also demonstrated equal or greater neutralization of Omicron subvariants (BA.1, BA.2, BA.5) compared to Wuhan spike/SWE (FIGS. 11D-11E).

Vaccination with FKS chimeric antigens induced cross-reactive neutralizing antibodies (NAbs). Mice were vaccinated twice, 3 weeks apart with 5 mg of each spike antigen formulated in SWE adjuvant. One week after the last dose, sera from immunized mice were tested for neutralizing activity against pseudovirus-expressing spike antigen from Alpha virus (A), Beta (B), Delta (C), Omicron BA.1 (D), Omicron BA.2 (E) or Omicron BA.5 (F). The dotted line shows the limit of detection.

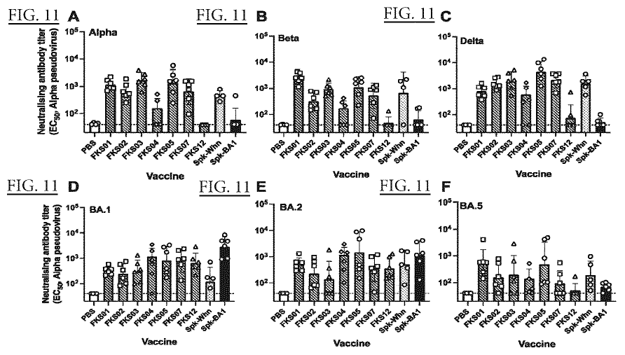

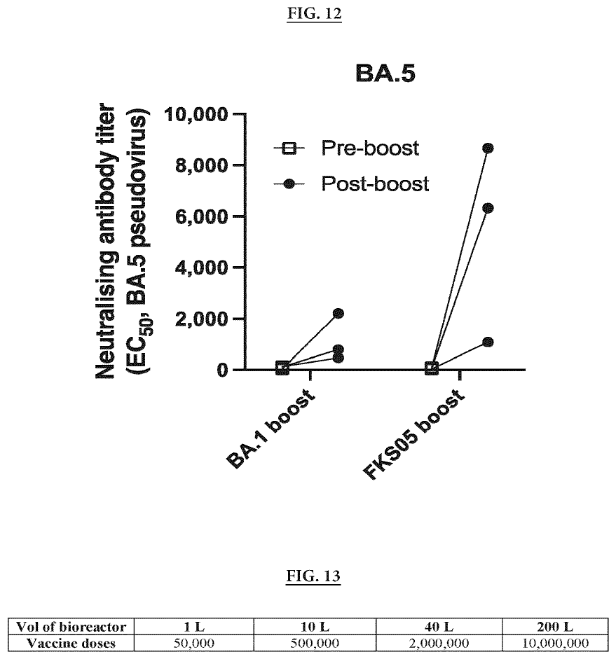

FIG. 12 shows a booster immunization experiment in mice after having been vaccinated twice with the ancestral (Wuhan) trimeric spike formulated in SWE adjuvant. Twenty weeks after the last dose, mice were boosted with a single dose of either BA.1:SWE or FKS05:SWE spike protein and the titer of antibodies able to neutralize the BA.5-spike pseudovirus is shown.

Mice that received 2 doses of Wuhan spike formulated in SWE adjuvant (as a surrogate of standard of care vaccination) were boosted with either Omicron BA.1 spike/SWE or FKS05/SWE. FKS05/SWE was able to boost the levels of NAbs levels against BA.5 spike-expressing pseudovirus; this boosting effect was greater than what had previously been seen when BA.1 spike protein was used as the boosting agent (FIG. 12).

### Example 6

### Production Capacity for Highly Purified Antigen Preparations Based on CHO-Expressed Sike Proteins

Based on highly optimized cell lines and optimized manufacturing approaches as demonstrated in these examples, the yields for a number of doses obtained from different scales of manufacturing runs are shown in FIG. 13. The example was based on the production of a SARS-CoV2 Delta variant derived spike preparation, purified to high purity of >97%, and formulated in a buffer compatible for combination with an adjuvant. Based on prior literature, a purified antigen preparation of 20 μg was sufficient for a single dose in humans. The obtained cell culture yield in bioreactors was 2.5 g/L and the overall recovery yield was 40%. Accordingly, a single 200 liter (L) bioreactor run could deliver 10 million doses. This demonstrated that a global supply for hundreds of millions of doses is entirely feasible using the described method, even when using a relatively small manufacturing facility.

### SPECIFIC EMBODIMENTS

Non-limiting specific embodiments are described below each of which is considered to be within the present disclosure.

Embodiment 1. A protein, comprising:
an engineered coronavirus spike protein comprising an amino acid sequence of at least 90% (e.g., 91%, 92%, 93%, 94%, 95%, 96%, 97%, 98%, 99%, 100%) identity to:

(SEQ ID NO: 2)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QMAYRFNGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG
(FIG. 3E),

wherein the engineered coronavirus spike protein comprises at least one (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) mutation relative to the sequence, wherein the mutation is selected from the group consisting of: T19I; deletion of L24; deletion of P25-P26; deletion of V143; deletion of Y144-Y145; Y145N; deletion of E156; deletion of F157; R158G; deletion of N211; L212I; V213G; insertion of R214; R237M; G252C; D253Y; G257C; G339D; D364Y; V367F; S371L; S373P; S375F; T376A; D405N; R408S; N440K; G446S; I468T; A475V; S477N; E484A; Q493R; G496S; Q498R; Y505H; Y508H; T547K; G566C; R567I; A570D; G593C; G594C; deletion of: Q675, T676, Q677, T678, and N679; N679K; N764K; D796Y; N856K; M902I; Q954H; N969K; L981F; R995M; L996F; G1093C; G1099C; E1188D; and any combinations thereof.

Embodiment 2. A protein, comprising:
an engineered coronavirus spike protein comprising an amino acid sequence of at least 90% (e.g., 91%, 92%, 93%, 94%, 95%, 96%, 97%, 98%, 99%, 100%) identity to:

(SEQ ID NO: 2)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QMAYRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG
(FIG. 3E),

wherein the engineered coronavirus spike protein comprises at least one mutation (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) relative to the sequence, wherein the mutation is selected from the group consisting of: 0-1 mutation of a signal peptide sequence (1-13 aa); 3-16 mutations of an N-terminal domain (13-305 aa); 2-19 mutations of a receptor binding domain (319-541aa); 2-10 mutations of a receptor binding motif (437-508 aa); 0-1 mutation of a fusion peptide sequence (788-806 aa); 0-3 mutations of a heptad repeat 1 (912-984 aa); 0-1 mutation of a heptad repeat 2 (1163-1213 aa); and any combinations thereof.

Embodiment 3. The protein of embodiment 2, wherein the engineered coronavirus spike protein comprises at least one (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) mutation relative to the sequence, wherein the mutation is selected from the group consisting of: T19I; deletion of L24; deletion of P25-P26; deletion of V143; deletion of Y144-Y145; Y145N; deletion of E156; deletion of F157; R158G; deletion of N211; L212I; V213G; insertion of R214; R237M; G252C; D253Y; G257C; G339D; D364Y; V367F; S371L; S373P; S375F; T376A; D405N; R408S; N440K; G446S; I468T; A475V; S477N; E484A; Q493R; G4% S; Q498R; Y505H; Y508H; T547K; G566C; R567I; A570D; G593C; G594C; deletion of: Q675, T676, Q677, T678, and N679; N679K; N764K; D796Y; N856K; M902I; Q954H; N969K; L981F; R995M; L996F; G1093C; G1099C; E1188D; and any combinations thereof.

Embodiment 4. The protein of any one of embodiments 1-3, wherein the engineered coronavirus spike protein comprises an S1 portion and an S2 portion,
wherein the S1 portion comprises a signal peptide domain, an N-terminal domain, a receptor binding domain, and a non-functional furin cleavage site,
wherein the S2 portion comprises a fusion peptide sequence, a heptad repeat 1, and a heptad repeat 2,
wherein the protein comprises a ratio of S1 to S2 selected from the group consisting of: 9/6 or greater, 20/1 or less; and a range of 9/6-20/1 (e.g., 11/6; 17/6; 29/10; 19/6; 20/6; 44/10; 28/6; 15/3; 10/1; 11/1; 20/1).

Embodiment 5. The protein of any one of embodiments 1-4, wherein the engineered coronavirus spike protein comprises at least one (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) mutation relative to the sequence, wherein the mutation is selected from the group consisting of:
deletion of: Y144 and Y145; R158G; A570D; or
T19I; deletion of L24; deletion of: P25 and P26; deletion of V143; deletion of: Y144 and Y145; deletion of N211; L212I; V213G; insertion of R214; T547K; N679K; N764K; D796Y; N856K; Q954H; N969K; L981F; or
T19I; deletion of L24; deletion of: P25 and P26; deletion of V143; deletion of: Y144 and Y145; deletion of N211; L212I; V213G; insertion of R214; T547K; N679K; N764K; D796Y; N856K; Q954H; N969K; L981F; or
deletion of E156; deletion of F157; R158G; G339D; S371L; S373P; S375F; N440K; G446S; S477N; E484A; Q493R; G496S; Q498R; Y505H; or
deletion of E156; deletion of F157; R158G; V367F; I468T; A475V; Y508H; or
deletion of E156; deletion of F157; R158G; G339C; D364Y; Y508H; or
deletion of V143; deletion of: Y144 and Y145; deletion of N211; L212I; insertion of R214; G339D; V367F; I468T; A475V; Y508H; T547K; N679K; N764K; D796Y; N856K; Q954H; N969K; L981F; or
deletion of V143; deletion of: Y144 and Y145; deletion of N211; L212I; insertion of R214; G339C; D364Y; T547K; N679K; N764K; D796Y; N856K; Q954H; N969K; L981F; or
R237M; G252C; D253Y; G257C; G339C; D364Y; G566C; R567I; G593C; G594C; deletion of: Q675, T676, Q677, T678, N679; M902I; R995M; L996F; G1093C; G1099C; E 188D; or
T19I; deletion of L24; deletion of: P25 and P26; deletion of V143; deletion of: Y144 and Y145; deletion of N211; L212I; V213G; insertion of R214; R237M; G252C; D253Y; G257C; G339D; D364Y; S371F; S373P; S375F; T376A; D405N; R408S; N440K; G446S; S477N; E484A; Q493R; G496S; Q498R; Y505H; T547K; G566C; R567I; G593C; G594C; N679K; N764K; D796Y; N856K; Q954H; N969K; L981F; L996F; G1093C; G1099C; E1188D; or
T19I; deletion of L24; deletion of: P25 and P26; deletion of V143; deletion of: Y144 and Y145; deletion of N211; L212I; V213G; insertion of R214; R237M; G252C; D253Y; G257C; E484A; Q493R; G4% S; Q498R; Y505H; T547K; N679K; N764K; D796Y; N856K; Q954H; N969K; L981F; L996F; G1093C; G1099C; E1188D; or
T19I; deletion of P26; Y145N; G339D; S371F; S373P; S375F; N440K; G446S; S477N; E484A; Q493R; G496S; Q498R; Y505H; N679K; N764K; D796Y; Q954H; N969K.

Embodiment 6. A protein, comprising:
an engineered coronavirus spike protein comprising an amino acid sequence of at least 90% (e.g., 91%, 92%, 93%, 94%, 95%, %%, 97%, 98%, 99%, 100%) identity to:

(SEQ ID NO: 2)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QMAYRENGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG
(FIG. 3E),

wherein the engineered coronavirus spike protein comprises a plurality of mutations (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58) relative to the sequence, wherein the mutation is selected from the group consisting of:
S13I; A67V; deletion of H69 and V70; T95I; deletion of Y144 and Y145; W152C; R158G; D253G; L452R; E484K; N501Y; A570D; D614G; Q677H; P681H; F888L; D950N; V1176F; or
T19I; deletion of L24, P25, and P26; A67V; deletion of H69 and V70; T95I; G142D; deletion of V143; deletion of: Y144 and Y145; deletion of N211; L212I; V213G; insertion of R214; L452R; T478K; T547K; D614G; H655Y; N679K; P681H; N764K; D796Y; N856K; Q954H; N969K; L981F; or
T19I; deletion of L24, P25, and P26; A67V; deletion of H69 and V70; T95I; G142D; deletion of V143; deletion of: Y144 and Y145; deletion of N211; L212I; V213G; insertion of R214; K417N; E484K; N501Y; T547K; D614G; H655Y; N679K; P681H; N764K; D796Y; N856K; Q954H; N969K; L981F; or
T19R; deletion of E156; deletion of F157; R158G; G339D; S371L; S373P; S375F; K417N; N440K; G446S; S477N; T478K; E484A; Q493R; G4% S; Q498R; N501Y; Y505H; D614G; P681R; D950N; or
T19R; deletion of E156; deletion of F157; R158G; V367F; L452R; I468T; A475V; T478K; Y508H; D614G; P681R; D950N; or
T19R; deletion of E156; deletion of F157; R158G; G339D; D364Y; L452R; T478K; Y508H; D614G; P681R; D950N; or
A67V; deletion of H69 and V70; T95I; G142D; deletion of V143; deletion of: Y144 and Y145; deletion of N211; L212I; insertion of R214; G339D; V367F; L452R; I468T; A475V; T478K; Y508H; T547K; D614G; H655Y; N679K; P681H; N764K; D796Y; N856K; Q954H; N969K; L981F; or
A67V; deletion of H69 and V70; T95I; G142D; deletion of V143; deletion of: Y144 and Y145; deletion of N211; L212I; insertion of R214; G339C; D364Y; L452R; T478K; T547K; D614G; H655Y; N679K; P681H; N764K; D796Y; N856K; Q954H; N969K; L981F; or
R237M; G252C; D253Y; G257C; G339C; D364Y; G566C; R567I; G593C; G594C; deletion of: Q675, T676, Q677, T678, N679; M902I; R995M; L996F; G1093C; G1099C; E1188D; or
T19I; deletion of L24; deletion of: P25 and P26; A67V; deletion of: H69 and V70; T95I; G142D; deletion of V143; deletion of: Y144 and Y145; deletion of N211; L212I; V213G; insertion of R214; R237M; G252C; D253Y; G257C; G339D; D364Y; S371F; S373P; S375F; T376A; D405N; R408S; K417N; N440K; G446S; S477N; T478K; E484A; Q493R; G496S; Q498R; Y505H; T547K; G566C; R567I; G593C; G594C; D614G; H655Y; N679K; P681H; N764K; D796Y; N856K; Q954H; N969K; L981F; L996F; G1093C; G1099C; E1188D; or
T19I; deletion of L24; deletion of: P25 and P26; A67V; deletion of: H69 and V70; T95I; G142D; deletion of V143; deletion of: Y144 and Y145; deletion of N211; L212I; V213G; insertion of R214; R237M; G252C; D253Y; G257C; L452R; T478K; E484A; Q493R; G496S; Q498R; N501Y; Y505H; T547K; D614G; H655Y; N679K; P681H; N764K; D796Y; N856K; Q954H; N969K; L981F; L996F; G1093C; G1099C; E1188D; or
L18F; T19I; P26S; A67V; deletion of: H69 and V70; T95I; G142D; Y145N; D253G; G339D; S371L; S373P; S375F; K417N; N440K; G446S; S477N; T478K; E484A; Q493R; G496S; Q498R; N501Y; Y505H; D614G; H655Y; N679K; P681H; A701V; N764K; D796Y; D950N; Q954H; N969K.

Embodiment 7. The protein of any one of embodiments 1-6, wherein the engineered coronavirus spike protein comprises a non-functional furin cleavage site.

Embodiment 8. The protein of embodiment 7, wherein the non-functional furin cleavage site comprises GSAS (SEQ ID NO: 28) at positions 682-685 relative to the sequence.

Embodiment 9. The protein of any one of embodiments 1-8, wherein the engineered coronavirus spike protein does not comprise a transmembrane domain or an intracellular tail.

Embodiment 10. The protein of any one of embodiments 1-9, wherein the engineered coronavirus spike protein comprises a T4 foldon motif (e.g., T4 fibritin trimerization foldon motif; GYIPEAPRDGQAYVRKDGEWVLLSTFL (SEQ ID NO: 3) at amino acid positions 1211-1237 relative to the modified amino acid sequence of FIG. 3E).

Embodiment 11. The protein of any one of embodiments 1-10, wherein the protein comprises more than one engineered coronavirus spike protein, wherein each engineered coronavirus spike protein comprises a virus variant-spike monomer capable of forming a homo-trimeric structure or a hetero-trimeric structure.

Embodiment 12. A protein, comprising:
an engineered coronavirus spike protein comprising an amino acid sequence of at least 90% identity to:

(SEQ ID NO: 2)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QMAYRFNGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG
(FIG. 3E),

wherein the protein comprises more than one engineered coronavirus spike protein,
wherein each engineered coronavirus spike protein comprises a virus variant-spike monomer capable of forming a homo-trimeric structure or a hetero-trimeric structure.

Embodiment 13. A nucleic acid molecule comprising a nucleotide sequence encoding an amino acid sequence of a protein of any one of embodiments 1-12.

Embodiment 14. The nucleic acid molecule of embodiment 11, wherein the nucleic acid molecule comprises a vector.

Embodiment 15. The nucleic acid molecule of embodiment 14, wherein the vector is an expression vector or a viral vector.

Embodiment 16. The nucleic acid molecule of embodiment 13, wherein the nucleic acid molecule comprises a mRNA.

Embodiment 17. A pharmaceutical composition comprising: a protein of any one of embodiments 1-12 or a nucleic acid molecule of any one of embodiments 13-16, wherein the pharmaceutical composition comprises a pharmaceutically acceptable vehicle.

Embodiment 18. The pharmaceutical composition of embodiment 17, further comprises an adjuvant.

Embodiment 19. A host cell comprising the nucleic acid molecule of any one of embodiments 13-16.

Embodiment 20. A method, comprising:
prophylactically treating a coronavirus infection or disease associated with a coronavirus infection in a subject, wherein the treating comprises,
administering to the subject an effective amount of the protein of any one of embodiments 1-12, the nucleic acid molecule of any one of embodiments 13-16, or the pharmaceutical composition of any one of embodiments 17-18.

Embodiment 21. The method of embodiment 20, wherein the coronavirus infection comprises COVID-19.

Embodiment 22. The method of any one of embodiments 20-21, wherein the coronavirus infection comprises a COVID-19 variant selected from the group consisting of: Wuhan; alpha; beta; gamma; delta; epsilon; eta; iota; lambda; mu; omicron BA.1; omicron BA.2; and zeta.

Embodiment 23. Use of the protein of any one of embodiments 1-12, the nucleic acid molecule of any one of embodiments 13-16 or the pharmaceutical composition of any one of embodiments 17-18 for prophylactically treating a coronavirus infection or disease associated with a coronavirus infection.

Embodiment 24. The use of embodiment 23, wherein the coronavirus infection comprises COVID-19.

Embodiment 25. The use of any one of embodiments 23-24, wherein the coronavirus infection comprises a COVID-19 variant selected from the group consisting of: Wuhan; alpha; beta; gamma; delta; epsilon; eta; iota; lambda; mu; omicron BA.1; omicron BA.2; and zeta.

Embodiment 26. A method for producing a recombinant engineered coronavirus spike protein, comprising:
a. introducing into a host cell, an expression vector comprising a nucleic acid molecule encoding the engineered coronavirus spike protein of FIG. 2A-FIG. 2F and FIG. 3A-FIG. 3R;
b. culturing the host cell under conditions which allow for expression of the recombinant engineered coronavirus spike protein; and
c. isolating the recombinant engineered coronavirus spike protein from the cultured host cell, thereby producing the recombinant engineered coronavirus spike protein.

Embodiment 27. The method according to embodiment 26, wherein the nucleic acid molecule comprises a nucleic acid sequence encoding an engineered coronavirus spike CHO-cell codon-optimized sequence (and is driven by an optimized constitutive promoter).

Embodiment 28. The method according to embodiment 26 or embodiment 27, where the introducing step comprises co-transfecting the recombinant engineered coronavirus spike expression vector and an expression vector encoding a transposase.

Embodiment 29. The method according to embodiment 28, wherein the transposase is a piggyBac transposase.

Embodiment 30. The method according to any of embodiments 26-29, wherein the host cell is a eukaryotic cell.

Embodiment 31. The method according to any of embodiments 26-30, wherein the host cell is a Chinese hamster ovary (CHO) cell line.

Embodiment 32. The method according to embodiment 31, wherein the CHO cell line is a modified CHO cell line.

Embodiment 33. The method according to any of embodiments 26-32, wherein the culturing step occurs in a culture medium, and the culture medium contains less than about 5% (vol/vol) of non-human-derived components.

Embodiment 34. The method according to any of embodiments 26-33, wherein the culturing step occurs in a culture medium, and the culture medium contains less than about 2% (vol/vol) of non-human-derived components.

Embodiment 35. The method according to any of embodiments 26-34, wherein the produced recombinant engineered coronavirus spike protein comprises an amount of about 1 g/L to about 10 g/L of recombinant engineered coronavirus spike protein.

Embodiment 36. The method according to any of embodiments 26-35, wherein the produced recombinant engineered coronavirus spike protein comprises an amount of about 2 g/L to about 6 g/L of recombinant engineered coronavirus spike protein.

Embodiment 37. The method according to any of embodiments 26-36, wherein the culturing step comprises: selecting the host cell with the nucleic acid molecule expressing the recombinant engineered coronavirus spike protein, wherein the selected cells are clonally-derived cells expressing recombinant engineered coronavirus spike.

Embodiment 38. The method according to embodiment 37, wherein the selecting step comprises:
a. culturing the clonally-derived cells expressing recombinant engineered coronavirus spike in a culture medium;
b. feeding the clonally-derived cells expressing recombinant engineered coronavirus spike with at least one feed;
c. maintaining the culture medium at a cell culture temperature;
d. decreasing the cell culture temperature; and
e. culturing the clonally-derived cells at the decreased cell culture temperature, wherein the clonally-derived cells express the recombinant engineered coronavirus spike protein at a titer of about 1 g/L or greater.

Embodiment 39. The method according to any of embodiments 37-38, wherein the clonally-derived cells express recombinant engineered coronavirus spike protein at a titer of greater than about 2 g/L.

Embodiment 40. The method according to any of embodiments 37-39, wherein the clonally-derived cells express recombinant engineered coronavirus spike protein at a titer of greater than about 4 g/L.

Embodiment 41. The method according to any of embodiments 38-40, wherein the cell culture temperature ranges from about 35° C. to about 38° C.

Embodiment 42. The method according to any of embodiments 38-40, wherein the cell culture temperature is maintained from Day 0 to Day 3 or Day 0 to Day 5.

Embodiment 43. The method according to any of embodiments 38-42, wherein the decreased cell culture temperature ranges from about 25° C. to about 34° C.

Embodiment 44. The method according to any of embodiments 38-43, wherein the cell culture medium is at a decreased cell culture temperature from Day 3 to Day 17, Day 3 to Day 5, Day 5 to Day 17, or combinations thereof.

Embodiment 45. The method according to any of embodiments 38-44, wherein the at least one feed comprises a neutral feed.

Embodiment 46. The method according to embodiment 45, wherein the neutral feed is in a volume ranging from about 1% to about 8% of the total cell culture volume.

Embodiment 47. The method according to any one of embodiments 38-46, wherein the at least one feed comprises an alkaline feed.

Embodiment 48. The method according to embodiment 47, wherein the alkaline feed is in a volume ranging from about 0.1% to about 0.8% of the total cell culture volume.

Embodiment 49. The method according to any of embodiments 38-48, wherein the at least one feed comprises a neutral feed and an alkaline feed.

Embodiment 50. The method according to embodiment 49, wherein the alkaline feed is in an amount one-tenth (1/10) of an amount of a neutral feed.

Embodiment 51. The method according to any of embodiments 38-50, wherein the feeding step occurs every day.

Embodiment 52. The method according to any of embodiments 38-50, wherein the feeding step occurs every other day.

Embodiment 53. The method according to any of embodiments 38-52, wherein the culturing step comprises an osmolarity of the cell culture of about 550 mOsm/kg or greater.

Embodiment 54. The method according to any of embodiments 38-53, wherein the culturing step comprises an osmolarity of the cell culture of about 550 mOsm/kg or greater at Day 5 or later.

Embodiment 55. A method for producing a recombinant engineered coronavirus spike protein, comprising:
a. introducing into a eukaryotic host cell, a first nucleic acid sequence encoding a engineered coronavirus spike protein and at least an additional nucleic acid sequence encoding a transposase;
b. culturing the eukaryotic host cell under conditions which allow expression of the first nucleic acid sequence encoding a engineered coronavirus spike protein;
c. selecting the eukaryotic host cell with the nucleic acid molecule expressing a engineered coronavirus spike protein, wherein the selected cells are clonally-derived cells expressing recombinant engineered coronavirus spike protein; and
d. isolating the recombinant engineered coronavirus spike protein from the clonally-derived cells, thereby producing the recombinant engineered coronavirus spike protein.

Embodiment 56. The method according to embodiment 55, wherein the eukaryotic host cell is transformed with the nucleic acid sequence encoding a recombinant engineered coronavirus spike protein.

Embodiment 57. The method according to embodiment 55 or embodiment 54, wherein the step of isolating comprises purifying the recombinant engineered coronavirus spike protein.

Embodiment 58. The method according to embodiment 57, wherein the step of purifying is by at least one of: size exclusion chromatography, affinity chromatography, ion exchange chromatography, hydrophobic interaction chromatography, reverse phase chromatography, gel filtration, magnetic bead separation, selective precipitation, molecular weight-based membrane filtration or exclusion, buffer exchange, virus filtration, pH-based inactivation of viruses, and the like.

Embodiment 59. The method according to any of embodiments 26-58, wherein the isolated recombinant engineered coronavirus spike protein has a purity of about or greater than about 95%.

Embodiment 60. The method according to any of embodiments 26-59, wherein the isolated recombinant engineered coronavirus spike protein has a purity of about or greater than about 98%.

Embodiment 61. An expression vector, comprising: a nucleic acid molecule containing a nucleotide sequence encoding a recombinant engineered coronavirus spike protein, wherein the nucleic acid molecule is positioned in a multiple cloning site; an intron upstream of the nucleic acid molecule; a cytomegalovirus (CMV) promoter upstream of an intron; a 5′ Inverted Terminal Repeat (5′ ITR) upstream of the CMV promoter, a poly-adenosine tail signal sequence downstream of the nucleic acid molecule; a replication origin sequence downstream of the nucleic acid molecule; a selectable marker sequence downstream of the replication origin sequence; and a 3′ Inverted Terminal Repeat (3′ ITR) downstream of the selectable marker sequence.

Embodiment 62. The expression vector according to embodiment 61, wherein the selectable marker sequence is a puromycin resistance gene.

Embodiment 63. The expression vector according to any of embodiments 61-62, wherein the nucleic acid molecule and the selectable marker sequence are positioned in opposite reading frames and in between the 5′ ITR and the 3′ ITR.

Embodiment 64. The expression vector according to any of embodiments 61-63, wherein the nucleotide sequence encodes a recombinant engineered coronavirus spike polypeptide sequence of at least one of: FIG. 2A-FIG. 2F and FIG. 3A-FIG. 3R.

Embodiment 65. The expression vector according to any of embodiments 61-64, wherein the nucleotide sequence encoding a recombinant engineered coronavirus spike polypeptide sequence comprises a sequence of at least one of: FIG. 2A-FIG. 2F and FIG. 3A-FIG. 3R.

Embodiment 66. The expression vector according to any of embodiments 61-65, wherein the expression vector comprises FIG. 3A-FIG. 3R.

Embodiment 67. A recombinant engineered coronavirus spike protein, comprising a polypeptide sequence having about 95% identity to FIG. 3E.

Embodiment 68. The recombinant engineered coronavirus spike protein according to embodiment 67, comprising a polypeptide sequence of FIG. 3E having a mutation selected from the group consisting of: deletion of Y144; deletion of Y145; R158G; A570D; and any combinations thereof.

Embodiment 69. A composition, comprising a recombinant engineered coronavirus spike protein produced by the method of any one of embodiments 26-60, and a pharmaceutically acceptable vehicle.

Embodiment 70. A method for producing a recombinant engineered coronavirus spike protein, comprising: culturing a host cell with a first nucleic acid sequence encoding an engineered coronavirus spike protein of FIG. 2A-FIG. 2F and FIG. 3A-FIG. 3R and at least a second nucleic acid sequence encoding a transposase during generation of the cells comprising recombinant engineered coronavirus spike protein, wherein the culturing step occurs at a first period of time at a first temperature and at a second period of time at a second temperature, and optionally at a third period of time at a third temperature.

Embodiment 71. The method of embodiment 70, wherein the second temperature is less than the first temperature.

Embodiment 72. The method of embodiment 71, wherein the third temperature is less than the second temperature.

Embodiment 73. The method of embodiment 72, wherein the first temperature ranges from about 31° C. to about 37° C.

Embodiment 74. The method of embodiment 73, wherein the second temperature ranges from about 31° C. to about 37° C.

Embodiment 75. The method of embodiment 74, wherein the third temperature ranges from about 31° C. to about 37° C.

Embodiment 76. The method of embodiment 75, wherein the first period of time ranges from about 1-20 days.

Embodiment 77. The method of embodiment 76, wherein the second period of time ranges from about 1-20 days.

Embodiment 78. The method of embodiment 77, wherein the third period of time ranges from about 1-20 days.

Embodiment 79. The method of embodiment 78, wherein the culturing step further comprises adding a first feed and a second feed.

Embodiment 80. The method of embodiment 79, wherein the adding step occurs every other day.

Embodiment 81. The method of embodiment 80, wherein the adding step occurs every day.

Embodiment 82. The method of the embodiment 81, wherein the culture for production is oxygenated with air only under avoidance of pure oxygen.

Embodiment 83 The method of embodiments 26-60; the expression vector of embodiments 61-66; the recombinant engineered coronavirus spike protein of embodiments 67-68; or the composition of embodiment 69, wherein the recombinant engineered spike protein (e.g., FrankenSpikes, MonsterSpikes, or Monster-FrankenSpikes) is used as a prophylactic treatment against Coronavirus-induced disease or infection.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and can be employed in the practice of the disclosure.

1. A protein, comprising:
an engineered coronavirus spike protein comprising an amino acid sequence of at least 90% identity to:
(SEQ ID NO: 2)
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPGSASSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSPIEDLLFNKVTLADAGFIKQYGDCLGDIAARDLICAQKF
NGLTVLPPLLTDEMIAQYTSALLAGTITSGWTFGAGPALQIPFPM
QMAYRFNGIGVTQNVLYENQKLIANQFNSAIGKIQDSLSSTPSAL
GKLQDVVNQNAQALNTLVKQLSSNFGAISSVLNDILSRLDPPEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQGSGYIPE
APRDGQAYVRKDGEWVLLSTFLGRSLEVLFQGPG
(FIG. 3E),
wherein the engineered coronavirus spike protein comprises at least one mutation relative to the sequence, wherein the at least one mutation is selected from the group consisting of: T19I; deletion of L24; deletion of P25-P26; deletion of V143; deletion of Y144-Y145; Y145N; deletion of E156; deletion of F157; R158G; deletion of N211; L212I; V213G; insertion of R214; R237M; G252C; D253Y; G257C; G339D; D364Y; V367F; S371L; S373P; S375F; T376A; D405N; R408S; N440K; G446S; I468T; A475V; S477N; E484A; Q493R; G496S; Q498R; Y505H; Y508H; T547K; G566C; R567I; A570D; G593C; G594C; deletion of: Q675, T676, Q677, T678, and N679; N679K; N764K; D796Y; N856K; M902I; Q954H; N969K; L981F; R995M; L996F; G1093C; G1099C; E1188D; and any combinations thereof; or
wherein the engineered coronavirus spike protein comprises at least one mutation relative to the sequence, wherein the mutation is selected from the group consisting of: 0-1 mutation of a signal peptide sequence (1-13 aa); 3-16 mutations of an N-terminal domain (13-305 aa); 2-19 mutations of a receptor binding domain (319-541aa); 2-10 mutations of a receptor binding motif (437-508 aa); 0-1 mutation of a fusion peptide sequence (788-806 aa); 0-3 mutations of a heptad repeat 1 (912-984 aa); 0-1 mutation of a heptad repeat 2 (1163-1213 aa); and any combinations thereof; or
wherein the engineered coronavirus spike protein comprises a plurality of mutations relative to the sequence, wherein the plurality of mutations is selected from the group consisting of:
S13I; A67V; deletion of H69 and V70; T95I; deletion of Y144 and Y145; W152C; R158G; D253G; L452R; E484K; N501Y; A570D; D614G; Q677H; P681H; F888L; D950N; V1176F; or
T19I; deletion of L24, P25, and P26; A67V; deletion of H69 and V70; T95I; G142D; deletion of V143; deletion of: Y144 and Y145; deletion of N211; L212I; V213G; insertion of R214; L452R; T478K; T547K; D614G; H655Y; N679K; P681H; N764K; D796Y; N856K; Q954H; N969K; L981F; or
T19I; deletion of L24, P25, and P26; A67V; deletion of H69 and V70; T95I; G142D; deletion of V143; deletion of: Y144 and Y145; deletion of N211; L212I; V213G; insertion of R214; K417N; E484K; N501Y; T547K; D614G; H655Y; N679K; P681H; N764K; D796Y; N856K; Q954H; N969K; L981F; or
T19R; deletion of E156; deletion of F157; R158G; G339D; S371L; S373P; S375F; K417N; N440K; G446S; S477N; T478K; E484A; Q493R; G496S; Q498R; N501Y; Y505H; D614G; P681R; D950N; or
T19R; deletion of E156; deletion of F157; R158G; V367F; L452R; I468T; A475V; T478K; Y508H; D614G; P681R; D950N; or
T19R; deletion of E156; deletion of F157; R158G; G339D; D364Y; L452R; T478K; Y508H; D614G; P681R; D950N; or
A67V; deletion of H69 and V70; T95I; G142D; deletion of V143; deletion of: Y144 and Y145; deletion of N211; L212I; insertion of R214; G339D; V367F; L452R; I468T; A475V; T478K; Y508H; T547K; D614G; H655Y; N679K; P681H; N764K; D796Y; N856K; Q954H; N969K; L981F; or
A67V; deletion of H69 and V70; T95I; G142D; deletion of V143; deletion of: Y144 and Y145; deletion of N211; L212I; insertion of R214; G339C; D364Y; L452R; T478K; T547K; D614G; H655Y; N679K; P681H; N764K; D796Y; N856K; Q954H; N969K; L981F; or
R237M; G252C; D253Y; G257C; G339C; D364Y; G566C; R567I; G593C; G594C; deletion of: Q675, T676, Q677, T678, N679; M902I; R995M; L996F; G1093C; G1099C; E1188D; or
T19I; deletion of L24; deletion of: P25 and P26; A67V; deletion of: H69 and V70; T95I; G142D; deletion of V143; deletion of: Y144 and Y145; deletion of N21; L212I; V213G; insertion of R214; R237M; G252C; D253Y; G257C; G339D; D364Y; S371F; S373P; S375F; T376A; D405N; R408S; K417N; N440K; G446S; S477N; T478K; E484A; Q493R; G496S; Q498R; Y505H; T547K; G566C; R567I; G593C; G594C; D614G; H655Y; N679K; P681H; N764K; D796Y; N856K; Q954H; N969K; L981F; L996F; G1093C; G1099C; E1188D; or
T19I; deletion of L24; deletion of: P25 and P26; A67V; deletion of: H69 and V70; T95I; G142D; deletion of V143; deletion of: Y144 and Y145; deletion of N211; L212I; V213G; insertion of R214; R237M; G252C; D253Y; G257C; L452R; T478K; E484A; Q493R; G496S; Q498R; N501Y; Y505H; T547K; D614G; H655Y; N679K; P681H; N764K; D796Y; N856K; Q954H; N969K; L981F; L996F; G1093C; G1099C; E1188D; or
L18F; T19I; P26S; A67V; deletion of: H69 and V70; T95I; G142D; Y145N; D253G; G339D; S371L; S373P; S375F; K417N; N440K; G446S; S477N; T478K; E484A; Q493R; G496S; Q498R; N501Y; Y505H; D614G; H655Y; N679K; P681H; A701V; N764K; D796Y; D950N; Q954H; N969K; or
wherein the protein comprises more than one engineered coronavirus spike protein, wherein each engineered coronavirus spike protein comprises a virus variant-spike monomer capable of forming a homo-trimeric structure or a hetero-trimeric structure.

2. (canceled)

3. (canceled)

4. The protein of claim 1, wherein the engineered coronavirus spike protein comprises an S1 portion and an S2 portion,
wherein the S1 portion comprises a signal peptide domain, an N-terminal domain, a receptor binding domain, and a non-functional furin cleavage site,
wherein the S2 portion comprises a fusion peptide sequence, a heptad repeat 1, and a heptad repeat 2,
wherein the protein comprises a ratio of S1 to S2 selected from the group consisting of: 9/6 or greater; 20/1 or less; and a range of 9/6-20/1.

5. The protein of claim 1, wherein the engineered coronavirus spike protein comprises at least one mutation relative to the sequence, wherein the at least one mutation is selected from the group consisting of:
deletion of: Y144 and Y145; R158G; A570D; or
T19I; deletion of L24; deletion of: P25 and P26; deletion of V143; deletion of: Y144 and Y145; deletion of N211; L212I; V213G; insertion of R214; T547K; N679K; N764K; D796Y; N856K; Q954H; N969K; L981F; or
deletion of E156; deletion of F157; R158G; G339D; S371L; S373P; S375F; N440K; G446S; S477N; E484A; Q493R; G496S; Q498R; Y505H; or
deletion of E156; deletion of F157; R158G; V367F; I468T; A475V; Y508H; or
deletion of E156; deletion of F157; R158G; G339C; D364Y; Y508H; or
deletion of V143; deletion of: Y144 and Y145; deletion of N211; L212I; insertion of R214; G339D; V367F; I468T; A475V; Y508H; T547K; N679K; N764K; D796Y; N856K; Q954H; N969K; L981F; or
deletion of V143; deletion of: Y144 and Y145; deletion of N211; L212I; insertion of R214; G339C; D364Y; T547K; N679K; N764K; D796Y; N856K; Q954H; N969K; L981F; or
R237M; G252C; D253Y; G257C; G339C; D364Y; G566C; R567I; G593C; G594C; deletion of: Q675, T676, Q677, T678, N679; M902I; R995M; L996F; G1093C; G1099C; E1188D; or
T19I; deletion of L24; deletion of: P25 and P26; deletion of V143; deletion of: Y144 and Y145; deletion of N211; L212I; V213G; insertion of R214; R237M; G252C; D253Y; G257C; G339D; D364Y; S371F; S373P; S375F; T376A; D405N; R408S; N440K; G446S; S477N; E484A; Q493R; G496S; Q498R; Y505H; T547K; G566C; R567I; G593C; G594C; N679K; N764K; D796Y; N856K; Q954H; N969K; L981F; L996F; G1093C; G1099C; E1188D; or
T19I; deletion of L24; deletion of: P25 and P26; deletion of V143; deletion of: Y144 and Y145; deletion of N211; L212I; V213G; insertion of R214; R237M; G252C; D253Y; G257C; E484A; Q493R; G496S; Q498R; Y505H; T547K; N679K; N764K; D796Y; N856K; Q954H; N969K; L981F; L996F; G1093C; G1099C; E1188D; or
T19I; deletion of P26; Y145N; G339D; S371F; S373P; S375F; N440K; G446S; S477N;
E484A; Q493R; G496S; Q498R; Y505H; N679K; N764K; D796Y; Q954H; N969K.

6. (canceled)

7. The protein of claim 1, wherein the engineered coronavirus spike protein comprises a non-functional furin cleavage site.

8. The protein of claim 7, wherein the non-functional furin cleavage site comprises GSAS (SEO ID NO; 28) at positions 682-685 relative to the sequence.

9. The protein of claim 1, wherein the engineered coronavirus spike protein does not comprise a transmembrane domain or an intracellular tail.

10. The protein of claim 1, wherein the engineered coronavirus spike protein comprises a T4 foldon motif.

11. The protein of claim 1, wherein the protein comprises more than one engineered coronavirus spike protein, wherein each engineered coronavirus spike protein comprises a virus variant-spike monomer capable of forming a homo-trimeric structure or a hetero-trimeric structure.

12. The protein of claim 1, wherein a trimeric protein comprises more than one engineered coronavirus monomer of the spike protein, wherein a preparation of engineered coronavirus spike proteins comprises a virus variant spike monomer capable of forming a hetero-trimeric structure with a specific molecule derived from a specific SARS-CoV2 virus variant or a specific FrankenSpike.

13. (canceled)

14. A nucleic acid molecule comprising a nucleotide sequence encoding an amino acid sequence of claim 1.

15. The nucleic acid molecule of claim 14, wherein the nucleic acid molecule comprises a vector or mRNA.

16. The nucleic acid molecule of claim 15, wherein the vector is an expression vector or a viral vector.

17. (canceled)

18. A pharmaceutical composition comprising: a protein of claim 1 or a nucleic acid molecule of 14, wherein the pharmaceutical composition comprises a pharmaceutically acceptable vehicle.

19. The pharmaceutical composition of claim 18, further comprises an adjuvant.

20. A host cell comprising the nucleic acid molecule of claim 14.

21. A method, comprising:
prophylactically treating a coronavirus infection or disease associated with a coronavirus infection in a subject, wherein the treating comprises,
administering to the subject an effective amount of the protein of claim 1, the nucleic acid molecule of claim 14, or the pharmaceutical composition of claim 18.

22. The method of claim 21, wherein the coronavirus infection comprises COVID-19.

23. The method of claim 21, wherein the coronavirus infection comprises a COVID-19 variant selected from the group consisting of: Wuhan; alpha; beta; gamma; delta; epsilon; eta; iota; lambda; mu; omicron BA.1; omicron BA.2; omicron BA.5; omicron BQ.1.1.; omicron XBB.1.5; and zeta.

24. The method of claim 21, wherein the subject has not been vaccinated against coronavirus infection and has not been infected by SARS-CoV2.

25. The method of claim 21, wherein the protein of claim 1, the nucleic acid molecule of claim 14, or the pharmaceutical composition of claim 18 is administered as a booster vaccination of the subject who has previously been vaccinated against coronavirus infection or has previously been infected by the SARS-CoV-2 virus.
